SEQUENCE LISTING (110 sequences)

SEQ ID NO: 1 - DNA, 168 bp, Synthetic construct
  source 1..168: mol_type = other DNA
tgtctcactaggtcctctcagctcttcccaccaccattgccttcttcttccttctgagtctaaccaccaccattgccttatctaccattctgagtcttaccaccaccattgccatttcattatctacgagtctatccaccaccattgcaggagtttcagtttcgacgc

SEQ ID NO: 2 - DNA, 168 bp, Synthetic construct
  source 1..168: mol_type = other DNA
gcgtcgaaactgaaactcctgcaatggtggtggatagactcgtagataatgaaatggcaatggtggtggtaagactcagaatggtagataaggcaatggtggtggttagactcagaaggaagaagaaggcaatggtggtgggaagagctgagaggacctagtgagaca

SEQ ID NO: 3 - DNA, 168 bp, Synthetic construct
  source 1..168: mol_type = other DNA
tgtctcactaggtcctctcattccgagggagatctacactgctcgtcttaatatacggaataaccagcccgcggctccaaaccgactagattctttctcgagttatccacatacatcggtcttatattgttggtgatacccaaacaataggagtttcagtttcgacgc

SEQ ID NO: 4 - DNA, 168 bp, Synthetic construct
  source 1..168: mol_type = other DNA
gcgtcgaaactgaaactcctattgtttgggtatcaccaacaatataagaccgatgtatgtggataactcgagaaagaatctagtcggtttggagccgcgggctggttattccgtatattaagacgagcagtgtagatctccctcggaatgagaggacctagtgagaca

SEQ ID NO: 5 - DNA, 179 bp, Synthetic construct
  source 1..179: mol_type = other DNA
tgtctcactaggtcctctcagctcttcccaccaccattgccttcttcttccttctgagtctaaccaccaccattgccttatctaccattctgagtcttaccaccaccattgccatttcattatctacgagtctatccaccaccattgcaggagtttcagtttcgacgcaaaaaaaaaaa

SEQ ID NO: 6 - DNA, 179 bp, Synthetic construct
  source 1..179: mol_type = other DNA
tttttttttttgcgtcgaaactgaaactcctgcaatggtggtggatagactcgtagataatgaaatggcaatggtggtggtaagactcagaatggtagataaggcaatggtggtggttagactcagaaggaagaagaaggcaatggtggtgggaagagctgagaggacctagtgagaca

SEQ ID NO: 7 - DNA, 179 bp, Synthetic construct
  source 1..179: mol_type = other DNA
tgtctcactaggtcctctcattccgagggagatctacactgctcgtcttaatatacggaataaccagcccgcggctccaaaccgactagattctttctcgagttatccacatacatcggtcttatattgttggtgatacccaaacaataggagtttcagtttcgacgcaaaaaaaaaaa

SEQ ID NO: 8 - DNA, 179 bp, Synthetic construct
  source 1..179: mol_type = other DNA
tttttttttttgcgtcgaaactgaaactcctattgtttgggtatcaccaacaatataagaccgatgtatgtggataactcgagaaagaatctagtcggtttggagccgcgggctggttattccgtatattaagacgagcagtgtagatctccctcggaatgagaggacctagtgagaca

SEQ ID NO: 9 - DNA, 20 bp, Synthetic construct
  source 1..20: mol_type = other DNA
tgtctcactaggtcctctca

SEQ ID NO: 10 - DNA, 20 bp, Synthetic construct
  source 1..20: mol_type = other DNA
gcgtcgaaactgaaactcct

SEQ ID NO: 11 - DNA, 20 bp, Synthetic construct
  source 1..20: mol_type = other DNA
ccaccattgccttcttcttc

SEQ ID NO: 12 - DNA, 20 bp, Synthetic construct
  source 1..20: mol_type = other DNA
ggatagactcgtagataatg

SEQ ID NO: 13 - DNA, 20 bp, Synthetic construct
  source 1..20: mol_type = other DNA
ccgagggagatctacactgc

SEQ ID NO: 14 - DNA, 21 bp, Synthetic construct
  source 1..21: mol_type = other DNA
gtatcaccaacaatataagac

SEQ ID NO: 15 - DNA, 43 bp, Synthetic construct
  source 1..43: mol_type = other DNA
cactaggtcctctcagctcttcccaccaccattgccttcttct

SEQ ID NO: 16 - DNA, 15 bp, Synthetic construct
  source 1..15: mol_type = other DNA
agggaagagctgaga

SEQ ID NO: 17 - DNA, 23 bp, Synthetic construct
  source 1..23: mol_type = other DNA
agggaagagctgagaggacctag

SEQ ID NO: 18 - DNA, 44 bp, Synthetic construct
  source 1..44: mol_type = other DNA
aagaagaaggcaatggtggtgggaagagctgagaggacctagtg

SEQ ID NO: 19 - DNA, 20 bp, Synthetic construct
  source 1..20: mol_type = other DNA
tgagtaccgcttcacacaga

SEQ ID NO: 20 - DNA, 20 bp, Synthetic construct
  source 1..20: mol_type = other DNA
cggaggcattgagtacgatg

SEQ ID NO: 21 - DNA, 30 bp, Synthetic construct
  source 1..30: mol_type = other DNA
gctcttcccaccaccattgccttcttcttg

SEQ ID NO: 22 - DNA, 30 bp, Synthetic construct
  source 1..30: mol_type = other DNA
caagaagaaggcaatggtggtgggaagagc

SEQ ID NO: 23 - DNA, 133 bp, Synthetic construct
  source 1..133: mol_type = other DNA
tgtctcactaggtcctctcagctcttcccaccaccattgccttcttctttactgctagaacgtgcgtctagcatacacagatcacgcgtgcaagactcatcgtaatctacctgaggagtttcagtttcgacgc

SEQ ID NO: 24 - DNA, 133 bp, Synthetic construct
  source 1..133: mol_type = other DNA
tgtctcactaggtcctctcagctcttcccaccaccattgccttcttctttagtctgtgagtcatgagatcactcgagtgcactgtgcgttgctaccattcacagacgtagctgaggagtttcagtttcgacgc

SEQ ID NO: 25 - DNA, 133 bp, Synthetic construct
  source 1..133: mol_type = other DNA
tgtctcactaggtcctctcagctcttcccaccaccattgccttcttctttctcgactgcgtcgcagattcgacggagtctcagcgtagtgctctactcagagtctcagtatcgaggagtttcagtttcgacgc

SEQ ID NO: 26 - DNA, 133 bp, Synthetic construct
  source 1..133: mol_type = other DNA
tgtctcactaggtcctctcagctcttcccaccaccattgccttcttcttttgccagatcgtcgcatcatcacatacgcagtagatccgtagatgatagtctacgtcgtcgcataggagtttcagtttcgacgc

SEQ ID NO: 27 - DNA, 133 bp, Synthetic construct
  source 1..133: mol_type = other DNA
tgtctcactaggtcctctcagctcttcccaccaccattgccttcttctttgagtgcactcacgcagtctagagcgcttctagcacacgatgctagcatcagtgaacaagactaaggagtttcagtttcgacgc

SEQ ID NO: 28 - DNA, 133 bp, Synthetic construct
  source 1..133: mol_type = other DNA
tgtctcactaggtcctctcagctcttcccaccaccattgccttcttctttgcatctcagcgtagtgctctactcagacagatcgtcatgagatgaacggactgcgcttcgagcaggagtttcagtttcgacgc

SEQ ID NO: 29 - DNA, 133 bp, Synthetic construct
  source 1..133: mol_type = other DNA
tgtctcactaggtcctctcagctctcctacagagccgagcacactcgagaatgctgctgacacgttgctgatagtcttgcactcacgcagtctagagcgctatccacctgagtaggagtttcagtttcgacgc

SEQ ID NO: 30 - DNA, 133 bp, Synthetic construct
  source 1..133: mol_type = other DNA
tgtctcactaggtcctctcaagtctcacactgtgttcacgatgatagactgcttctactacacgatcttgataggctctggagcgtagtgcttgtctcagatcttcgagccgaaggagtttcagtttcgacgc

SEQ ID NO: 31 - DNA, 133 bp, Synthetic construct
  source 1..133: mol_type = other DNA
tgtctcactaggtcctctcaagtcatgagtgcacttagcgtagttgtcatacttctagcacaacagcttcactcgacctggtgacacgtgcacatgtggctgtgcacctgtctaggagtttcagtttcgacgc

SEQ ID NO: 32 - DNA, 133 bp, Synthetic construct
  source 1..133: mol_type = other DNA
tgtctcactaggtcctctcaagtacaatcctgactacaacagcttcgtcgtctcagctatagacagcttgttcgagactgtgagtcatggcatagcatgagatctcgcagctaaggagtttcagtttcgacgc

SEQ ID NO: 33 - DNA, 133 bp, Synthetic construct
  source 1..133: mol_type = other DNA
tgtctcactaggtcctctcaagttaggcttctgtgatcgcaacatcgacgatgtctcaggtcgcaagatcacatgcacagtagacacgttgctagcattcttcatgtgacagcaggagtttcagtttcgacgc

SEQ ID NO: 34 - DNA, 133 bp, Synthetic construct
  source 1..133: mol_type = other DNA
tgtctcactaggtcctctcactcctcatcctggtgtgaacatgaagaagtatcctgactacacgtacaagctcgacacagatcgtcgcatgatcgtcgatctgtatgtgctagaggagtttcagtttcgacgc

SEQ ID NO: 35 - DNA, 133 bp, Synthetic construct
  source 1..133: mol_type = other DNA
tgtctcactaggtcctctcactcagcgctcagtagcgtcgatgatagactgctctgtgtacgagtgcttgaacgctactggtgacacgtgcacatgtggctagagctcgagataggagtttcagtttcgacgc

SEQ ID NO: 36 - DNA, 133 bp, Synthetic construct
  source 1..133: mol_type = other DNA
tgtctcactaggtcctctcactcacaactctgtagacaagtgcttgtctcagacagctatgaacagcttcactcacttcttagacaacagcttcgacggacatccaggatgagaggagtttcagtttcgacgc

SEQ ID NO: 37 - DNA, 133 bp, Synthetic construct
  source 1..133: mol_type = other DNA
tgtctcactaggtcctctcactctaggcttctagcacacgtgcatgactcgagcagctgtcaagtgcttagacggactctagagtcatgagatcactcgagcgtgctctctacaggagtttcagtttcgacgc

SEQ ID NO: 38 - DNA, 133 bp, Synthetic construct
  source 1..133: mol_type = other DNA
tgtctcactaggtcctctcatgcactgcttctctgtcagcaagttgtctcctatctcaggtcatgagttgtcatgacgagtacgtcgagacatcgtcgctagctcatatccgaaggagtttcagtttcgacgc

SEQ ID NO: 39 - DNA, 133 bp, Synthetic construct
  source 1..133: mol_type = other DNA
tgtctcactaggtcctctcatgcagcgctcagctggtccgatgaagactcgtgtctctacgtcgtagatagagcgcttctgtgatcgcacactcgagccagtgaactctgtcaaggagtttcagtttcgacgc

SEQ ID NO: 40 - DNA, 133 bp, Synthetic construct
  source 1..133: mol_type = other DNA
tgtctcactaggtcctctcatgcgtggctcaggtgatccgtgcataccatatgtctagcacacgacacagactccgtctggagtcagcagtcagactcatcactacggtatgaaggagtttcagtttcgacgc

SEQ ID NO: 41 - DNA, 133 bp, Synthetic construct
  source 1..133: mol_type = other DNA
tgtctcactaggtcctctcatgctcgagacagctacgaacatacagactcatgctggtgcgtcgttgctcacatgcagactgcgtcgcagattgagtggctgctctccacatgaggagtttcagtttcgacgc

SEQ ID NO: 42 - DNA, 133 bp, Synthetic construct
  source 1..133: mol_type = other DNA
tgtctcactaggtcctctcagagctccgactggagcgagcaacatagacgtagtgcacttcagcagtctagcatctggagtacgtctgcagttacacggacatcctagctgataggagtttcagtttcgacgc

SEQ ID NO: 43 - DNA, 133 bp, Synthetic construct
  source 1..133: mol_type = other DNA
tgtctcactaggtcctctcagagcatctatctgtcgtcatgagatcactcgagtgcactcgtcgatgatcgctctagtgcacttcagcagtctcacattctcagcgtcgttcgaggagtttcagtttcgacgc

SEQ ID NO: 44 - DNA, 133 bp, Synthetic construct
  source 1..133: mol_type = other DNA
tgtctcactaggtcctctcagagacgatgtgcacttgacgagattgtagcgctcagctggtccgatgaagctcgatctgcactacagcagatagactcagctactcggctatgaggagtttcagtttcgacgc

SEQ ID NO: 45 - DNA, 133 bp, Synthetic construct
  source 1..133: mol_type = other DNA
tgtctcactaggtcctctcagagtcgacgtgcactatccgatcttcgacggactctagcacaacagcttgtctcagacagctacgaacatgaagaagtagtgtgcagtactgcaggagtttcagtttcgacgc

SEQ ID NO: 46 - DNA, 133 bp, Synthetic construct
  source 1..133: mol_type = other DNA
tgtctcactaggtcctctcagcactcgagcagctggtccgtcactgatcggagtgcacttcagcagtctagcattagtgcactgtgcgagtctaccattctactatgcgacataggagtttcagtttcgacgc

SEQ ID NO: 47 - DNA, 133 bp, Synthetic construct
  source 1..133: mol_type = other DNA
tgtctcactaggtcctctcagcacatatgcagatcgtcatgagatcactcagcctgtagacgcgtgcacatgtggctcacgtgatcgcacacgatagcgctagtagtcgtgacaggagtttcagtttcgacgc

SEQ ID NO: 48 - DNA, 133 bp, Synthetic construct
  source 1..133: mol_type = other DNA
tgtctcactaggtcctctcagcaacggtctctctatagcgagtcagactcatcctgactacaacagcttcactcctactgctagtccgatgaagactcacatcttcgagagctaggagtttcagtttcgacgc

SEQ ID NO: 49 - DNA, 133 bp, Synthetic construct
  source 1..133: mol_type = other DNA
tgtctcactaggtcctctcagcatcgactcagctgacaacagcttagacggactctagctcgcgaagttgaacgtgcgagtacgtctgcagttcgacgtgcactgtcgtcactaggagtttcagtttcgacgc

SEQ ID NO: 50 - DNA, 133 bp, Synthetic construct
  source 1..133: mol_type = other DNA
tgtctcactaggtcctctcaatgactgcttcttgtatcgcaacatcgctcgagtgcacttagcgagattcgcatgagtgcactcgacgaagtgatagcgctgcagagcgtgtaaggagtttcagtttcgacgc

SEQ ID NO: 51 - DNA, 133 bp, Synthetic construct
  source 1..133: mol_type = other DNA
tgtctcactaggtcctctcaatgcatacgcagctaacaacagcttgtctcagacagctacgaacagcttaccatacgtgcacttcagcagtctagagcgctctcacatcatgtaggagtttcagtttcgacgc

SEQ ID NO: 52 - DNA, 133 bp, Synthetic construct
  source 1..133: mol_type = other DNA
tgtctcactaggtcctctcaatgacgctctctagcgtccgtatcgatagcgctctgtgtcgtgcacactgatagcagtgcacttcgcgacacagactcacgagctacgtaagtaggagtttcagtttcgacgc

SEQ ID NO: 53 - DNA, 133 bp, Synthetic construct
  source 1..133: mol_type = other DNA
tgtctcactaggtcctctcaatgtcggagcagatcgtcgcatcttagcattctctggagtcacgtgcacatgtggctagaagcacacgagcataccatctagatgtaagtgagaggagtttcagtttcgacgc

SEQ ID NO: 54 - DNA, 133 bp, Synthetic construct
  source 1..133: mol_type = other DNA
tgtctcactaggtcctctcaagactcacgcagatcgtcatgagatcactcgagtgcacttacgcacactcgtcggtcctggtgacaacagcttgttcgagagtggaggatctcaggagtttcagtttcgacgc

SEQ ID NO: 55 - DNA, 133 bp, Synthetic construct
  source 1..133: mol_type = other DNA
tgtctcactaggtcctctcaagacatgactgcactgtccgttgctagagcgctcagctgtaggcaagtgatacgtagtgcactacacgatgatcgtcgctaacgtgcctgcataggagtttcagtttcgacgc

SEQ ID NO: 56 - DNA, 133 bp, Synthetic construct
  source 1..133: mol_type = other DNA
tgtctcactaggtcctctcaagaacgatgctggctgtcatgagatcgtaggcttctgtgatcgcaacatcgacgcattgcacttcagcagtctagcattaggtaacgtcacgaaggagtttcagtttcgacgc

SEQ ID NO: 57 - DNA, 133 bp, Synthetic construct
  source 1..133: mol_type = other DNA
tgtctcactaggtcctctcaagataggctcagctggtccgatgaagactcctcctgactgtccgatgatagtcggagtgcacttcagcagtctagagcgctcatagaatgacgaggagtttcagtttcgacgc

SEQ ID NO: 58 - DNA, 133 bp, Synthetic construct
  source 1..133: mol_type = other DNA
tgtctcactaggtcctctcaagcctccgtctgtgttaggcatactagactcagtgcactgtccgttgctagagcgcttctgtgatcgcacacagactcgacgtcacgtctatgaggagtttcagtttcgacgc

SEQ ID NO: 59 - DNA, 133 bp, Synthetic construct
  source 1..133: mol_type = other DNA
tgtctcactaggtcctctcaagccattctcagctatagacagcttgttcgagactgtgagtcgcaagatcgtagtcttgcgctgctgctgctgctgctgctagcgcatgcctcaggagtttcagtttcgacgc

SEQ ID NO: 60 - DNA, 133 bp, Synthetic construct
  source 1..133: mol_type = other DNA
tgtctcactaggtcctctcaagcgtctgactgacagtgtagtcagatgtgagtgtagtgactgagagactagaggatgattcacgaacaacttcttgctcttcagctactatcaggagtttcagtttcgacgc

SEQ ID NO: 61 - DNA, 133 bp, Synthetic construct
  source 1..133: mol_type = other DNA
tgtctcactaggtcctctcaagctacgtaagcagaatgcactgactagatctcacagactctcaggtggcttacctgctccgagtaacagactcgtaggctacgtcgactgagaggagtttcagtttcgacgc

SEQ ID NO: 62 - DNA, 133 bp, Synthetic construct
  source 1..133: mol_type = other DNA
tgtctcactaggtcctctcacacatgtcgacttgtctgcgagactgccagcacctaagatcgtaggtataggaggcagatgtcgagactcgagcagcagaggaggtatacacgaggagtttcagtttcgacgc

SEQ ID NO: 63 - DNA, 133 bp, Synthetic construct
  source 1..133: mol_type = other DNA
tgtctcactaggtcctctcacacctggattagagtgattgcgtgatgagccgaagtgatacttcagagacactccacactagactatgctactagagctcagagagatcggagaggagtttcagtttcgacgc

SEQ ID NO: 64 - DNA, 133 bp, Synthetic construct
  source 1..133: mol_type = other DNA
tgtctcactaggtcctctcacacatcagtacactcagtctaatctgatcggacgtagcttaggatcgttcagattctagtgtgtctcattcgatctctcacgtgtacagcagcaggagtttcagtttcgacgc

SEQ ID NO: 65 - DNA, 133 bp, Synthetic construct
  source 1..133: mol_type = other DNA
cggaggcattgagtacgatggcagtggtggtggatagactcgtagatgtccgttgcgtctgcacttgcagacattaccagcgtacaacttgcgagctcgcttacgacagagagtctgtgtgaagcggtactca

SEQ ID NO: 66 - DNA, 133 bp, Synthetic construct
  source 1..133: mol_type = other DNA
cggaggcattgagtacgatggcagtggtggtggatagactcgtagattactgaagacgtatcctactgactctctgtcacgcaacaacttgcagacatgctcagcatacacgttctgtgtgaagcggtactca

SEQ ID NO: 67 - DNA, 133 bp, Synthetic construct
  source 1..133: mol_type = other DNA
cggaggcattgagtacgatggcagtggtggtggatagactcgtagattactcgtctatggtcatcctgactacgtcgacaatggtcgtcctggagacggctatcgtcatgtgctctgtgtgaagcggtactca

SEQ ID NO: 68 - DNA, 133 bp, Synthetic construct
  source 1..133: mol_type = other DNA
cggaggcattgagtacgatggcagtggtggtggatagactcgtagatgcttgtagacgtacaagctctacgctcagaagaatgatcctacagacatcggctgcttctgacgcatctgtgtgaagcggtactca

SEQ ID NO: 69 - DNA, 133 bp, Synthetic construct
  source 1..133: mol_type = other DNA
cggaggcattgagtacgatggcagtggtggtggatagactcgtagatagataggctacagtcagctctgagctcagaacagcacgtacttgcatcctcactagcgagtgtgtatctgtgtgaagcggtactca

SEQ ID NO: 70 - DNA, 133 bp, Synthetic construct
  source 1..133: mol_type = other DNA
cggaggcattgagtacgatggcagtggtggtggatagactcgtagatgcttcacacgcagtcatcagatcttcgtacacacgtgtggtgctggaccatactcattcaagaagatctgtgtgaagcggtactca

SEQ ID NO: 71 - DNA, 133 bp, Synthetic construct
  source 1..133: mol_type = other DNA
cggaggcattgagtacgatggtccattacgcggcaacagagctgactctccatgctacagtctagctgtgcctcagaacatgcgtcgctctgactacgacttactgccgatcatctgtgtgaagcggtactca

SEQ ID NO: 72 - DNA, 133 bp, Synthetic construct
  source 1..133: mol_type = other DNA
cggaggcattgagtacgatgcgacattaggctacagtcctgtctacgcattgttcacgaacagtgcaggctacttgtgtccgagtcgtgtctgcttagactctgagaacgtgatctgtgtgaagcggtactca

SEQ ID NO: 73 - DNA, 133 bp, Synthetic construct
  source 1..133: mol_type = other DNA
cggaggcattgagtacgatggagcattacgcagcaatcacttgcgacacgtaggctacacgagaggacgctgtgcagagactcgtcatcctgagcctcagtcacgtgcgttgctctgtgtgaagcggtactca

SEQ ID NO: 74 - DNA, 133 bp, Synthetic construct
  source 1..133: mol_type = other DNA
cggaggcattgagtacgatgcagctctcgacacgacgttagctggctgtgtacgatcgacgtctgctgacgctcagaacacgatagacttgcactcatagttcgcgaatctactctgtgtgaagcggtactca

SEQ ID NO: 75 - DNA, 133 bp, Synthetic construct
  source 1..133: mol_type = other DNA
cggaggcattgagtacgatgacgctcagatgccgaatctgtcaggctagctaccgcatggtctgccagtctctcagacactgccgtctgcagtagacgagttctatcagccagtctgtgtgaagcggtactca

SEQ ID NO: 76 - DNA, 133 bp, Synthetic construct
  source 1..133: mol_type = other DNA
cggaggcattgagtacgatgctcctctcggatgcatacgagcaggagctctagacacgagtcacttgccgatcgtcgtaccgatcactactggagtcgagttcggtctgcatctctgtgtgaagcggtactca

SEQ ID NO: 77 - DNA, 133 bp, Synthetic construct
  source 1..133: mol_type = other DNA
cggaggcattgagtacgatgcagactagaagtgcacgtgtgcaggctacttcaagtgcatcgctacaggcttagtacagtgcacgactgcagctactcctcatctcatgcatgtctgtgtgaagcggtactca

SEQ ID NO: 78 - DNA, 133 bp, Synthetic construct
  source 1..133: mol_type = other DNA
cggaggcattgagtacgatgcagcattacagacgtacagtgcagcagactagaagaatgatcctacagtctctccatgctagtatctgtctgcgtcatctcgtacagcagtcttctgtgtgaagcggtactca

SEQ ID NO: 79 - DNA, 133 bp, Synthetic construct
  source 1..133: mol_type = other DNA
cggaggcattgagtacgatggctagctcgagtgcacgtgaggacgctgtgtacacagcatcactgtctgcttagtcgcaggcagtctgcctgtctacgctctgcagcctgtactctgtgtgaagcggtactca

SEQ ID NO: 80 - DNA, 133 bp, Synthetic construct
  source 1..133: mol_type = other DNA
cggaggcattgagtacgatggcatcgtcgtgccgtcgagagcagatccattcagatgcacgtacttgcatcctctcacacgcatagctacaggcttagctccatgtaacggtatctgtgtgaagcggtactca

SEQ ID NO: 81 - DNA, 133 bp, Synthetic construct
  source 1..133: mol_type = other DNA
cggaggcattgagtacgatgactacgtgacacgcagtcgctgaggtgactcagtgactatacatcgcaagcgtgcatgctagtctctgtctgacactctgccgttcagtatcttctgtgtgaagcggtactca

SEQ ID NO: 82 - DNA, 133 bp, Synthetic construct
  source 1..133: mol_type = other DNA
cggaggcattgagtacgatggctagctagagtcgagtgactctggctgtgtacacagcatcaacttgcagcagtagaagtgcacgagtgctggactcctgcgactgcatgtcgtctgtgtgaagcggtactca

SEQ ID NO: 83 - DNA, 133 bp, Synthetic construct
  source 1..133: mol_type = other DNA
cggaggcattgagtacgatgcgtctcagaagtctctacgctgcagctagctacagacgtcgtctactgacgacgcatgctacagcttagctgtgcacgtgctagcactgctgctctgtgtgaagcggtactca

SEQ ID NO: 84 - DNA, 133 bp, Synthetic construct
  source 1..133: mol_type = other DNA
cggaggcattgagtacgatggctagctcaagaatggtcgtctctctaacgtgatgacgatagacttgcgactcctgtagaatggtcgtctctagatcgtgctctgtagtaacttctgtgtgaagcggtactca

SEQ ID NO: 85 - DNA, 133 bp, Synthetic construct
  source 1..133: mol_type = other DNA
cggaggcattgagtacgatgactctctgtgctacagtcgtgctgcagctcagagatgcaacgacttgcatcctctcacacgcagtctgcctgtctctcgagcatcacgtggtgtctgtgtgaagcggtactca

SEQ ID NO: 86 - DNA, 133 bp, Synthetic construct
  source 1..133: mol_type = other DNA
cggaggcattgagtacgatggcatcgtcgtgccgtcgagagcagatccattcagatgcacgtacttgcatcctctcaagtgcatcaacttgcctgcatgagcagtgaagaatgtctgtgtgaagcggtactca

SEQ ID NO: 87 - DNA, 133 bp, Synthetic construct
  source 1..133: mol_type = other DNA
cggaggcattgagtacgatgactacgtcggctagtatcgagcagacgctcagagcacgagtcagctctgagacgtagcacatgcgagtgctgtagacggagcagtagtgcatctctgtgtgaagcggtactca

SEQ ID NO: 88 - DNA, 133 bp, Synthetic construct
  source 1..133: mol_type = other DNA
cggaggcattgagtacgatggctagctacgcacgagtgctatctctgcattgttaccgagtcgtgtctgagctctaggctacaatctagctggcttaggagcgttcatgtacttctgtgtgaagcggtactca

SEQ ID NO: 89 - DNA, 133 bp, Synthetic construct
  source 1..133: mol_type = other DNA
cggaggcattgagtacgatgactctctcgtacgcagtcgtctctagacattaggctagtgtgtgtctggactcgtcacgcatggtctgcctgtctctcgcatgacattagcgatctgtgtgaagcggtactca

SEQ ID NO: 90 - DNA, 133 bp, Synthetic construct
  source 1..133: mol_type = other DNA
cggaggcattgagtacgatggctacttgtgtccgaacaacttgccgttcgtcgtgcatgcgagtgcaggcttagtacagtgcacgtacttgcagtcatgcatcggatgcatcatctgtgtgaagcggtactca

SEQ ID NO: 91 - DNA, 133 bp, Synthetic construct
  source 1..133: mol_type = other DNA
cggaggcattgagtacgatggagctcagatctatgtaggagctgatcctctgtcacgcacgtacttgcagcacgtcggctagttcaactctgagcacggcactacagctagactctgtgtgaagcggtactca

SEQ ID NO: 92 - DNA, 133 bp, Synthetic construct
  source 1..133: mol_type = other DNA
cggaggcattgagtacgatgacgctcagacagcgacgtagctctctgctcagaacagcacgtacttgcactctcagagcacgatcaagccaggagtcggcatctcatgctagttctgtgtgaagcggtactca

SEQ ID NO: 93 - DNA, 133 bp, Synthetic construct
  source 1..133: mol_type = other DNA
cggaggcattgagtacgatggcttagtcgcaggcagtcatcctgactctctgtcacgcataggagctgactctctcggctacacgttagctggagctcatggctagttcgtactctgtgtgaagcggtactca

SEQ ID NO: 94 - DNA, 133 bp, Synthetic construct
  source 1..133: mol_type = other DNA
cggaggcattgagtacgatggctacttgttctcgtcgtctagaggctacttcacacatgcgtgtgcagacacattagagagcatcaactctggctagcatggagctatcacactctgtgtgaagcggtactca

SEQ ID NO: 95 - DNA, 133 bp, Synthetic construct
  source 1..133: mol_type = other DNA
cggaggcattgagtacgatggacacgtaggctacagtctagctggctagctcatgacgagtgactctggcttagtgtacagcaacactacagctggtgatgctcctccgagactctgtgtgaagcggtactca

SEQ ID NO: 96 - DNA, 133 bp, Synthetic construct
  source 1..133: mol_type = other DNA
cggaggcattgagtacgatgctgagccatgctacatcgactctgagctcgtacgtcgcagtctgcctgacgctcagaagaatgatcctacaggcttagatgtcgacagcgctatctgtgtgaagcggtactca

SEQ ID NO: 97 - DNA, 133 bp, Synthetic construct
  source 1..133: mol_type = other DNA
cggaggcattgagtacgatgagtcattactgccgtacactgtctatcctcagagctcgtatcctacagctccattaggctacacgttagcaggctactagacattgcgaggagtctgtgtgaagcggtactca

SEQ ID NO: 98 - DNA, 133 bp, Synthetic construct
  source 1..133: mol_type = other DNA
cggaggcattgagtacgatgtgccattgtgcaatgtcaacttgcatcctctcaagtgcatcaacttgcctccattactgagcagtcagacaggagcatagatcaatggatacgtctgtgtgaagcggtactca

SEQ ID NO: 99 - DNA, 133 bp, Synthetic construct
  source 1..133: mol_type = other DNA
cggaggcattgagtacgatggctagctcgagtgcaacactacaggcttagtcgcacatgtcaacttgcagtcatgatgctagttcaactctggagacgagatgatagacaatgtctgtgtgaagcggtactca

SEQ ID NO: 100 - DNA, 133 bp, Synthetic construct
  source 1..133: mol_type = other DNA
cggaggcattgagtacgatggctacttcagcgcgtcgagagcagtctctcagacaccgatgactacagtctactagagcactacgtacttgcatctcgagaacaagctcttgttctgtgtgaagcggtactca

SEQ ID NO: 101 - DNA, 133 bp, Synthetic construct
  source 1..133: mol_type = other DNA
cggaggcattgagtacgatgagacattagtgccgtacaacttgcagctcgtgagctagttacgtgctggagacgtcgagacgttgaacttgcgacctcagctgtcgtactctatctgtgtgaagcggtactca

SEQ ID NO: 102 - DNA, 133 bp, Synthetic construct
  source 1..133: mol_type = other DNA
cggaggcattgagtacgatggcactcagatgccgtcgttgtctgctgctcagaagtgcatcaacttgcatcctctcacacgcagtcagacaggaccatagcctggtagctagatctgtgtgaagcggtactca

SEQ ID NO: 103 - DNA, 133 bp, Synthetic construct
  source 1..133: mol_type = other DNA
cggaggcattgagtacgatgatcctctgtgatcgacgagagtgcgctactgtggatgcacgttgttgcatcctcagaagacgtacagtgctgcagacgagcatgcgagtcacatctgtgtgaagcggtactca

SEQ ID NO: 104 - DNA, 133 bp, Synthetic construct
  source 1..133: mol_type = other DNA
cggaggcattgagtacgatggctagctcgcacgcagtctgccagagttcgtcgcacatgtcaacttgcctgcattcagcagcatagtgtctggcatcgagctcacgatgatcgtctgtgtgaagcggtactca

SEQ ID NO: 105 - DNA, 133 bp, Synthetic construct
  source 1..133: mol_type = other DNA
cggaggcattgagtacgatgagcctctcgtcacgagtcgctcatgcagtggcaagtgtgatcgtgtcgctcctctcagtgctgcatgtcatgcgaactcacactctgactatctctgtgtgaagcggtactca

SEQ ID NO: 106 - DNA, 133 bp, Synthetic construct
  source 1..133: mol_type = other DNA
cggaggcattgagtacgatgtcggagctggtcgtatacacggagagcgcaatcatctagtcagaggctagctgctagagcgctgaggtatcggtgagccactcggtctgagattctgtgtgaagcggtactca

SEQ ID NO: 107 - DNA, 133 bp, Synthetic construct
  source 1..133: mol_type = other DNA
cggaggcattgagtacgatgtcagtctgccaccgtatcctcctcgacgtatgactatagcactagtcacatgtgtcagctctctacctactcagagtgcacgtgtagacgtagtctgtgtgaagcggtactca

SEQ ID NO: 108 - DNA, 133 bp, Synthetic construct
  source 1..133: mol_type = other DNA
cggaggcattgagtacgatgagagagtctagccgacgactaacgacaagcctgtgcactagcgtcatcactgtagtggagcgatcagaggtatcgtcagcgctacattcgtgctctgtgtgaagcggtactca

SEQ ID NO: 109 - DNA, 133 bp, Synthetic construct
  source 1..133: mol_type = other DNA
cggaggcattgagtacgatgtgtgacgcgtagacgtgctactgatcttgtactcactgttgtctgacgcgagcatgcgagcacgaggcttcaacgactcatcgagacgcatcatctgtgtgaagcggtactca

SEQ ID NO: 110 - DNA, 133 bp, Synthetic construct
  source 1..133: mol_type = other DNA
cggaggcattgagtacgatgtgtatctgctctcatagtgagagttaccaggtctcgtgttgcgagagccagtctctgtgctgcagagattcaagtagacatgcagcaacatcttctgtgtgaagcggtactca

(19) United States
(12) Patent Application Publication
(10) Pub. No.: US 2026/0308674 A1
(43) Pub. Date: Oct. 8, 2026

(54) MOLECULAR ENCRYPTION OF DNA-ENCODED DATA IN MICROCOMPARTMENTS

(71) Applicant: TECHNISCHE UNIVERSITEIT EINDHOVEN, EINDHOVEN (NL)

(72) Inventors: Bastiaan Wilhelmus Albertus BÖGELS, EINDHOVEN (NL); Tom Franciscus Antonius DE GREEF, EINDHOVEN (NL)

(21) Appl. No.: 19/482,213

(22) PCT Filed: Feb. 20, 2025
(86) PCT No.: PCT/NL2025/050082

§ 371 Date: Nov. 6, 2025

(30) Foreign Application Priority Data
Feb. 20, 2024 (NL) 2037071

(51) Int. Cl. C12Q 1/686; G06F 21/60

(52) U.S. Cl. CPC C12Q 1/686; G06F 21/602

(57) ABSTRACT

Highly advantageous means and methods are provided to protect data, i.e. through physical molecular encryption of nucleic-acid encoded data. Briefly, digital data, i.e. information is first encoded and designed into nucleic acid-encoded data; based on this nucleic acid-encoded data polynucleotide fragments are made. The nucleic acid encoded data is molecularly encrypted, i.e. molecularly locked. Molecularly locked means that access to the information in the nucleic acid encoded data, once converted into nucleic acid molecules, is blocked, which lockage can be unlocked by providing a specific unlocker, a molecule, that functions as a key to gain access to the nucleic acid.

### INTRODUCTION

There is an immense and continuously growing need for data storage solutions that are durable and safe. Without such storage solution, personal or sensitive data may be accessed by an unauthorized party, which is highly undesirable. Conventional methods use magnetic or optical storage solutions such as hard-drives. Safe storage thereof is often achieved by physically restricting access or by digital encryption. DNA data storage has several advantages compared to magnetic or optical data storage, such as extremely high data densities, high stability to allow for durable storage, and limited energy needs. Considerable effort has been devoted to developing efficient encoding algorithms and methods for DNA storage and retrieval. However, a complete data storage solution also requires the stored information to be protected from unwanted data access. It is evident that DNA storage can be used to efficiently store data, but there is a clear need to also safely store such data.

Hence, there is a need in the art to provide for improved means and methods for DNA-encoded data that allow for safe and efficient storage of data.

### SUMMARY OF THE INVENTION

The present invention now provides for means and methods of molecular data encryption of DNA-encoded data that is compatible with random access by polymerase chain reaction (PCR) and reduces the impact on data densities incurred by obfuscation-based methods.

Since DNA-encoded data is typically stored across several polynucleotides, in the present invention, it was advantageously realized that by selectively preventing the amplification of at least a subset of these molecules unwanted data access can be prevented during PCR-based (random) access, as the data set cannot be completed. Selective repression of PCR can be achieved by preferential binding of a locker oligonucleotide that outcompetes a PCR primer and subsequently prevents polymerase extension. Locker strands are preferably and advantageously co-compartmentalized with the data-encoding DNA using e.g. biotinylated strands bound to biotin-binding agents. Using toehold-mediated strand displacement, the locker, i.e. blocking strand, which is unbiotinylated, can be removed from a compartmentalized file by an unlocker or password strand. Consequently, by removal of the locker strand which requires a specific unlocker, i.e. an oligonucleotide with a defined sequence, PCR-based amplification is restored, therewith providing for password-mediated access in the form of a molecule to the entire DNA-encoded digital data file.

### FIGURES

FIG. 1a: Locker strand concentration dependent blocking of PCR. Schematic outline of experiment to measure effects of locker strand on PCR efficiency. Two templates (said templates being dsDNA polynucleotide fragments according to the invention) are provided. Template A; comprising sequences A1, A2 which are complementary sequences and of which one comprises the locker target (T) sequence and the other is complementary thereto. Template B comprising sequences B1, B2, which are complementary sequences lacking a Locker target (T), were subjected to PCR amplification in the presence of increasing locker (L) concentrations and universal primers (UFW+URV, which bind to the 5′ ends of the A1, A2, B1 or B2 sequences). Obtained amplified product was subsequently purified and relative concentrations of templates A and B after amplification using template A and B specific primers (AFW+ARV and BFW+BRV, respectively), were determined using qPCR.

FIG. 1b: Measured relative concentrations template A (left bar of each set) and B (right bar of each set) after initial PCR. Threshold cycle (Ct) values were determined in three individually PCR amplified mixtures for templates A and B. Statistically significant differences in relative concentrations of the two dsDNA templates were observed for locker concentrations larger than 50 nM using a two-sided Student's t-test. The bars denote the mean Ct-values for three experiments; the points represent individual experiments. Sequences described are listed in Table 1. Measured Ct values are listed in Table 2.

FIG. 2a: Unlocking proteinosome-localized PCR templates using DNA strand displacement. Schematic outline of unlocking of proteinosome-localized templates. Both dsDNA template A comprising sequence A3, A4 which are complementary sequences and of which one comprises the locker target (T) sequence and the other is complementary thereto, and template B comprising sequence B3, B4 which are complementary sequences lacking a Locker target (T), the Locker Complex (LC) comprising the locker strand (L1) hybridized to the biotin-labeled complementary locker anchor strand (L2), are colocalized in proteinosomes by means of a protein, with one or more biotin binding domains, i.e. a biotin-binding protein (BB). The proteinosomes further comprise an optional magnetitic bead (M). The password (P), to which hereafter interchangeably may also be referred to as ‘unlocker’, ‘unlocker strand’, ‘password’ or ‘password strand’ is complementary to the locker strand (L1) and can hybridize to displace the locker from the proteinosomes using toehold-mediated DNA strand displacement (DSD) and optional washing. Unlocked and locked proteinosome-localized templates were subjected to PCR amplification with universal primers (UFW which binds to the 5′ ends of the A3 B3, and URV which binds downstream of the poly-T stretch of the A4, B4 sequences) and the difference in relative concentrations of the templates is determined using qPCR using template A and B specific primers (AFW+ARV and BFW+BRV, respectively).

FIG. 2b: Differences in relative concentrations of the two templates after locked and unlocked PCR. After amplification and purification of PCR reaction mixtures the relative concentrations of templates A and B were determined using qPCR and the difference in Ct values (ΔCt) was determined for each replicate. A statistically significant difference of 1.0 cycle was observed between the locked (left) and unlocked (right) states using a two-sided Student's t-test (p=0.026). The horizontal lines denote the mean ΔCt for three experiments; the points represent individual experiments. Sequences described are listed in Table 3. Measured Ct-values are reported in Table 4.

FIG. 3a: Colocalization of locker and template strands in biotin-binding protein containing proteinosomes. Schematic representation proteinosomes used for colocalization experiments. A fluorescently labelled (denoted by a star) and biotinylated template strand (T) was localized inside biotin-binding protein containing proteinosomes together with fluorescently labelled locker strand (L) comprised in a locker complex. Template strands (T) were labelled with Cy5 fluorophores and locker strands (L) with Cy3 allowing for visualization of both complexes. Proteinosome membranes were labelled with DyLight405. Proteinosomes were then imaged using confocal microscopy.

FIG. 3b: Confocal micrographs of template strands and locker complex containing proteinosomes. Shown is the colocalization of labelled locker complex (L; middle) and labelled template strands (T; right) together with proteinosome membranes (Membrane; left) indicating that DNA is internalized inside the proteinosomes. Scale bars indicate 250 micrometers.

FIG. 4a: Toehold-mediated strand displacement of locker strand from proteinosomes. Schematic representation of proteinosomes used for locker displacement experiments. A fluorescently labelled (denoted by a star) and biotinylated template strands were localized inside biotin-binding protein containing proteinosomes together with fluorescently labelled locker complex. Template strands were labelled with Cy5 fluorophores and locker complexes with Cy3 allowing for visualization of both complexes. Proteinosome membranes were labelled with DyLight405. After localization, password strand (P) was added to initiate toehold-mediated strand displacement of the fluorescent locker strand. After optional washing to remove non-localized DNA, proteinosomes were imaged using confocal microscopy.

FIG. 4b: Confocal micrographs of template and locker complex containing proteinosomes. Shown is the colocalization of template strands (T; right) together with proteinosome membranes (Membrane; left) indicating that template strands are internalized inside the proteinosomes. Due to the addition of an unlocker and by optional washing, it is shown that locker strands (L; middle) have been successfully displaced. Scale bars indicate 250 micrometers.

FIG. 5a: Simulating locked PCR on data-encoding DNA. Schematic representation of the stochastic model used to simulate the effects of varying the fraction of blocked strands (B, comprising a Locker target) and the effectiveness of PCR blocking. To simulate blocked PCR, 1000 template strands were initialized, a fraction of which were set to be blocked (B) and wherein the remainder of 1000 template strands comprise non-blocked (NB, lacking a Locker target) template strands. For every template strand, 20 PCR cycles are simulated, during which the doubling chance parameter (PD) controls the odds of a template strand being effectively doubled during PCR, simulating the effects of blocked PCR. Sequencing is simulated as a random sampling from the total pool of amplified template strands to a fixed average coverage of 10×.

FIG. 5b: Heatmap of simulated dropout for varying fractions of blocked strand (B) and doubling chance (PD). Simulated coverage distributions are shown in FIG. 5c.

FIG. 5c: Coverage distribution for simulated file-level DNA locking for varying PD and fractions of blocked strands.

FIG. 6: Flowchart outlining means and methods in accordance with to the invention.

FIG. 7. Schematic depicting “bead-based” unlocking. Templates A (a) and B (b) are mixed with a locker strand (sideways T-shaped object) to form a locked template solution. The A double stranded template comprises a locker target sequence. Unlocking is achieved by addition of a beads bound with an unlocker, also referred to as password beads, formed through the interaction between biotinylated password strand and streptavidin-coated magnetic particles. Locked and unlocked samples are PCR amplified using PCR primers and relative concentrations of A and B are subsequently determined using qPCR to determine the difference in Ct (ΔCt). The ΔCt (A-B) comparing locked vs. unlocked, is expected to be higher in the locked scenario vs. the unlocked scenario, indicative of PCR-amplification being reduced by the presence of the locker.

FIG. 8. Experimental results of an unlocking experiment as depicted FIG. 7. Differences in relative concentrations of the two templates after PCR for various experimental conditions. After amplification and purification of PCR reaction mixtures the relative concentrations of templates A and B were determined using qPCR (Experimental section) and the difference in Ct values (ΔCt) was determined for each replicate. Beads with passwords, i.e. unlocked, indicate and experiment in which the locked mixture was incubated with unlocker, i.e. beads bound with a password strand. “No locker added” indicates a positive control which contained only templates A and B without any locker present in the mixture. “Beads with random sequence” is an experiment in which the locked solution was incubated with beads bound with an oligonucleotide, a DNA strand, which was not complementary with the locker strand, i.e. an incorrect unlocker. “No beads added” represents a negative control of the locked solution which was not subjected to unlocking. Statistical significance between various samples is indicated above the lines connecting various samples and was determined using a One-way ANOVA test, The horizontal lines denote the mean ΔCt for three experiments; the points represent individual experiments. The ΔCt for beads with passwords and no locker added was about −2 on average. The ΔCt for beads with an incorrect unlocker or with no unlocker added, was about 5. The difference in ΔCt between unlocked and locked or incorrect unlocker is about 7, which difference was statistically different, i.e. about 6.9 and 7.2 respectively. This shows that PCR-amplification of polynucleotide fragments with locker target sequences can be highly efficiently blocked with a locker.

FIG. 9: Repeatable locking and unlocking of physical molecular encrypted nucleic acid-encoded data according to the invention. a. Cartoon of data encoding using DNA-GUARD. A text file of 549 bytes was encoded onto 42 DNA strands (File 1). All strands share two universal primer-binding sites, FP and RP, and an internal addressing structure to reorder the data after sequencing. Furthermore, 6 out of the 42 strands contain the additional PW region, allowing these strands to be locked by preventing their amplification. b. Cartoon describing locking and unlocking of DNA-encoded data. File 1 can be locked to prevent decoding by adding locker strand L1. PCR of the file using universal primers U_{FW} and U_{RV} in the presence L1 greatly reduces amplification of lockable strands, which results in an incomplete, and therefore unreadable, file after decoding. To unlock File 1, molecular passwords, consisting of magnetic bead-bound PW₁ strands complementary to L₁, are added to the solution. Upon sequestration of L1 by the molecular passwords, L₁-molecular password hybrids can be removed using magnetic retrieval. Subsequently, PCR can amplify the unlocked file, and the resulting DNA can be sequenced for file decoding. c. Relative number of sequencing reads of file-encoding strands after PCR in locked state and unlocked state. An initial 1.68 nM of File 1 was amplified using 500 nM primers U_{FW} and U_{RV} in the presence of 2.5 μM locker strand L1, and a sample was kept for sequencing. The remainder was unlocked by using magnetic particle-bound PW₁. Both locked and unlocked samples were amplified using PCR before sequencing, and each strand's relative number of reads was determined (Methods). Strands with a PW region are shown in red, and strands without a PW region are shown in green. Bars denote the mean number of relative reads over 3 replicates; points indicate individual experiments. Sequences for File 1, Fw₁, Rv₁ and L₁ are given in Tables 6 and 7. d. Ratio of relative File 1 reads for lockable to non-lockable strands in locked and unlocked states. An initial 1.68 nM of File 1 was PCR amplified using 500 nM primers U_{FW} and U_{RV} in the presence of 2.5 μM locker strand L1, and a sample was kept for sequencing. The remainder was unlocked using magnetic particle-bound PW₁ and PCR amplified. Not-locked, locked, and unlocked File 1 PCR products were sequenced, and the relative number of reads was determined for each strand (Methods). The ratios of strands that contain the PW region to strands that do not contain the PW region were calculated. A statistically significant (p<0.001) 250-fold difference as determined using two-sided Welch's t-test was observed between the original File 1 solution and locked File 1, and a statistically significant (p<0.001) 440-fold difference was observed between locked and unlocked File 1 using two-sided Welch's t-test. Stars indicate statistical significance: *: p<0.05; **: p<0.01. e. Simulation used to determine functionality of DNA-GUARD as a data protection method. Reads obtained from the locked PCR of File 1 are randomly sampled to simulate an average coverage of 30 reads per strand. Decoding is attempted, and the result is recorded. These steps are repeated 1000 times to determine the percentage of times File 1 cannot be accessed once locked. f. Determining readability of File 1 for three independent experiments randomly down-sampled to 30× coverage. Experimental sequencing reads were down-sampled to an average coverage of 30 reads per strand (see Methods), and the number of sequences containing the password region with one or more reads was determined. For decoding of File 1, at least four out of six password region containing sequences have to be presented, as indicated by the horizontal line. Solid lines denote mean number of sequences not present; points indicate individual experiments, g. Percentage of tries File 1 is locked after 1000 simulated samplings. The percentage of trials File 1 could be decoded for each sequenced experiment was determined before locking, in a locked state, and after unlocking. In both unlocked states, File 1 could be decoded in 100% of the trials; in the locked state File 1 could not be decoded in 99.20+0.22% (mean±standard deviation) of the trials. Solid lines denote mean percentage of unreadable trials; points indicate individual experiments. Stars indicate statistical significance: *: p<0.05; **: p<0.01.

FIG. 10: Demonstration of a process on a DNA-encoded file.

FIG. 11: Scalability of physical molecular encryption of nucleic acid-encoded data.

FIG. 12: Comparison of an unlocker (i.e. password; PW₁) design wherein the locker comprises an inverted dT (−InvdT) with an unlocker lacking an inverted dT.

### DETAILED DESCRIPTION

Herewith, means and methods for the protection of data from unwanted access are provided. Highly advantageously, means and methods are provided for physical molecular encryption of nucleic acid-encoded data.

Conventionally, most data, such as digital data, is stored using magnetic or optical data carriers such as hard-drives, tapes, disc-drives or the like. Consequently, there is (and has been) a general need for providing safe storage solutions that can protect said data from unwanted access, e.g. when confidential data is stored. Conventional means and methods to protect from unwanted access exist, and these solutions typically rely on physical and/or digital protection. For example, physical separation provides protection by storing data in an inaccessible area, such as a safe. Digital means provide protection by implementing e.g. digital encryption algorithms which converts the data from the original state into a state that is essentially hiding the original data. In both scenarios, only authorized parties that holds the key(s), be it physical or digital, will have access to the data.

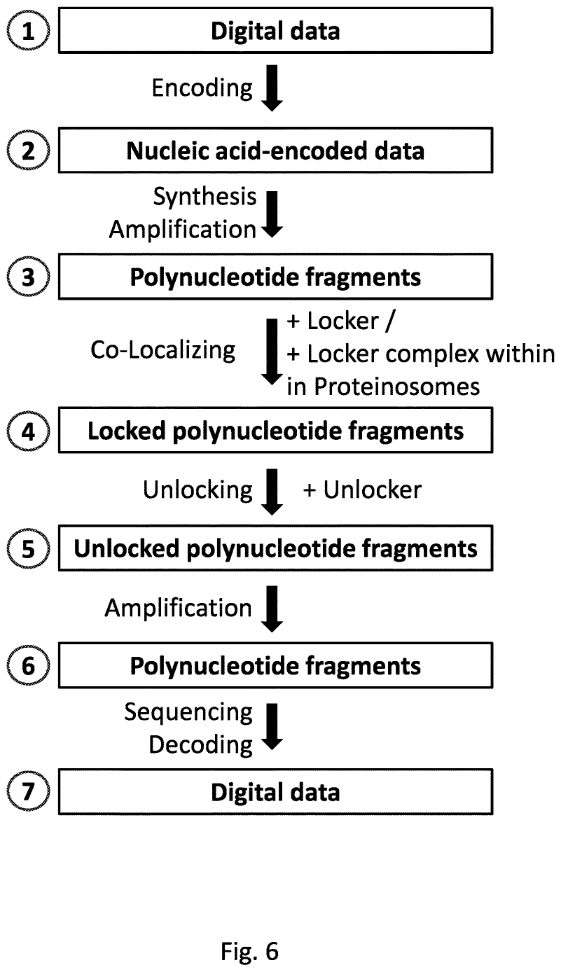

Here, highly advantageous means and methods are provided to protect data, i.e. through physical molecular encryption of nucleic-acid encoded data as further elucidated herein. Briefly, and as schematically illustrated in FIG. 6, digital data, i.e. information is first encoded and designed into nucleic acid-encoded data; based on this nucleic acid-encoded data polynucleotide fragments are made, i.e. synthesized, e.g. as doubles DNA molecules as shown in the examples (FIG. 6; step 1-2). This digital data is molecularly encrypted, molecularly locked. Molecularly locked means that access to the information in the nucleic acid code data, once converted into nucleic acid molecules, is blocked by a molecule, an oligonucleotide, i.e. a so called locker. By preparing the polynucleotide fragments and co-localizing these with the so-called locker, e.g. in a container or in proteinosome compartments (with biotin-binding protein with the locker associated therewith via a biotinylated locker anchor and with biotinylated polynucleotide fragments (FIG. 6, step 3-4)) locked polynucleotide fragments are obtained. The locker blocks amplification of the polynucleotide fragments (e.g. by conventional PCR using primers), said amplification to allow for subsequent sequencing and retrieval of the nucleic-acid encoded data. Highly advantageously, the implementation of the locker prevents PCR amplification by preferentially binding to the polynucleotide fragments, thereby outcompeting primers. Hence, retrieving sequence information from the polynucleotide fragments and therefore the nucleic acid-encoded data is physically locked. Advantageously, only authorized party can have easy access to the data stored in the polynucleotide fragments when in possession of a so-called unlocker (i.e. an oligonucleotide, either physically, or having the sequence information). The unlocker unlocks the polynucleotide fragments by interacting with the locker, resulting in removal of the locker (FIG. 6; step 5). Once the locker is removed with the unlocker, the fragments can be efficiently PCR amplified. Once amplified polynucleotide fragments are obtained, these can be sequenced and decoded to thereby retrieve the digital data (FIG. 6; step 6-7).

The physical molecular encryption of nucleic-acid encoded data is explained in further details below, as well as further embodiments thereof. When used in this specification and claims, the terms “comprises” and “comprising” and variations thereof mean that the specified features, steps or integers are included. The terms are not to be interpreted to exclude the presence of other features, steps or components.

Accordingly, herein are provided are means and methods, and a system, for physical molecular encryption of nucleic acid-encoded data. In a first embodiment, a method is provided for physical molecular encryption of nucleic acid-encoded data, the method comprising the steps:
a) providing a digital dataset;
b) encoding the digital dataset to a nucleic acid code, thereby designing a nucleic acid-encoded dataset, wherein the nucleic acid-encoded dataset comprises a plurality of polynucleotide sequences of between 50-500 nucleotides, preferably 100-250 nucleotides, more preferably 150-200 nucleotides, wherein each of the polynucleotide sequences further comprise a forward and reverse primer binding site at the ends of the polynucleotide sequences that allows for subsequent PCR amplification wherein up to 100% of the individual polynucleotide sequences comprise at least one locker target sequence wherein the locker target sequence is located such that PCR amplification is blocked when a locker is bound to the locker target sequence, optionally wherein the locker target sequence and/or the primer binding site(s) of the polynucleotide sequence is/are part of the nucleic acid-encoded dataset, and/or optionally wherein the polynucleotide sequences comprise an indexing sequence;
c) synthesizing the designed plurality of polynucleotide sequences to provide for polynucleotide fragments, wherein the polynucleotide fragments are synthesized as single stranded polynucleotide fragments and subsequently made double stranded polynucleotide fragments or synthesized as double stranded polynucleotide fragments, preferably comprising an amplification step to provide for a desired concentration of double stranded polynucleotide fragments;
d) labeling the double stranded polynucleotide fragments with biotin to therewith provide for biotin-labeled double stranded polynucleotide fragments representing the nucleic acid-encoded dataset.

In step a) of the method according to the invention a digital dataset is provided. A digital dataset is any type of information which may be typically found and/or stored on a digital device such as a computer, a phone, a hard-drive or the like. Digital data can take various forms, including text, numbers, images, audio, video, and more. For example, a text document comprising a plurality of characters that make up the words can be represented as a sequence of binary digits where each character is assigned a unique binary code. Similarly, images and videos can be broken down into pixels, with each pixel having a digital and/or binary representation. Therefore, the digital dataset can be provided as a binary file. The digital dataset can be provided as raw, unedited files i.e. the text, numbers, images, audio, video or the like are not transformed, altered or converted or otherwise. Any type of digital data can be contemplated in accordance with the invention.

In step b) of the method the digital dataset is encoded to a nucleic acid code thereby designing a nucleic acid-encoded dataset. The encoding involves the conversion of the provided digital information to a nucleic acid sequence, thereby providing a nucleic-acid encoded dataset. The nucleic acid-encoded dataset according to step b) of the invention is at this step information, e.g. a text file, which is to be subsequently used as information to prepare, e.g. synthesize, polynucleotide fragments, such as DNA fragments. It is further understood that in nature, nucleic acids contain nucleic-acid code, so-called codons/triplets, which are used for translation of nucleic acid into, amino acids to make up a protein. It is understood that the building blocks of polynucleotides, such as the four letters for DNA bases A, G, C and T, are used in accordance with the invention, for encoding, utilizing e.g. singlets, doublets, triplets, or quadruples or more consecutive bases as coding elements, wherein e.g. a binary code of a digital dataset is converted to a selected nucleic acid code.

Hence, encoding digital data into nucleic acid code involves the use of specific rules and procedures to transform data, such as digital data, from one state to another, and a corresponding decoding algorithm is used to reverse the process when necessary. In the context of encoding digital data into nucleic acid code in accordance with the invention, algorithms are used to convert data from one form to another, and back. It is understood that for the purpose of the invention, preferably the digital data which is encoded into nucleic acid-encoded data is efficient. It is advantageous to use an encoding algorithm that converts as many bits of the digital dataset to as little as possible nucleotides. Thus, the number of bits encoded on a single nucleotide is improves the data density. Of course, some redundancy may be built in to the nucleic acid code to ensure data integrity and quality. Examples of algorithms for encoding digital data to nucleic acid code are known in the art, see for example from Erlich et al. Science 355, 950-954 or Welzel et al., Nat. Comm. 14, 628

The nucleotides used for the nucleic acid-encoded data may preferably comprise the four canonical DNA bases adenine (A), thymine (T), cytosine (C) and guanine (G). Hence, the nucleic acid sequence preferably is a DNA sequence. It may be contemplated to use other types of nucleotides for nucleic acid, as long as the nucleotides used are compatible with the means and methods as described herein, i.e. PCR amplification and the like, such nucleotides can be contemplated. It may be envisioned to use an RNA molecule, wherein in the code uracil (U) is used instead of thymine. Hence, a nucleic acid using such an annotation may represent an RNA sequence. As data storage is of importance, and durable storage desirable, RNA may be less preferred. However, there can be situations wherein having less durable storage can be advantageous or preferred. It may be contemplated to have a nucleic acid code to comprises modified nucleotide bases such as, but not limited to 2′-fluoro- and 2′-O-methyl-dNTPs, 5-Methylcytosine (m5C), 5-Hydroxymethylcytosine (hm5C), N6-Methyladenine (m6A), N7-Methylguanine (m7G) or Inosine (I). It may be further contemplated to implement modified oligonucleotides such as phosphorthioate-containing oligonucleotides, also referred to as S-oligos, locked nucleic acids and variants thereof. Phosphorthioates are analogs of naturally occurring phosphodiester in which one oxygen is replaced by a sulphur. These type of modifications are known to increase DNA stability and hence may be advantageously used to prevent nucleolytic degradation. Hence nucleic acid modifications that advantageously may improve long term storage, (repeated) PCR amplification and/or sequencing of polynucleotide fragment, comprising such phosphorthioates and the like, may be contemplated in accordance with the invention.

Such modifications may be helpful to increase stability, while still retaining compatibility with the means and methods as described herein. The person skilled in the art is aware of the various types of natural and non-natural nucleotides and may use them accordingly when designing a nucleic acid-encoded dataset and may use appropriate annotations therefore.

The nucleic acid-encoded dataset of the method comprises a plurality of polynucleotide sequences. The polynucleotide sequences that comprise a single digital dataset are a plurality of different polynucleotide sequences that may have partially overlapping or identical regions therein. Overlapping polynucleotide sequences may be present in various ways such as, but not limited to, tandem overlap, convergent overlap, divergent overlap and/or in-phase or out-of-phase overlaps. In any case, when the plurality of polynucleotide sequences are combined, the polynucleotide sequences are to encompass the digital dataset that is provided.

The plurality of polynucleotide sequences each individually preferably have a length of between 50-500 nucleotides, preferably 100-250 nucleotides, more preferably 150-200 nucleotides. The inventors have advantageously found that polynucleotide sequences with a length of between 50-500 nucleotides can efficiently be localized into proteinosomes as described below (see also the examples herein). Without being bound by theory larger lengths than 500 nucleotides may be contemplated. For example, the plurality of polynucleotide sequences may comprise lengths in the range of between 50-1000, 50-2000 or even larger, which can be advantageous as the total number of individual polynucleotide sequences comprising a digital dataset can be reduced, and in alternative embodiments it may not be necessarily required to design polynucleotide sequences that need to localize in proteinosomes (see i.a. example 2 herein, and the like).

The polynucleotide sequences further comprise a forward and reverse primer binding site at the ends of the polynucleotide sequences to allow for PCR amplification. As said, the polynucleotide sequences at this stage are digital information and represent information of a single strand to allow for the preparation and/or synthesis of a double stranded polynucleotide fragment. It is to be understood that the forward and reverse primer binding sites are located such that forward and reverse primers, once double stranded polynucleotide fragments are made/provided, can bind with both strands of the double stranded polynucleotides and allow for PCR amplification of the sequences located between the primer binding sites. Primers, and corresponding primer binding sites, may typically be e.g. between 18 and 24 nucleotides in length, but longer or shorter primers may be contemplated. Hence, primers according to the invention are oligonucleotides, which are well known in the art, and any suitable primer pair can be easily made and generated or tested, or can be selected from known primer pairs in the art based on which forward and reverse primer binding sites can be incorporated at the ends of the polynucleotide sequences.

The polynucleotide sequences further comprise a locker target sequence wherein up to 100% of the individual polynucleotide sequences comprise at least one locker target sequence overlapping with a primer binding site. It is understood that not all of the individual polynucleotide sequences need to comprise a locker target sequence, for example only 1, 2, 3, 4, 5, 6, or more polynucleotide sequences of the plurality of polynucleotide sequences may comprise a locker. In another example, only a percentage of the polynucleotide sequences of the plurality of polynucleotide sequences comprise a locker, for example only 1%, 2%, 5%, 10%, 25%, 50%, 75% or more polynucleotide sequences of the plurality of polynucleotide sequences comprise a locker (e.g. Example 3 describes the use a locker in 6 out of 42 polynucleotide sequences) It is further understood that only one locker target nucleotide sequence in an individual polynucleotide sequence may have PCR amplification blocked with a locker of the corresponding double stranded polynucleotide fragment, though linear amplification of one of the strands of the double stranded polynucleotide fragment can still be linearly amplified. Hence, it may be preferred to have two locker target sequences in a polynucleotide sequence. As long as one polynucleotide sequence of the plurality of polynucleotide sequences comprises a locker target sequence, physical encryption can be achieved with a locker because the corresponding one double stranded polynucleotide fragment is not PCR amplified and the nucleic acid encoded dataset may not be completed in accordance with the invention. Without being bound by theory, it is understood that it can be difficult, to attain complete prevention of PCR-amplification (i.e. exponential amplification) of (double stranded) polynucleotide fragments comprising a locker target sequence by a locker oligonucleotide. The methods of the invention utilizing PCR amplification rely on binding of primers and/or lockers to polynucleotide fragments, and it may not be e.g. that in 100% of all instances it is prevented that a primer binds with its primer binding site even in the presence of locker target sequence and the locker. Alternatively, the locker in spite of being modified to block extension, said modification may not be 100% and/or DNA polymerase may not in 100% of the cases be blocked for extension, thus some amplification may occur from a locker (as if a primer). Nevertheless, PCR-amplification is severely reduced allowing for a highly substantial reduction of PCR-amplification as compared with the scenario without locker being presence. Hence, locking by a locker is understood to be absolute, but the amounts of amplified products is reduced to such an extend to prevent subsequent sequencing therewith not allowing to reconstitute all sequences, i.e. the complete data set is not obtained.

The reduction or blocking of PCR-amplification, as shown in the examples herein, can be assessed by means of measuring Ct (cycle threshold) values obtained with quantitative PCR reactions (qPCR) carried out with double stranded polynucleotide fragments that have been subjected to amplification (with and without unlocking, or with and without locker being present). (such qPCR utilizes primer binding sites and primers specific for a fragment, which are not to be mistaken for the (universal) primers used in the methods of the invention as outlined above). The polynucleotide fragments comprising a locker target sequence within a locked or unlocked system minus the Ct value as measured for polynucleotide fragments lacking a locker target sequence, is defined as ΔCt or delta-Ct. The ΔCt for the locked system, preferably is at least 1, 2, 3, 4, 5, 6, 7 or more. Having a higher ΔCt, indicates less relative amplification of locked fragments. The ΔCt for the unlocked system, preferably is close to 0. Having a low ΔCt in this scenario, indicates that all strands will be equally amplified when unlocked. Hence, the skilled is well capable of selecting appropriate lockers, primers, locker target sequences and primer binding sites that result in ΔCt values that are useful in accordance with the invention.

Hence, the plurality of polynucleotide sequences according to the invention comprises primer binding sites, and at least one of the plurality of polynucleotide sequences comprises a locker target sequence. This way, a nucleic-acid encoded data set is provided with which physical molecular encrypted nucleic-acid encoded data can be generated.

The locker target sequence is to provide for a binding site for a so-called locker, which locker, when bound with the double stranded polynucleotide fragment, is to block PCR amplification. The locker, similar to a primer, is an oligonucleotide. Hence, the locker target sequence is similar to a conventional primer binding site in that both are designed to allow the binding of an oligonucleotide (i.e. primer or locker). The locker, by binding to the locker target sequence, is to substantially prevent PCR amplification by the primers. The skilled person understands how to design, or confirm, that a locker can bind to the (complementary) locker target sequence. Experimental procedures to test whether a locker according to the invention can substantially block amplification can be such as described i.a. in example 1.1 and the skilled person is likewise well capable to do so when designing/selecting any further primer binding sites and locker target sequences and corresponding primers and locker.

When designing a suitable locker target sequence in accordance with the invention to which a locker can bind to outcompete the primer during PCR amplification, the primer binding site and locker target site may share a partially or completely, overlapping region of nucleotides within (substantially) the same polynucleotide sequence. Outcompeting according to the invention, means that one oligonucleotide preferentially binds to a sequence over another, or that one oligonucleotide can displace another already bound oligonucleotide. This can be accomplished by providing an oligonucleotide that has a higher affinity compared to the oligonucleotide it is to competes with, to a particular region. Multiple factors influence the binding interaction between an oligonucleotide and its binding site. Such as, but not limited to, sequence complementarity, melting temperature (Tm), length of the polynucleotide, GC content, and experimental conditions such as salt concentration, the presence of additives, temperature and buffer composition. For example, a suitable locker may have a Tm which is higher than the Tm of the primer. During PCR amplification after the denaturation stage, during the annealing stage, the locker will preferentially anneal with the locker target sequence therewith preventing, i.e. outcompeting, binding of the primer. Outcompeting can be obtained with a high concentration of locker. It may also anneal with the locker target sequence before the primer will anneal to the primer binding site, due to e.g. a high affinity of the locker and/or differential Tm. The locker may also dissociate a primer annealed with a primer binding site and displace the primer. Hence the locker is designed to outcompete the primer, and the skilled person is well capable of making such designs and testing thereof.

In step c) of the invention the polynucleotide sequences are synthesized to provide for polynucleotide fragments. The encoding and design of the plurality of polynucleotide sequences was performed in-silico in step b). In step c) the polynucleotide sequence information is used to provide for their corresponding physical entities, i.e. polynucleotide fragments, using a synthesizer.

The synthesis of polynucleotide fragments may be performed by a third party provider and many providers commonly provide such services. It may be opted to have polynucleotides synthesized by different providers in order to advantageously prevent a single provider from having all the information of a single dataset. This is advantageous as it prevents a single un-authorized party to access the information of a full nucleic acid-encoded dataset from a single provider. The synthesized fragments may be DNA or RNA polynucleotide fragments.

The polynucleotide fragments can be synthesized first as single stranded polynucleotide fragments and subsequently made into double stranded polynucleotide fragments or synthesized as double stranded polynucleotide fragments. The process of synthesizing may be such that a single strand is synthesized first and e.g. by means of a polymerase and primer made double stranded to form a double stranded polynucleotide fragment. The synthesizing may also be such that two complementary strands are synthesized, e.g. the plus- and minus-strand which are subsequently annealed to form a double stranded polynucleotide fragment. In either case, the information that is provided for in step b) is used in step c) for the preparation of, i.e. synthesis of double stranded polynucleotide fragments.

Step d) of the method comprises labelling of the double stranded fragments with biotin to therewith provide for biotin-labeled double stranded polynucleotide fragments representing the nucleic acid-encoded dataset.

The double stranded fragments comprise a biotin label on at least one of the two strands. Biotin labels are attached by biotinylation which covalently attaches biotin to a protein, nucleic acid or other molecule. The biotin label ensures that the fragments can bind to a biotin-binding protein and are used in subsequent steps for localizing the fragments in a compartment, such as a proteinosome. In a system where only a single strand (e.g. of the two) comprises a biotin label, the complementary strand (lacking a biotin label) may be localized by complementary base pairing with the strand that comprises the biotin label.

In a further embodiment, the method according to the invention comprises next the following step:
e) providing a locker complex comprising a locker and a locker anchor, wherein the locker is an oligonucleotide, wherein the locker comprises a sequence complementary to the locker target sequence, wherein the locker anchor is an oligonucleotide comprising a biotin label, wherein the locker and locker anchor have sequences complementary with each other, wherein the locker of the locker complex comprises a modification that blocks amplification, optionally wherein the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide, preferably wherein the locker and locker anchor complementarity is between 1-30, preferably 5-25, more preferably 10-20, most preferably 12-16 nucleotides in length;
to therewith provide for biotin-labeled double stranded polynucleotide fragments representing the nucleic acid-encoded dataset and locker complex, which are preferably mixed, wherein PCR amplification of the biotin-labeled double stranded polynucleotide fragments is repressed in the biotin-labeled double stranded polynucleotide fragments comprising the locker target sequence by preferential binding of the locker.

Although this step is denoted as e) herein, this does not necessarily mean that this step is to occur after step d). The e) indication here is simply to indicate the relationship to the preparation of the (biotinylated) double stranded polynucleotide fragments, which comprise locker target sequence(s), and the locker complex. This step can be performed simultaneously with the preparation of the fragments, or much later, and can also be performed at a different location. The preparation of a locker complex in this step is explained in more detail below.

Once the nucleic acid encoded data, i.e. plurality of polynucleotide sequences is provided or e.g. corresponding double stranded fragments thereof, the locker complex may be provided, the locker complex is designed such that is compatible with with the biotin-labeled double stranded polynucleotide fragments representing the nucleic acid-encoded dataset which comprise the locker target sequences.

The locker complex comprises a locker and locker anchor. The locker is an oligonucleotide that binds to the locker target sequence of a single stranded polynucleotide fragment. Binding of the locker to the locker target sequence prevents PCR amplification by outcompeting the primer and/or blocking of amplification. This is highly advantageous as (substantial) prevention of PCR amplification of these fragments can block unauthorized access to the complete data set comprised in the polynucleotide fragments. Therefore, the presence of the locker is to provide a physical barrier that interferes with amplification of polynucleotide fragments comprising the locker target sequence.

During a subsequent PCR amplification cycle, double stranded polynucleotide fragments like provided in step c) and d) first denature into two single stranded polynucleotide fragments. During the subsequent annealing step, the locker (preferentially) binds to the locker target sequence comprised in a single stranded polynucleotide fragment. The primer (preferentially) can bind with denatured polynucleotide strands if lacking a locker target sequence. However, denatured polynucleotide strands comprising a locker target sequence are preferentially bound by the locker. Hence the primer is substantially outcompeted.

The locker anchor is also an oligonucleotide and when bound with the locker it forms the locker complex. The locker anchor further comprises a biotin label similar to the biotin labels as described for the polynucleotide fragments in step d). By binding of the locker to the locker complex and interaction of the biotin label to a biotin-binding agent, e.g. a protein, the locker complex as a whole can be localized in a proteinosome in a subsequent step. The locker anchor is designed such that it does not substantially interferes with the PCR, i.e. does not interfere with the process of allowing the locker to block PCR amplification as described above. The locker anchor serving to retain the locker into proteinosomes via complementary base pairing between locker anchor and locker and via binding of the biotin of the locker anchor with the biotin-binding agent within the proteinosome.

In addition, in a further embodiment, the method according to the invention comprises the following steps:
f) providing a biotin-binding protein, comprising one or more biotin binding domains, wherein a plurality of the biotin-binding protein is provided localized in a plurality of proteinosome compartments;
g) localizing the biotin-labelled double stranded polynucleotide fragments and the locker complex in the proteinosome compartments, optionally wherein 50-100%, preferably 75-100%, more preferably 90-100% of the biotin-labelled double stranded polynucleotide fragments representing the nucleic acid encoded dataset are localized in a single proteinosome compartment and wherein the biotin-labelled double stranded polynucleotide fragments cover the nucleic acid encoded dataset least once;
h) storing the proteinosome compartments containing the localized biotin-labelled double stranded polynucleotide fragments, locker complex and biotin-binding protein, to therewith provide for physically molecularly encrypted nucleic acid-encoded data.

These steps are as explained in more detail below.

Step f) of the method according to the invention further comprises providing a biotin-binding protein, which comprises one or more biotin binding domains, wherein a plurality of the biotin-binding protein is provided localized in proteinosome compartments.

Biotin-binding agents, e.g. a biotin-binding proteins, which are well known in the art and as described in the examples herein, according to the invention bind biotin, i.e. biotin such as used in labeling the polynucleotide fragments or locker anchor as described herein. The biotin-binding agent used in accordance with the invention is resistant to the temperatures used in the PCR amplification, i.e. it is to retain its biotin binding function when subjected to high temperatures. Hence, biotin-binding agents are for example the tetrameric biotin-binding protein such as, but not limited to the thermostable biotin-binding protein Tamavidin 2-HOT, and derivatives thereof.

Proteinosomes, according to this invention, are semipermeable compartments based on, but not limited to, protein-polymer conjugates e.g. prepared by covalently crosslinking bovine serum albumin (BSA) and poly(N-isopropylacrylamide) (PNIPAm) (see Methods). As described in the examples herein, advantageously BSA was used as it is commonly available and cost-effective.

The proteinosome compartments in accordance with the invention are temperature-controlled meaning that the permeability of the proteinosomes is dependent on the temperature. That is, the proteinosomes according to the invention are permeable i.e. have space between the protein-polymer conjugates, i.e. the proteinosomes are ‘open’ which allows for diffusion of e.g. oligonucleotides (not bound by a biotin-binding agent), in and out of the proteinosome, which permeability depends on temperature. At elevated temperatures, the permeability decreases due to a collapse of the protein-polymer conjugates. This prevents oligonucleotides (and (double stranded) polynucleotides) to diffuse out of the proteinosome, i.e. the proteinosomes are ‘closed’. During PCR amplification, which typically is performed at elevated temperatures up to 95 degrees Centigrade, double stranded oligo- and polynucleotides denature into single stranded oligo- and polynucleotides. Because the proteinosomes are closed at elevated temperatures denatured oligo- and/or polynucleotides that are not bound to a biotin-binding agent do not diffuse out of the proteinosome. When the temperature decreases, below the elevated temperatures typically used for PCR, the proteinosome become permeable again and the single stranded oligo- and polynucleotides can substantially anneal back with their complementary single stranded oligo- or polynucleotides, and if these comprise a biotin label, these will be retained in the proteinosome if comprising a biotin-binding agent. Double stranded oligo- or polynucleotides without a biotin label, may diffuse out of the (now) permeable proteinosome and are not retained.

Localization in accordance with this invention means that a component is inside a compartment. The biotin-binding protein according to the invention are localized inside the proteinosome compartment and can freely move within the confounds thereof. In accordance with the invention, the biotin-binding protein will not diffuse out due to the available space between the protein-polymer conjugates, nor are actively transported out of the proteinosome compartment. Hence, the proteinosome compartment is formed around multiple biotin-binding proteins (see Methods). This is advantageous because any biotin-labelled component, such as the locker anchor or polynucleotide fragments described above, will interact with the biotin-binding protein and is subsequently also localized inside a proteinosome compartment. The inventors advantageously make use of biotin-labelled components which can freely move into the proteinosome compartment and remain localized there as long as the interaction between the biotin and biotin-binding protein is established.

Step g) of the method according to the invention involves localizing the biotin-labeled double stranded polynucleotide fragments and the locker complex in the proteinosome compartments such as shown e.g. in the examples herein (see ‘Localizing DNA in proteinosomes’). Briefly, biotin-labeled double stranded polynucleotide fragments are put in a suspension comprising the previously prepared proteinosomes. The fragments can diffuse into proteinosomes and the biotin label of the fragments will interact with the biotin-binding protein thereby localizing the fragments in the proteinosomes. The same procedure is performed, before, simultaneously or subsequently, for the locker complex. Localizing the locker complex in the proteinosome is based on the same technical premise as localizing the biotin-labeled double stranded polynucleotide fragments.

Preferably, all unique biotin-labeled double stranded polynucleotide fragments representing the nucleic acid-encoded dataset are distributed across the plurality of proteinosome compartments at least once. How and to what extent the double stranded polynucleotide fragments are distributed over the proteinosomes is defined by chance during the process of localizing. It can be assumed that each proteinosome comprises a randomly distributed sample of the plurality of polynucleotide fragments. It may be preferred that a single proteinosome, on average, comprises more than one of each of the unique double stranded polynucleotide fragments. In any case, the complete dataset comprised in the plurality of polynucleotide fragments is distributed over the plurality of proteinosome compartments. It is understood that the number of biotin-labeled double stranded polynucleotide fragments, per unique double stranded polynucleotide sequence, is to be represented at least once, and preferably at least about 10 times, these to be distributed over the plurality of proteinosomes. It is further understood that, depending on the file size of the digital dataset as provided in step a) and the length of the polynucleotide sequences as encoded in step b), the amount of total polynucleotide fragments in step c) may vary. The amount may vary even when starting with the same digital dataset in the case encoding is performed different in step b) according to the invention. It is further understood that depending on the amount of total polynucleotide fragments, the total amount of proteinosomes required may vary and this may further vary depending on e.g. the size and/or size distribution of the proteinosomes.

The number of proteinosome compartments and/or number of fragments for each unique polynucleotide sequence needed to achieve sufficient coverage for subsequent sequencing may vary as this may depend i.a. on the number of unique polynucleotide sequences, and may be tested and confirmed by taking a sample of prepared and localized proteinosome compartments and confirming (e.g. by PCR amplification and sequencing) that the original digital dataset can be recovered. Hence, the skilled person is well capable of selecting suitable conditions for (amplified) polynucleotide fragment copy number and distribution across proteinosomes.

### Step h) Storing the Proteinosome Compartments

Step h) of the method according to the invention involves storing of the proteinosome compartments containing the localized biotin-labeled double stranded polynucleotide fragments, locker complex and biotin-binding protein, to therewith provide for physically molecularly encrypted nucleic acid-encoded data.

Preferably, the stored compartments and hence the components therein are protected from water and light and are stored at low temperatures, such as below zero degrees Centigrade. Proteinosome compartments comprising the components can be lyophilized, e.g. in the presence of trehalose (a reducing sugar known to enhance DNA stability). Such storing is advantageous because it allows durable storage of the proteinosomes and hence protects the data for long-term use. Without being bound by theory, lyophilized proteinosomes can be stored for decades or more such as centuries to millennia. Lyophilization may be performed using a lyophilizer and the skilled person understands how to select the proper conditions with which a sample may be lyophilized. Storage may be done in e.g. a cooler or freezer for storing at 0, −20, −40 of −80 degrees Centigrade. Such freezers are commonly available.

In one embodiment, steps f-h) are performed separated from of steps a)-d) and/or step e) as described above. This can be separated in time and/or physically e.g. different locations or by different persons. For example, the biotin-labeled double stranded polynucleotide fragments of step d) and the locker complex of step e) are provided by two different persons, a third person combines these components with the components of step f) and performs subsequent steps g-h). This is highly advantageous as up to this point, no single person has all the information to recapitulate the system as a whole, hence the security of the digital data can be further enhanced.

In a further embodiment, the method according to the invention comprises the following step:
i) providing proteinosome compartments as obtained in step h);
j) providing an unlocker, wherein the unlocker comprises an oligonucleotide sequence complementary to the locker of the locker complex, wherein the complementarity between the unlocker and locker is longer than the complementarity between the locker anchor and locker;
k) mixing the unlocker and the proteinosome compartments in a suspension allowing the unlocker to interact with the locker of the locker complex thereby forming a double stranded complex of the locker and the unlocker, resulting in substantially displacing the double stranded complex of the locker and the unlocker out of the proteinosome compartment by toehold mediated strand displacement, preferably washing the mixed suspension, thereby removing, by diffusion, the double stranded complex of the locker and the unlocker from the proteinosome compartments, preferably wherein the interaction between the locker strand and the unlocker is performed between 0-32 degrees Centigrade, preferably between 5-30 degrees Centigrade, more preferably between 15-25 degrees Centigrade,
therewith obtaining proteinosome compartments comprising biotin-labeled double stranded polynucleotide fragments representing the nucleic acid-encoded dataset and the locker anchor, from which the locker has been substantially removed;
l) amplifying the biotin-labeled double stranded polynucleotide fragments to therewith obtain amplified unlocked polynucleotide fragments, preferably using a forward universal primer and a reverse universal primer targeting the forward and reverse primer binding sites.
m) optionally wherein the amplified unlocked polynucleotide fragments and the proteinosome compartments comprising biotin-labeled double stranded polynucleotide fragments representing the nucleic acid-encoded dataset and the locker anchor are separated from each other, and wherein to the proteinosome compartments comprising biotin-labeled double stranded polynucleotide fragments and the locker anchor, a locker is localized to therewith reconstitute the locker complex localized in the proteinosome compartments, and subsequently storing the reconstituted proteinosome compartments with biotin-labeled double stranded polynucleotide fragments and locker complex.

The above steps are as explained in more detail below.
In step i) proteinosome compartments as obtained in step h) are provided. The proteinosome compartments comprise at least a locker complex comprising a locker and biotin-labeled locker anchor, a plurality of biotin-labeled double stranded polynucleotide fragments and a biotin-binding agent. The proteinosome compartments may be retrieved from storage (such as a freezer) and may need thawing, and/or resuspension in a suitable buffer. It is understood that when retrieving the proteinosomes from storage and providing in step i) they are provided in a suitable buffer allowing for the subsequent steps to be performed.

In step j) an unlocker is provided. The unlocker comprises an oligonucleotide sequence complementary to the locker of the locker complex, wherein the complementarity between the unlocker and locker is longer than the complementarity between the locker anchor and locker. The complementarity between the unlocker and locker is preferably 1, 2, 3, 4, 5 or more nucleotides longer than the complementarity between the locker anchor and locker. The complementarity between the unlocker and locker may be the full length of the unlocker, i.e. all nucleotides of the unlocker interact with (part of) the nucleotides of the locker, which necessarily means that the locker anchor is not fully complementary with the locker.

Hence, in accordance with the invention, the complementarity and thus the number of nucleotides that base pair between the unlocker and locker is more than the number of nucleotides that base pair between the locker anchor and locker. This enables toehold mediated strand displacement. The driving force behind the toehold mediated strand displacement is the decrease in free energy that comes from an increase in the number of paired bases.

In another aspect of the invention, the unlocker as described herein, as used in e.g. step j), advantageously comprises a modification to block amplification. Blocking amplification in this aspect refers to blocking the unlocker to function as primer itself, as it is a short polynucleotide, like a primer. Highly preferably, such a modification does not allow extension of the unlocker sequence by a polymerase when (by happenstance) base paired with a polynucleotide, such as a polynucleotide fragment that is to comprise/encode data to which access can be locked and unlocked in accordance with the invention. Of course, the unlocker may be designed to avoid such undesired base pairing, but such may occur to some extent, and, by the modification of the unlocker sequence according to this embodiment may avoid unintended amplification of an unlocker bound base paired with a polynucleotide. Such a modification can be the same as used for the locker described above. Hence, in a further embodiment, in accordance with the invention, the unlocker comprises a modification selected from comprising a 3′ inverted dT nucleotide a 3′ dideoxycytidine (ddC), a C3 phosphoramidite spacer, a 3′ amino modifier and a 3′ phosphorylation. Preferably the modification is an 3′ inverted dT nucleotide. Such an unlocker in accordance with this embodiment of the invention that blocks unintended amplification of a polynucleotide (i.e. does not allow extension of the unlocker if bound with a polynucleotide), is in particular advantageous when a data file is to be repeatedly accessed (see e.g. Example 5), i.e. is to be accessed more than once. In case a data file has been unlocked once, some residual unlocker may remain which in itself may serve in a subsequent attempt at accessing data, as a primer. By having an unlocker comprising a modification that blocks amplification, the risk of unwanted or unauthorized access (i.e. without an unlocker) in the locked state can be further reduced.

Accordingly, in step k) the unlocker and the proteinosome compartments are mixed in a suspension allowing the unlocker to enter into the proteinosome and interact with the locker comprised in the locker complex thereby forming a double stranded complex of the locker and the unlocker, resulting in substantially displacing the locker anchor. Such a process is known as toehold mediated strand displacement. The result in step k) thereof is that in the proteinosome a complex of locker and unlocker is formed, which does not have a biotin-label, which locker/unlocker complex can subsequently diffuse out of the proteinosome compartment, while the locker anchor remains. This way, the locker can be substantially removed from the proteinosome compartments, e.g. when such steps are repeated one or more times.

A proteinosome according to the invention is a compartment and the terms may be used together, separately or interchangeably and refer to the same entity unless the context specifies otherwise, i.e. reference may be made to proteinosome, compartment, proteinosome compartment, which terms in the context of proteinosomes is understood to refer to the same entity. In one preferred embodiment, the proteinosome compartment is a crosslinked polymer-protein hybrid of bovine serum albumin and poly(N-isopropylacrylamide).

Toehold-mediated strand displacement involves the displacement of a pre-bound strand (i.e. locker/locker anchor in a complex) by a complementary invading strand (i.e. unlocker), initiated by the recognition of a short toehold sequence, enabling dynamic control in DNA and RNA strand exchange processes. The process of toehold-mediates strand displacement is well known in the art. Toehold mediated-strand displacement may occur at room temperature and no elevated temperatures as used in e.g. PCR amplification are required. By mixing the locker and the proteinosome compartments, the locker diffuses over the protein-polymer conjugate membrane and into said proteinosome. Once within the proteinosome the unlocker interacts with the locker. It is understood that, when mixing the unlocker and proteinosome compartments that are temperature controlled, the temperature is such that it allows the diffusion of the locker into the proteinosome compartment.

The process of removal of the double stranded complex of the locker and the unlocker is also diffusion driven. Given a sufficient volume of the suspension, a substantial amount of locker, complexed with unlocker, will be displaced from the compartments by diffusion over the proteinosome protein-polymer conjugate membrane.

Step k) according to the invention involves obtaining proteinosome compartments comprising biotin-labeled double stranded polynucleotide fragments representing the nucleic acid-encoded dataset and the locker anchor, from which the locker has been substantially removed. The inventors advantageously realized that by utilizing toehold mediated strand displacement, double stranded DNA, such as the biotin-labeled polynucleotide fragments in the proteinosomes remain intact during this process and do not denature. Consequently, the double stranded polynucleotide fragments are not affected and are retained, and only the locker is removed from the proteinosomes, thereby unlocking the polynucleotide fragments and allowing subsequent amplification and/or sequencing of polynucleotide fragments.

Step l) of the method according to the invention involves amplifying the biotin-labeled double stranded polynucleotide fragments to therewith obtain amplified unlocked polynucleotide fragments.

The amplified unlocked polynucleotide fragments according to the invention include those fragments that have been amplified and have not been blocked by the locker. The primers used for amplification may include the same primers as used in step c), or substantially the same primers. The same primer binding sites, or substantially the same primer binding sites as designed in the polynucleotide sequences as defined in step b) may be used. The primers used for amplification may thus be the same primers as the primers originally provided, e.g. when in step c) the preferred amplification is performed. However, the primers for amplifying in this step may be different and may comprise e.g. additional sequences, e.g. sequences that may be useful e.g. for subsequent capture and/or sequencing of amplified unlocked polynucleotide fragments. The amplified unlocked polynucleotide fragments thus represent both polynucleotide fragments comprising the locker target sequence and polynucleotide fragments lacking the locker target sequence. Thus, combined the amplified unlocked polynucleotide fragments represents the nucleic acid-encoded dataset, which in turn represents the digital dataset.

Preferably, amplification is performed using conventional PCR techniques. The skilled person understands how to use such techniques. Amplification of the fragments is attained using primers which bind to primer binding sites. In a preferred embodiment the amplification is with a forward universal primer and/or a reverse universal primer targeting the forward and/or reverse primer binding sites. These universal primers may be the same as the primers used in step c). The set of universal primers consists of at least a single pair of—forward and reverse—primers that bind to the forward and reverse binding primer sites of the plurality of polynucleotide fragments. It is understood that in accordance with the invention, a primer may be regarded to be a universal primer when it binds to substantially all polynucleotide fragments which are to have the same primer binding site(s) in a nucleic acid-encoded dataset. Of course, it may be contemplated to use one or more different combinations of primer pairs instead of a universal primer pair.

In one embodiment, step I) is performed separated from the other steps described above. This can be separated in time and/or physically e.g. different locations or by different persons. This is advantageous as the amplification thereof may be performed by a trusted provider of such services. Such a provider may also perform the subsequent sequencing of the amplified unlocked polynucleotides to provide for the sequences of the nucleic acid-encoded data comprising the digital dataset. The obtained sequences may be returned, directly or indirectly, to the authorized person who can decode the nucleic acid-encoded data to retrieve the original digital dataset.

In an optional step m) the amplified unlocked polynucleotide fragments and the proteinosome compartments comprising biotin-labeled double stranded polynucleotide fragments representing the nucleic acid-encoded dataset and the locker anchor are separated from each other and are subsequently isolated, and wherein to the proteinosome compartments comprising biotin-labeled double stranded polynucleotide fragments and the locker anchor, a newly-provided locker is localized to therewith reconstitute the locker complex that was localized in the proteinosome compartments, and subsequently storing the reconstituted proteinosome compartments with biotin-labeled double stranded polynucleotide fragments and locker complex. In this embodiment, the biotin-labeled polynucleotide fragments and locker anchor are separated from the amplified polynucleotide fragments. Hence proteinosome compartments are provided that comprise biotin labeled polynucleotide fragments and a locker anchor, without a locker. To re-lock the unlocked polynucleotides, a locker can be newly reintroduced. The locker interacts with the biotin-labeled locker anchor and a (new) locker complex is formed, i.e. reconstituted. The proteinosome comprising the re-locked polynucleotide fragments can next be processed and stored for later use. This way, advantageously, the amount and/or number of data storage units (e.g. number of separate containers, or, number of biotin-labeled double stranded polynucleotide fragments), may be advantageously retained. The re-locked polynucleotide fragments can be unlocked again, i.e. for the second or a further time, in accordance with any of the previous step(s) performed for unlocking the polynucleotides for the first time. Advantageously, the inventors have shown (i.a. in Example 3) that this process can be performed multiple times, and that each time the nucleic acid-encoded data is effectively re-locked and cannot be decoded when in the (re-) locked state, but is effectively unlocked such that the data stored on the polynucleotide fragments can be retrieved, i.e. decoded.

In one embodiment according to the invention, the method for providing a physical molecular encryption of nucleic acid-encoded data system comprises the steps of:
a) providing a digital dataset;
b) encoding the digital dataset to a nucleic acid code, thereby designing a nucleic acid-encoded dataset, wherein the nucleic acid-encoded dataset comprises a plurality of polynucleotide sequences of, preferably, between 50-500 nucleotides, wherein each of the polynucleotide sequences further comprise a forward and reverse primer binding site at the ends of the polynucleotide sequences that allows for subsequent PCR amplification and wherein up to 100% of the individual polynucleotide sequences comprise at least one locker target sequence wherein the locker target sequence is located such that PCR amplification is blocked when a locker is bound to the locker target sequence;
c) synthesizing the designed plurality of polynucleotide sequences to provide for polynucleotide fragments, wherein the polynucleotide fragments are synthesized as single stranded polynucleotide fragments and subsequently made double stranded polynucleotide fragments or synthesized as double stranded polynucleotide fragments;
d) labeling the double stranded polynucleotide fragments with biotin to therewith provide for biotin-labeled double stranded polynucleotide fragments representing the nucleic acid-encoded dataset.
e) providing a locker complex comprising a locker and a locker anchor, wherein the locker is an oligonucleotide, wherein the locker comprises a sequence complementary to the locker target sequence, wherein the locker anchor is an oligonucleotide comprising a biotin labeled anchoring sequence, wherein the locker and locker anchor have sequences complementary with each other, wherein the locker of the locker complex comprises a modification that blocks PCR amplification of polynucleotide fragments from the primer binding site;
to therewith provide for biotin-labeled double stranded polynucleotide fragments representing the nucleic acid-encoded dataset and locker complex, wherein PCR amplification of the biotin-labeled double stranded polynucleotide fragments is repressed by preferential binding of the locker to the locker target sequence comprised in the biotin-labeled double stranded polynucleotide fragments.
f) providing a biotin-binding protein, comprising one or more biotin binding domains, wherein a plurality of the biotin-binding protein is provided localized in a plurality of proteinosome compartments;
g) localizing the biotin-labeled double stranded polynucleotide fragments and the locker complex in the proteinosome compartments and wherein the biotin-labeled double stranded polynucleotide fragments are distributed over the proteinosome compartments thereby covering the nucleic acid-encoded dataset at least once;
h) storing the proteinosome compartments containing the localized biotin-labeled double stranded polynucleotide fragments, locker complex and biotin-binding protein;
therewith providing for the physically molecularly encrypted nucleic acid-encoded data.

In one embodiment, the primer binding site comprised in the polynucleotide sequence as designed in step b) has a different nucleotide feature upstream thereof, but is still located substantially towards the 5′ or 3′ end of the polynucleotide. The skilled person understands that suitable primer and corresponding suitable primer binding sites may have a minimum and/or maximum length in order to properly function as a primer binding site. It is well within the capabilities of a skilled person to select from the prior art, or newly design, suitable primer binding sites and primers. Suitable primers and hence corresponding primer binding sites may be selected or designed, generated and tested. It is routine for a skilled person in the art to confirm, either in-silico or experimentally, whether such a primer and/or primer binding site could be used for amplification of a sequence in the context of the present invention. The polynucleotide sequences of step b) according to the invention represent (in-silico) information, data, and not the physical molecule. It is understood that PCR amplification is to be performed with synthesized polynucleotides, i.e. the corresponding polynucleotide fragments.

In one embodiment, the locker target sequence is overlapping with one of the primer binding sites.

In one embodiment, the polynucleotide sequences comprise an overlap between the locker target sequence and primer binding site wherein said overlap is between 1-30, preferably 5-25, more preferably 10-20, most preferably 12-16 nucleotides in length.

In one embodiment a suitable locker target sequence to prevent PCR amplification is located 3′ of the primer binding site as present in the corresponding double stranded polynucleotide fragment, and the primer binding site and locker target sequence do not overlap. Both primer and locker oligonucleotides may bind to their corresponding binding sites but amplification is prevented once the polymerase (or otherwise) reaches the locker due to the presence of an amplification blocking feature in the locker. Without being bound by theory, having the locker bound 3′ of the primer binding site may hinder or reduce efficiency of polymerase extension. For example, polymerases that may not have display strand displacement activity can be blocked by the presence of a downstream bound locker when the polymerase is extended from the primer bound with the primer binding site and when subsequently reaching said downstream bound locker, the polymerase is not (or less) capable of displacing the locker, hence amplification is blocked. It may be contemplated to use such type of polymerases in accordance with the invention as it may be e.g. advantageous to select this functional feature. In case polymerases are used with displacement function, it is understood it is preferred that the target nucleotide sequence is not located 3′ of a primer binding site as present in the corresponding double stranded polynucleotide fragment.

Of course, in embodiments where the primer binding site and locker binding site do not overlap, the primer and locker may bind independently of one another and there is no competition for binding thereof. Subsequently, during extension, polymerase may bind to the single stranded fragment having a locker or primer bound. Highly advantageously, due to the presence of a modification comprised in the locker, a polymerase cannot extend therefrom when present. Hence, the single stranded fragment which has a locker bound to it may in such a scenario not be copied and therefore the double stranded polynucleotide fragment as a whole is not PCR amplified.

In one embodiment, the individual polynucleotide sequences may have more than one, such as two, three, four or five locker target sequences located 3′ from the primer binding site. Such a plurality of locker target sequences may for example allow improved prevention of amplification when using two or more locker. It may also allow for example for more than one entity to lock the polynucleotides with a personal locker.

In a further embodiment, the individual polynucleotide sequences have a at least two locker target sites. That is, at both ends of the polynucleotide sequence a primer binding site and locker target site is located. This way, linear amplification that can occur during a PCR reaction is also prevented/reduced because both of the complementary strands of the double stranded polynucleotide fragments will have a locker bound therewith to interfere with amplification by the primers. Hence, in this embodiment, both plus and minus strands of a double stranded polynucleotide fragment, will each have a locker target sequence with a locker bound therewith such that both linear and PCR-amplification can be prevented.

In one embodiment, the plurality of polynucleotide sequences all have the same locker target sequence in each of the polynucleotide sequences. Hence, the double stranded polynucleotide fragments all comprise the same locker target sequence. This has the advantage that only one unique locker is required to repress amplification.

In another embodiment, the locker target sequence is unique for each of the plurality of polynucleotide sequences. Hence, each double stranded polynucleotide fragment comprises a unique locker target sequence to which a plurality of lockers may bind.

In yet a further embodiment, a single locker can bind to a unique locker target sequences. Hence, each double stranded polynucleotide fragment comprises a unique locker target sequence to which only a corresponding single locker may bind.

These embodiments have the advantage that in principle one or more unique lockers, and hence one or more unique unlockers, are required to lock and unlock the polynucleotide sequences comprising a locker target sequence. It has the further advantage that the unique locker target sequence can be used for identifying polynucleotide sequences comprising said unique locker target sequence belonging to distinct digital datasets. Such polynucleotides with unique locker targets may not need an indexing sequence for the purpose of identifying to which digital dataset they belong.

In another embodiment, not all polynucleotide sequences have the same unique locker target sequence but there are at least two unique locker target sequences across the plurality of polynucleotide sequences. For example, polynucleotide sequences 1-10 have locker target sequence “LT1” and polynucleotide sequences 11-20 have locker target sequence “LT2”.

In accordance with the previous embodiments, it may be contemplated that more than one locker target sequence, of the plurality of locker target sequences, is present on a single polynucleotide sequence.

In one embodiment, encoding of the digital dataset involves the implementation of a primer binding site and a locker target site in the nucleic acid code. Hence, during encoding of the digital dataset to a nucleic acid-encoded dataset, a plurality of polynucleotide sequences is provided that does not need any further manual editing. Hence, the encoding can include the implementation of the primer binding sites and locker target sites. Such encoding advantageously allows a person to efficiently convert a digital dataset to a nucleic acid-encoded dataset without further manual intervention. Of course, this is not a requirement and can also be performed separately, as separating security measures can add further levels of security.

In one embodiment, the locker target sequence of the polynucleotide sequence is part of the nucleic acid-encoded dataset. This advantageously provides an increase in the information density across the plurality of polynucleotide sequences because the locker target sequence itself also comprises information on the digital dataset. In general, it is understood that in accordance with the invention, particularly in steps a) and b) thereof, to providing and encoding a digital dataset to a nucleic acid code allows for optimizing data density. Hence, in a further embodiment, the primer binding site of the polynucleotide sequence, is part of the nucleic acid-encoded dataset. This, similar to the previous optional embodiment, also has the advantage of improved data density.

In yet another embodiment the polynucleotide sequences comprise an indexing sequence. For example, indexing sequences may be used to determine the order of the plurality of polynucleotide sequences. In another example wherein more than one digital dataset is encoded to a nucleic acid-encoded dataset, indexing sequences may be used to distinguish between polynucleotides belonging to the same dataset, this can be in addition to providing information on the order of the plurality of polynucleotide sequences. These indexing sequences, similar to a locker target sequence and/or a primer binding site may be part of the nucleic acid-encoded dataset to improve data density.

In one embodiment a plurality of digital datasets is provided wherein each of the digital datasets is encoded, thereby designing a plurality of nucleic acid-encoded datasets. In a further embodiment the plurality of datasets each comprise a plurality of polynucleotide sequences comprising a unique indexing sequence for decoding, ordering of polynucleotide sequences and/or identifying the nucleic acid-encoded dataset to which the polynucleotide sequences belong. Of course, each of the plurality of datasets may each have unique primer binding sites for amplification and/or (a) unique locker target sequence(s), which allows for differentiation between different datasets. Highly advantageously, the inventors have shown that when two digital datasets were provided, wherein each of the two digital datasets was encoded to two individual nucleic acid codes, that each of the two individual nucleic acid-encoded datasets could be, individually, locked and unlocked as well as effectively be decoded such that the original datasets are retrieved. The inventors have found, and as exemplified in Example 4, that when one data file is unlocked (i.e. by providing an unlocker), the other dataset remained locked, and vice versa.

In an embodiment, it may be advantageous to use the locker, unlocker and/or locker target sequence according to Example 3 or Example 4, i.e. SEQ ID NO: 15, 21, 18 and/or 22 (Table 6).

In an embodiment 500, 1000, 5000, 10.000, 50.000 or more simulated decoding attempts are required before an amplified and sequenced dataset of a locked file can be decoded. To test whether the designed nucleic acid-encoded dataset according to the invention complies with such a threshold, the skilled person can for example test this according to the means and methods described in Example 3.

The skilled person understands that in accordance with the invention, in particular wherein a plurality of individual nucleic-acid encoded datasets is used, molecular encrypted (also referred to as locked) nucleic acid encoded datasets are designed in such a way that it would require a very high average coverage in order to be able to retrieve a particular locked nucleic acid-encoded dataset without any unlocking. The average coverage of a sequence relates to the number of unique sequences and the number of reads obtained for each of the unique sequences. The number of reads that may be obtained of a locked nucleic acid-encoded dataset as compared with an unlocked nucleic acid-encoded dataset is usually very low, in particular after amplification. The higher the average coverage required to obtain sufficient reads of sequences representing a locked an encrypted nucleic acid-encoded data set, the stronger the locked nucleic acid-encoded dataset is locked. It is understood that the higher the average coverage of sequencing, the higher the chance that a sequence or DNA molecule present at very low concentrations (e.g. sequences comprising a locker target sequence which are not amplified) and comprised in the amplified pool would be sufficiently sequenced. Thus, designing the encrypted nucleic acid-encoded dataset in such a way that it requires a high average sequencing coverage in order to lead retrieve the data is highly advantageous. The design is highly preferably such that under normal conditions (i.e. the conditions under which the datasystem is designed to operate) when the system is used, the average coverage required to obtain sufficient reads representing a locked dataset is much too high therefor. For example, as shown in examples, at an average coverage of 30, for a particular locked file, statistically in 99.2% of the times, the locked file was not accessed. Hence, the higher the coverage required to have a locked dataset statistically remained locked in e.g. at least 99% of the times, the stronger the molecular encryption. The skilled person is able to test and confirm means and methods in accordance with the invention such that under the conditions the system is to operate, it allows for sufficiently secure locking/encryption of selected datasets. The average coverage required to obtain sufficient reads in a defined percentage of attempts to read a file (e.g. 99%), of a particular uncrypted dataset, e.g. when comprised in a plurality of individual nucleic-acid encoded datasets, is representative of the quality of molecular encryption/locking. The higher the average coverage required, the higher the quality of molecular encryption/locking. Conversely, the percentage of attempts to read a locked file at a defined coverage (e.g. 30), is representative of the quality of molecular encryption/locking. The higher the coverage and/or % of attempts required, the better the quality of molecular encryption/locking. The fold increase in average coverage required for decoding a locked file compared to an unlocked file may also be used as a quality parameter for the molecular encryption/locking. Without being bound by any particular theory or practical example, successful decoding of an unlocked file in accordance with the invention may require an average coverage of 15. Successful decoding of an locked file in accordance with the invention may require an average coverage of 1500, hence decoding the unlocked file requires a 100-fold increase in average coverage. The higher the fold increase, the stronger the encryption/locking

In one embodiment an indexing sequence is comprised in the polynucleotide sequences, which may be used for decoding, ordering of polynucleotide sequences and/or identifying the nucleic acid-encoded dataset to which the polynucleotide sequences belong. The indexing sequence may also be referred to as a barcoding sequence. It can be used to determine inter- and intra-sequence relations, i.e. the order of sequences within a dataset and distinguishing sequences between datasets. Hence, when the indexing sequence is used for, but not limited to, ordering of the sequences, the indexing sequence has a unique identifier which allows for indicating the order. In one embodiment the indexing sequence is used in decoding, wherein the indexing sequence indicates which specific rules and procedures should be used to decode the sequence. It is advantageous to implement indexing sequences as it can improve decoding, ordering or identification efficiency when working with large or multiple digital datasets.

In one embodiment the indexing sequence comprises a unique identifier for identifying the nucleic acid-encoded dataset to which the polynucleotide sequences belong.

In one embodiment according to the invention, the nucleic acid-encoded dataset is a DNA encoded dataset, and the biotin-labeled double stranded polynucleotide fragments are double stranded DNA fragments. It may be highly preferred to use standard DNA nucleotides, A, C, G, and T, such as typically used in synthesis of polynucleotides, and such as typically used in standard PCR reactions, as such may be advantageous from a cost perspective while allowing to provide for double stranded polynucleotides with good and durable stability. Of course, as said, it may be contemplated to include modified nucleotides, or even use RNA nucleotides, or the like, as long as double stranded polynucleotide fragments can be provided that are compatible with the means and methods as described herein.

In one embodiment the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide. The preferred modification is an inverted dT nucleotide which is known in the art to prevent amplification. The 3′ inverted dT can be incorporated at the 3′-end of an oligonucleotide, leading to a 3′-3′ linkage which inhibits both degradation by 3′ exonucleases and extension by DNA polymerases. A further advantage of using inverted dT in the locker is that such modified nucleotides can still base pair and thus contribute positively to the degree of complementarity between two base pairing strands.

In one embodiment, the locker and locker anchor complementarity is between 1-30, preferably 5-25, more preferably 10-20, most preferably 12-16 nucleotides in length. In the method according to the invention, it is preferred that the locker anchor oligonucleotide is as short as possible whilst maintaining the ability to form sufficient complementarity with the locker to keep the locker complex localized in the proteinosome when a locker complex is formed. In a preferred embodiment the complementarity between the locker and locker anchor is such that the locker complex remains intact, i.e. remains double stranded and does not denature, at temperatures up to 50 degrees centigrade. As said, during the PCR amplification step, the locker anchor is to not interfere with the locker function and is dissociated from the locker. Hence, the skilled person knows how to design or choose a nucleotide sequences to comply with these criteria.

In a further embodiment, the locker anchor and locker are not of the same nucleotide length. For example, the locker anchor may be 15 nucleotides in length, whereas the locker may be 43 nucleotides in length. In such an embodiment, the complementarity between the locker anchor and locker may be less than the number of nucleotides of the locker anchor itself. For example, the locker anchor can be 15, and 14 nucleotides of the locker anchor may be complementary with the locker. Because the locker is longer than the locker anchor, this provides multiple advantages. Firstly, the longer length allows for a relatively high number of nucleotides to base pair with the locker target sequence, thereby forming a strong interaction and outcompeting a primer-such as a universal forward and/or reverse primer-that would otherwise interact with an overlapping primer binding site. Secondly, the longer length provides a password strand, i.e. the unlocker (described below) to bind which has a higher degree of complementarity compared to the locker anchor. This enables toehold mediated strand displacement wherein the locker and locker anchor no longer form a complex and instead the locker and password form a complex.

Hence, in an embodiment the length of the locker is between 20-100 nucleotides, preferably between 30-70 nucleotides, more preferably between 40-50 nucleotides. The locker anchor is a relatively short chain of nucleotides, from about 10-100 nucleotides, preferably 10-40 nucleotides, more preferably 10-25 nucleotides, most preferably 12-16 nucleotides.

In one embodiment according to the invention, the locker, locker anchor, and/or unlocker comprises or consists of DNA. Hence, these components are made up of the four canonical DNA nucleotides, i.e. adenine, thymine, cytosine and guanine.

In one embodiment, the biotin-binding agent is a tetrameric biotin-binding protein. It is advantageous to use a biotin-binding protein that can bind more than one biotin, such as a tetrameric biotin-binding protein, because it enables the locker complex and polynucleotide fragments to be localized (within the proteinosome) within proximity to each other. Without being bound by theory, due to the binding of four nucleotide components (lockers and/or polynucleotide fragments) to a single biotin-binding protein, localization may be improved as it prevents diffusion out of the proteinosome. It is further advantageous to use Tamavidin 2-HOT since streptavidin, avidin and/or tamavidin (non 2-HOT derivative) and the like are only partially resistant to the high temperatures used during PCR. Hence the heat-stable streptavidin analogue Tamavidin 2-HOT is preferably used. It may be contemplated to use non- or partially heat resistant biotin-binding protein, but this may be less preferred. However, it may be useful to inactivate biotin-binding function after removing the unlocker/locker complex, before continuing with amplification. It is understood that of course instead of using a biotin-label and biotin-binding agents, instead, other suitable labels and corresponding suitable label-binding agents may be contemplated that can function in the same way as the biotin-label and biotin-binding agent as described herein.

In one embodiment, when localizing the biotin-labeled double stranded polynucleotide fragments at least 5, 10, 20 or even 100 copies of all unique biotin-labeled double stranded polynucleotide fragments representing the nucleic acid-encoded dataset are distributed across the plurality of proteinosome compartments. Hence, the biotin-labeled double stranded polynucleotide fragments cover the nucleic acid encoded dataset 5, 10, 20 or even 100 times. It is advantageous to have a distribution of polynucleotide fragments as allows more efficient localization of fragments in proteinosomes. I.e. it avoids having to ensure that each proteinosome comprises 100% of all unique polynucleotide fragments whilst retaining the ability to recover the entire digital dataset as all fragments are present as a whole.

In an embodiment both of the strands of a double stranded fragment comprise a biotin label. This is highly advantageous as both strands of a double stranded polynucleotide fragment now can bind independently to a biotin-binding protein. The localization of both strand is therefore not dependent on the interaction with the complementary strand comprising the biotin label for localization thereof.

In one embodiment, on average 50-100%, preferably 75-100%, more preferably 90-100% of the biotin-labeled double stranded polynucleotide fragments representing the nucleic acid encoded dataset are localized in a single proteinosome compartment and wherein the biotin-labeled double stranded polynucleotide fragments are distributed over the proteinosome compartments thereby covering the nucleic acid-encoded dataset at least once. According to this and previous embodiments, across all proteinosome compartments the biotin-labeled double stranded polynucleotide fragments are distributed and at least one complete nucleic acid-encoded dataset is represented. Accordingly, the nucleic acid encoded dataset is represented at least once and hence the entire digital dataset is covered in full.

In one embodiment, the ratio of polynucleotide fragments to locker is at least 1:1, preferably 1:5, more preferably 1:10, most preferably 1:25. Of course, the number of lockers, i.e. number of molecules, is preferably in excess over the number of locker target sequences. Hence, in another embodiment, the ratio of the number of polynucleotide fragment strands comprising a locker target sequence to the number of lockers is of at least 1:1, preferably 1:5, more preferably 1:10, most preferably 1:25. In yet another embodiment the ratio of the number of locker target sequences present in the provided biotin-labeled double stranded polynucleotide fragments, to the number of lockers, is at least 1:1, preferably 1:5, more preferably 1:10, most preferably 1:25.

In yet another embodiment, the ratio of biotin-labeled double stranded polynucleotide fragments to locker or biotin-labeled double stranded polynucleotide fragments comprising the locker target sequence to locker is at least 1:1, preferably 1:5, more preferably 1:10, most preferably 1:25.

In any case, it is highly advantageous to have the locker in a concentration such that it substantially prevents amplification of substantially all polynucleotide fragments comprising the locker target sequence during PCR. It is advantageous to have an amount of locker that is equal or higher compared to the number of locker target sequences being present because this way substantially all locker target sequences can have a locker bound therewith and hence PCR amplification is blocked. The inventors have advantageously found that increasing amounts of locker, i.e. higher ratios, does not negatively influence the amplification of polynucleotide fragments lacking the locker target sequence.

In one embodiment the length of the unlocker is between 10-100 nucleotides, preferably between 10-50 nucleotides, more preferably between 15-30 nucleotides.

In one embodiment, the washing step of step k) is repeated at least once more (i.e. twice or more in total) to further remove locker strands that were not removed during the previous washing step.

In another embodiment, the proteinosome compartments comprise magnetic particles which are used to magnetically pull down the proteinosome compartments during washing. This has the advantage of improved separation of the proteinosome compartments from the locker bound to the unlocker. In yet a further embodiment, the proteinosomes are pulled down using the magnetic particles and in between the more than one washing step the proteinosomes are resuspended and subsequently pulled down using a magnet.

In a preferred embodiment, the interaction between the locker strand and the unlocker is performed between 0-32 degrees Centigrade, preferably between 5-30 degrees Centigrade, more preferably between 15-25 degrees Centigrade. It is preferred to not go beyond 32 degrees Centigrade as this is the lower critical solution temperature (LCST) the PNIPAm polymer comprised in the proteinosome. In another embodiment, wherein the polymer comprised in the proteinosome has an increased LCST, interaction between the locker strand and the unlocker is performed between 0-37 degrees Centigrade, preferably between 5-30 degrees Centigrade, more preferably between 15-25 degrees Centigrade. Such higher temperatures are contemplated. Hence, the interaction between the locker and unlocker is performed at temperatures below conventional PCR reactions but at temperatures which are compatible with the polymer used and it's LCST. The process of toehold mediated strand displacement is carried out without the participation of enzymes.

In an embodiment the unlocker is provided, in a ratio of locker to unlocker of at least 1:1, preferably 1:5, more preferably 1:10, most preferably 1:25. Regardless of exact ratios, in accordance with the invention, the locking and unlocking of polynucleotide fragments is based on the presence of a locker and unlocker or the absence of a locker and unlocker. Initially the locker should prevent amplification of polynucleotide fragments comprising the locker target, hence it is preferred that there is at least equal amount of locker to locker target sequences, preferably an excess of the locker. Subsequently, for toehold mediated strand displacement and removal of the locker, it is preferred that there is at least an equal amount of unlocker to locker, preferably an excess of the unlocker. Hence it is preferred that the amount of unlocker present in the mixture is equal to or exceeding the amount of locker in the mixture.

In one embodiment according to the invention, the amplified unlocked polynucleotide fragments comprises or consists of DNA and/or the biotin-labeled double stranded polynucleotide fragments comprises or consists of DNA and/or the amplified unlocked polynucleotide fragments comprises or consist of DNA. Hence, in such an embodiment, any of the physical nucleic acid fragments, be it polynucleotide fragments, biotin-labeled double stranded polynucleotide fragments or the amplified unlocked polynucleotide fragments comprise or consist of DNA.

In one embodiment the amplified unlocked polynucleotide fragments are sequenced to therewith obtain the polynucleotide sequences of the plurality of biotin-labeled double stranded polynucleotide fragments representing the nucleic acid-encoded dataset. To obtain the digital data from the amplified unlocked polynucleotide fragments sequencing is performed. Sequencing may be performed by any means known to the skilled person such as, but not limited to, Sanger sequencing, Next-generation sequencing (NGS) or high-throughput sequencing such as Illumina sequencing.

In an embodiment the the obtained polynucleotide sequences of the amplified unlocked polynucleotide fragments comprising the nucleic acid-encoded dataset are decoded and/or ordered by means of the indexing sequence to restore the digital dataset as provided in step a). To retrieve the digital dataset that was originally physically and molecularly encrypted, the sequences are decoded. The decoding involves transforming the nucleic acid-encoded sequence back to the digital dataset. The decoding algorithm may be the inverse of the encoding algorithm, i.e. the specific rules and procedures to transform digital dataset to nucleic acid code are used (in reverse) to decode. Other decoding algorithms are known in the art. Known decoding algorithms can tolerate dropouts ranging from approximately 5-20%. Hence, in an embodiment the obtained sequences do not cover all polynucleotide fragments but still allow the decoding of the obtained sequences and retrieving the digital dataset as provided.

In an embodiment, the indexing sequences comprised in the obtained polynucleotide sequences (which were, of course, present from the design stage) are used to correctly order said obtained sequences. Hence, such indexing sequences are highly advantageous as it allows either manual or automatic sorting of the plurality of polynucleotide sequences.

In another embodiment, the indexing sequences are used to correctly identify which sequences, in case of multiple datasets, originate from a single digital dataset in.

Elements of the invention as described above and advantageous embodiments thereto, can be envisioned as independent components that alone or in different combinations, are equally highly advantageous as well. Therefore, a locker complex comprising a locker and a locker anchor in accordance with the means and methods according to the invention alone is highly advantageous because such a complex may be provided independently as a way to lock molecularly encrypted nucleic acid-encoded data. Likewise, an unlocker may be provided independently, which is useful for unlocking locked molecularly encrypted nucleic acid-encoded data.

Herein and above, highly advantageous features as well as means and methods for obtaining physically molecularly encrypted nucleic acid-encoded data are provided. Hence in the present invention as described above, the physically molecularly encrypted nucleic acid-encoded data, and data system, obtainable by the methods above described, and e.g. such as described in example 1 are provided. Hence in one embodiment such a system is provided and comprises a plurality of double stranded polynucleotide fragments, a locker complex, and an unlocker, as defined herein, wherein the double stranded polynucleotide fragments and the locker complex (locker and locker and anchor) are combined in one container, and the unlocker is provided as a molecule in a separate container. Which physically encrypted nucleic acid-encoded data can be unlocked by providing an unlocker as described herein.

### Embodiments without Compartmentalization

Moreover, the invention provides for the advantageous concept of locking access physically molecularly encrypted nucleic acid-encoded data, with a locker, which may not necessarily require the advantageous means of compartmentalization utilizing e.g. biotin-labels and biotin-binding agents, such as described herein above and as described in example 1. Hence, it is understood that in the means and methods as described herein in accordance with the invention, the aspect related to biotin-labeling of (double stranded) polynucleotide fragments may not be included (i.a. not requiring a locker anchor) and/or not requiring proteinosomes. It is understood that these aspects are highly advantageous, but is also understood that lesser stringent security measures may still be sufficient. For example, because access to the containers with the physically molecular encrypted nucleic acid-encoded data, is physically restricted and operations to which the physically molecular encrypted nucleic acid-encoded data can be subjected can be restricted, as the containers are comprised within a specifically designed device with pre-defined unit operations. Such unit operations may comprise e.g. adding an unlocker to a mixture of a locker with (double stranded) polynucleotide fragments as described herein, and subsequently, separating unlocker bound with locker therefrom, to provide for unlocked (double stranded) polynucleotide fragments that can next be subjected to PCR-amplification.

Hence, means and methods in accordance with the invention, requiring the concept of a locker, a locker target sequence being comprised in a PCR-amplifiable nucleic acid-encoded data set, and an unlocker, already can provide for a highly useful physical molecular encrypted nucleic acid-encoded data system.

It is understood that in these embodiments without compartmentalization according to the invention, there is no need for a locker anchor as described herein and/or the locker with which the unlocker is to interact is not localized in proteinosome compartments. Hence, in the absence of a locker anchor and compartmentalization, the unlocker may not be not restricted in its design with regard to features related to toehold mediated strand displacement and/or proteinosome compartmentalization.

Advantageously, the unlocker may be selected to interact with the locker at any suitable temperature, as long it can provide for an appropriate binding specificity/selectivity between the locker and unlocker to allow for separation of the locker by means of the unlocker. Hence, preferably, the complementarity between the unlocker and locker preferably may be at least 10 or more nucleotides, up to over the full length of the locker, i.e. all nucleotides of the unlocker interact with (part of) the nucleotides of the locker. Preferably, the length of the unlocker is between 10-100 nucleotides, preferably between 10-50 nucleotides, more preferably between 15-30 nucleotides. Suitable temperatures to allow for interaction between unlocker and locker may be selected to be in the range of temperatures between 0 and 70 degrees Centigrade, more preferably between 15 and 50 degrees Centigrade, most preferably between 20 and 40 degrees Centigrade. It may be preferred to select a suitable temperature such that the double stranded polynucleotides that are present do not substantially denature, as such temperatures may allow for the locker (and unlocker) to hybridize with such denatured strands.

With regard to the aspects as described herein above in so far as these relate to primers, primer binding sites, locker and locker target sites and the like, and their interaction, in particular during PCR-amplification, with regard to their aspects that are not related to compartmentalization requirements as described above, such aspects may apply substantially equal to embodiments that do not require compartmentalization. Hence, the skilled person is well aware of how to providing for embodiments utilizing a suitable locker target nucleic acid sequence, suitable primer binding sites, to be comprised in polynucleotide sequences/fragments to provide for PCR-amplifiable polynucleotide fragments from suitable primers which can be locked thereof with a locker. Such suitable and non-limiting embodiments are described here below.

Accordingly, in another embodiment, physically molecularly encrypted nucleic acid-encoded data is provided comprising:
a) a plurality of double stranded polynucleotide fragments, comprising primer binding sites, said primer binding sites flanking nucleic acid encoded data
b) wherein at least a fraction of the double stranded polynucleotide fragments comprises one or more locker target sequences, wherein the locker target sequence(s) is/are located such that PCR amplification is blocked when a locker is bound to the locker target sequence, from the primer binding site sites for amplification of said double stranded polynucleotide fragments;
c) a locker, which is an oligonucleotide, having a modification that blocks amplification, optionally wherein the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide;
d) wherein the locker has sequence complementarity to the locker target in the double stranded polynucleotide fragments, and binds thereto, thereby preventing amplification of said double stranded polynucleotide fragments with primers from the primer binding sites; and
wherein the plurality of double stranded polynucleotide fragments and the locker are mixed.

Such physically molecularly encrypted nucleic acid-encoded data can subsequently be unlocked with an unlocker. Hence accordingly, in a further embodiment, of this embodiment, the use of an unlocker is provided, for unlocking said physically encrypted nucleic acid-encoded data, wherein the unlocker is provided with a tag and comprises an oligonucleotide sequence complementary with the locker, and wherein the unlocker is allowed to interact with the locker thereby forming a double stranded complex of the locker and the unlocker, which double stranded complex is subsequently separated from the plurality of double stranded polynucleotide fragments. Such use preferably comprises as tag a biotin-label, and magnetic beads with a biotin-binding agent are provided for separating the double stranded complex of the locker and the unlocker from the plurality of double stranded polynucleotide fragments.

Hence, it is understood that in accordance with these embodiments, the unlocker is to be provided with a tag that allows for subsequent separation of the unlocker, when bound with the locker, from the double stranded polynucleotide fragments. It may be contemplated to have the unlocker directly bound with a tag that allows for direct separation, e.g. the tag may be a (magnetic) bead. A tag may also be a molecule that allows for binding with a agent that can bind therewith, wherein said agent is e.g. bound with a (magnetic) bead. Such (magnetic) beads may allow for easy separation. For example, beads may be separated from a suspension via centrifugation. Advantageously, magnetic beads may be preferred, as these allow for convenient magnetic separation steps. A molecule, and agent that binds with said agent, may be for example biotin, and a biotin-binding agent respectively. A molecule, and agent that binds with said agent, may be for example a capture oligonucleotide sequence, and sequence complementary with said capture oligonucleotide sequence. Said biotin-binding agent, or said sequence complementary with said capture oligonucleotide sequence being advantageously conjugated with a (magnetic) bead to allow for easy separation of a double stranded locker-unlocker complex formed.

Hence, in order to arrive at such physically molecularly encrypted nucleic acid-encoded data, and unlocking thereof with an unlocker, the methods as described herein above that rely on biotin-labeled (double stranded) polynucleotide fragments (i.a. not requiring a locker anchor) and/or not proteinosomes such as described e.g. in example 1, can easily be adapted to provide for methods for providing such physically molecularly encrypted nucleic acid-encoded data, and unlocking thereof with an unlocker. Such an alternative embodiment is e.g. described in example 2 herein. Hence, the skilled person is well aware of providing for a suitable target nucleic acid sequence, and suitable primer binding sites, to be comprised in polynucleotide sequences/fragments to provide for PCR-amplifiable polynucleotide fragments, for which PCR-amplification can be locked for PCR-amplification by providing a locker (i.e. an oligonucleotide (substantially) complementary with the target nucleotide sequence), which can subsequently be unlocked with an unlocker. Such methods are described i.a. by the following embodiments 1-17 below:

### Embodiments 1-17

1. A method for physical molecular encryption of nucleic acid-encoded data, the method comprising the steps:
a) providing a digital dataset;
b) encoding the digital dataset to a nucleic acid code, thereby designing a nucleic acid-encoded dataset, wherein the nucleic acid-encoded dataset comprises a plurality of polynucleotide sequences, preferably of between 50-500 nucleotides, preferably 100-250 nucleotides, more preferably 150-200 nucleotides, wherein each of the polynucleotide sequences further comprise a forward and reverse primer binding site at the ends of the polynucleotide sequences that allows for subsequent PCR amplification wherein up to 100% of the individual polynucleotide sequences comprise at least one locker target sequence wherein the locker target sequence is located such that PCR amplification is blocked when a locker is bound to the locker target sequence, optionally wherein the locker target sequence and/or the primer binding site(s) of the polynucleotide sequence is/are part of the nucleic acid-encoded dataset, and/or optionally wherein the polynucleotide sequences comprise an indexing sequence;
c) synthesizing the designed plurality of polynucleotide sequences to provide for polynucleotide fragments, wherein the polynucleotide fragments are synthesized as single stranded polynucleotide fragments and subsequently made double stranded polynucleotide fragments or synthesized as double stranded polynucleotide fragments, preferably comprising an amplification step to provide for a desired concentration of double stranded polynucleotide fragments;
d) therewith providing for double stranded polynucleotide fragments representing the nucleic acid-encoded dataset.
2. The method according to embodiment 1, further comprising the steps of:
e) providing a locker, wherein the locker is an oligonucleotide, wherein the locker comprises a sequence complementary to the locker target sequence, wherein the locker comprises a modification that blocks amplification, optionally wherein the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide,
to therewith provide for double stranded polynucleotide fragments representing the nucleic acid-encoded dataset and locker, which are preferably mixed, to provide for physically molecular encrypted nucleic acid-encoded data, wherein PCR amplification of the double stranded polynucleotide fragments is repressed in the double stranded polynucleotide fragments comprising the locker target sequence by binding of the locker.
3. A method comprising unlocking of physical molecular encrypted nucleic acid-encoded data, comprising the steps of:
i) providing double stranded polynucleotide fragments representing the nucleic acid-encoded dataset and a locker, which are mixed, as obtained in embodiment 2;
j) providing an unlocker, wherein the unlocker comprises an oligonucleotide sequence complementary to the locker, and wherein the unlocker comprises a tag;
k) mixing the unlocker with the double stranded polynucleotide fragments representing the nucleic acid-encoded dataset and the locker;
l) allowing the unlocker to interact with the locker therewith forming a double stranded complex of the locker and the unlocker,
m) separating the double stranded complex of the locker and the unlocker from the double stranded polynucleotide fragments by means of the tag;
n) amplifying the double stranded polynucleotide fragments obtained in step m) to therewith obtain amplified unlocked polynucleotide fragments, preferably using a forward universal primer and a reverse universal primer targeting the forward and reverse primer binding sites.
4. The method according to any of embodiments 1-3, wherein a plurality of digital datasets is provided wherein each of the digital datasets is encoded to individual nucleic acid codes, thereby designing a plurality of individual nucleic acid-encoded datasets.
5. The method according to any of embodiments 1-4, wherein a plurality of digital datasets is provided wherein each of the digital datasets is encoded to individual nucleic acid codes, thereby designing a plurality of individual nucleic acid-encoded datasets, wherein the individual nucleic acid-encoded datasets each comprise a plurality of polynucleotide sequences comprising unique indexing sequences for decoding, ordering of polynucleotide sequences and/or identifying the nucleic acid-encoded dataset to which the polynucleotide sequences belong.
6. The method according to any of embodiments 1-5, wherein the nucleic acid-encoded dataset is a DNA encoded dataset, and the double stranded polynucleotide fragments are double stranded DNA fragments.
7. The method according to any of embodiments 1-6, wherein the locker target sequence is overlapping with one of the primer binding sites.
8. The method according to any of embodiments 1-7, wherein the polynucleotide sequences representing the nucleic acid-encoded dataset comprise more than one unique locker target sequence.
9. The method according any of embodiment 2-8, wherein the locker, and/or unlocker comprises DNA.
10. The method according to any of embodiments 2-9, wherein the locker is provided in a ratio of polynucleotide fragments to locker is of at least 1:1, preferably 1:5, more preferably 1:10, most preferably 1:25, preferably wherein the ratio of locker target sequences comprised in polynucleotide fragments to locker to is at least 1:1, preferably 1:5, more preferably 1:10, most preferably 1:25.
11. The method according to any of embodiment 3-10, wherein the tag is a biotin-label.
12. The method according to any of embodiment 11, wherein the step of separating the double stranded complex by means of the tag, comprises a separating step utilizing a biotin-binding agent.
13. The method according to embodiment 12, wherein a magnetic bead is provided with the biotin-binding agent, wherein the biotin-binding agent preferably is a biotin-binding protein, such as streptavidin, and the separation step involves magnetic bead separation.
14. The method according to any of embodiment 3-13, wherein the amplified unlocked polynucleotide fragments comprises DNA or wherein the amplified unlocked polynucleotide fragments consist of DNA.
15. The method according to any of embodiments 3-14, wherein the unlocker is provided in a ratio of locker to unlocker of at least 1:1, preferably 1:5, more preferably 1:10, most preferably 1:25.
16. The method according to any of embodiment 3-15, wherein the amplified unlocked polynucleotide fragments are sequenced to therewith obtain the polynucleotide sequences of the plurality of biotin-labeled double stranded polynucleotide fragments representing the nucleic acid-encoded dataset.
17. The method according to embodiment 16, wherein the obtained sequences of the amplified unlocked polynucleotide fragments comprising the nucleic acid-encoded dataset are decoded to restore the digital dataset as provided in step a), which decoding and restoring comprises ordering, optionally by means of the indexing sequence.

Hence, accordingly, in these embodiments, the invention provides for a physically molecularly encrypted nucleic acid-encoded data system, comprising a plurality of double stranded polynucleotide fragments, and a locker as defined above, wherein the mixture of double stranded polynucleotide fragments and the locker are combined in one container, and the unlocker as defined in these embodiment is provided in a separate container.

### EXAMPLES

### Example 1

The present invention is further elucidated based on the examples below which are illustrative only and are not to be construed to be limiting to the present invention.

### Example 1.1: Locker-Controlled PCR Repression

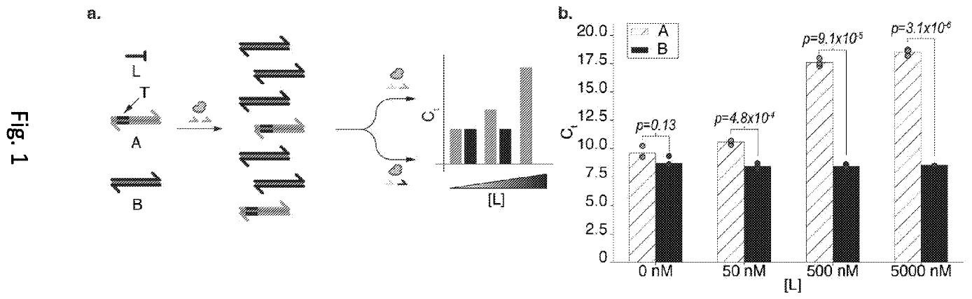

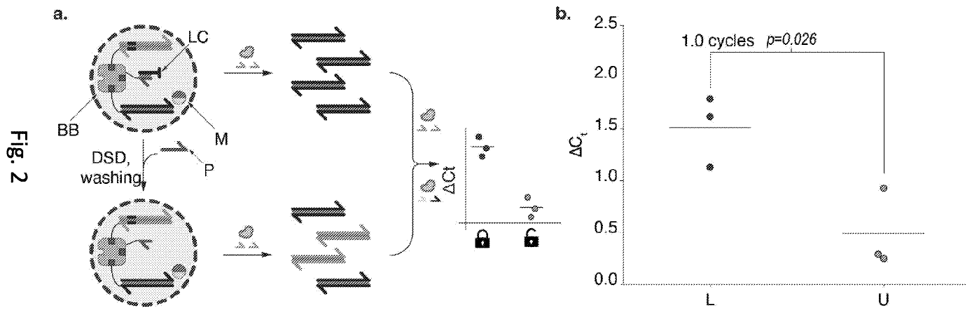

The effects of increasing the concentration of the locker, hereafter interchangeably referred to as ‘locker’ or ‘locker strand’, on PCR based amplification of double-stranded DNA (dsDNA) polynucleotide fragment templates was tested. The minimal concentration of locker strand required for blocked PCR was determined by designing two dsDNA templates (template A: A1A2 and template B: B1B2) that can be amplified by a set of shared universal primers (UFW and URV). One of the two templates (A1A2) was designed to be blocked by the locker strand (L1). Additionally, a secondary set of orthogonal primers (AFW & ARV and BFW & BRV) specific for each template was designed to enable quantification after locked PCR using qPCR. For a schematic overview of the experiment see FIG. 1A. For sequences used in Example 1.1, see the Table 1 below.

TABLE 1
DNA sequences used in Example 1.1

| | SEQ- | | | |
|---|---|---|---|---|
| | ID | | Length | |
| Name | NR. | Sequence | (#nt) | Modification |
| A1 | 1 | TGTCTCACTAGGTCCTCTCAGCTCTTC | 168 | |
| | | CCACCACCATTGCTTCTTCTTCCTTCTG | | |
| | | AGTCTAACCACCACCATTGCCTTATCTA | | |
| | | CCATTCTGAGTCTTACCACCACCATTG | | |
| | | CCATTTCATTATCTACGAGTCTATCCAC | | |
| | | CACCATTGCAGGAGTTTCAGTTTCGAC | | |
| | | GC | | |
| A2 | 2 | GCGTCGAAACTGAAACTCCTGCAATGG | 168 | |
| | | TGGTGGATAGACTCGTAGATAATGAAA | | |
| | | TGGCAATGGTGGTGGTAAGACTCAGAA | | |
| | | TGGTAGATAAGGCAATGGTGGTGGTTA | | |
| | | GACTCAGAAGGAAGAAGAAGGCAATG | | |
| | | GTGGTGGGAAGAGCTGAGAGGACCTA | | |
| | | GTGAGACA | | |
| B1 | 3 | TGTCTCACTAGGTCCTCTCATTCCGAG | 168 | |
| | | GGAGATCTACACTGCTCGTCTTAATATA | | |
| | | CGGAATAACCAGCCCGCGGCTCCAAA | | |
| | | CCGACTAGATTCTTTCTCGAGTTATCCA | | |
| | | CATACATCGGTCTTATATTGTTGGTGAT | | |
| | | ACCCAAACAATAGGAGTTTCAGTTTCG | | |
| | | ACGC | | |
| B2 | 4 | GCGTCGAAACTGAAACTCCTATTGTTT | 168 | |
| | | GGGTATCACCAACAATATAAGACCGAT | | |
| | | GTATGTGGATAACTCGAGAAAGAATCT | | |
| | | AGTCGGTTTGGAGCCGCGGGCTGGTT | | |
| | | ATTCCGTATATTAAGACGAGCAGTGTA | | |
| | | GATCTCCCTCGGAATGAGAGGACCTAG | | |
| | | TGAGACA | | |
| UFW | 9 | TGTCTCACTAGGTCCTCTCA | 20 | |
| URV | 10 | GCGTCGAAACTGAAACTCCT | 20 | |
| AFW | 11 | CCACCATTGCCTTCTTCTTC | 20 | |
| ARV | 12 | GGATAGACTCGTAGATAATG | 20 | |
| BFW | 13 | CCGAGGGAGATCTACACTGC | 20 | |
| BRV | 14 | GTATCACCAACAATATAAGAC | 21 | |
| L1 | 15 | CACTAGGTCCTCTCAGCTCTTCCCACC | 43 | 3′ inverted dT |
| | | ACCATTGCCTTCTTCT | | |

The effects of increasing locker concentrations using these two templates and the designed primers and locker strand was quantified. Equal concentration mixtures of the two templates were amplified using standard, well-mixed, PCR conditions with increasing concentrations of the locker strand (see Methods: PCR). Following this initial amplification, the mixtures were purified, and the relative concentrations of both strands were determined using qPCR (see Methods: qPCR). Without the presence of the locker strand no significant differences in relative concentrations or threshold cycle (Ct) values were observed. In contrast, at locker concentration above 50 nM, such as for example 10 100 nM, 200 nM, 500 nM, 1000 nM or 5000 nM, significant differences in the relative concentrations of the two strands after locked PCR are observed as well as significant differences in threshold cycle (Ct) values of the two strands (FIG. 1B and Table 2). The presence of locker strand doesn't negatively affect the amplification of the non-locked strand (p=0.76, one-way ANOVA). Collectively, these results indicate that even low concentrations of locker strands can lead to PCR repression without affecting, such as negatively repression of other templates that are present.

TABLE 2
Ct values FIG. 1B

| | | A | B |
|---|---|---|---|
| 0 | nM | 10.25 | 9.36 |
| | | 9.31 | 8.62 |
| | | 9.27 | 8.25 |
| 50 | nM | 10.37 | 8.73 |
| | | 10.66 | 8.44 |
| | | 10.71 | 8.23 |
| 500 | nM | 17.28 | 8.62 |
| | | 17.98 | 8.44 |
| | | 17.53 | 8.34 |
| 5000 | nM | 18.74 | 8.91 |
| | | 18.61 | 8.51 |
| | | 18.21 | 8.26 |

### Example 1.2: Locking and Unlocking of Proteinosome-Localized DNA Strands

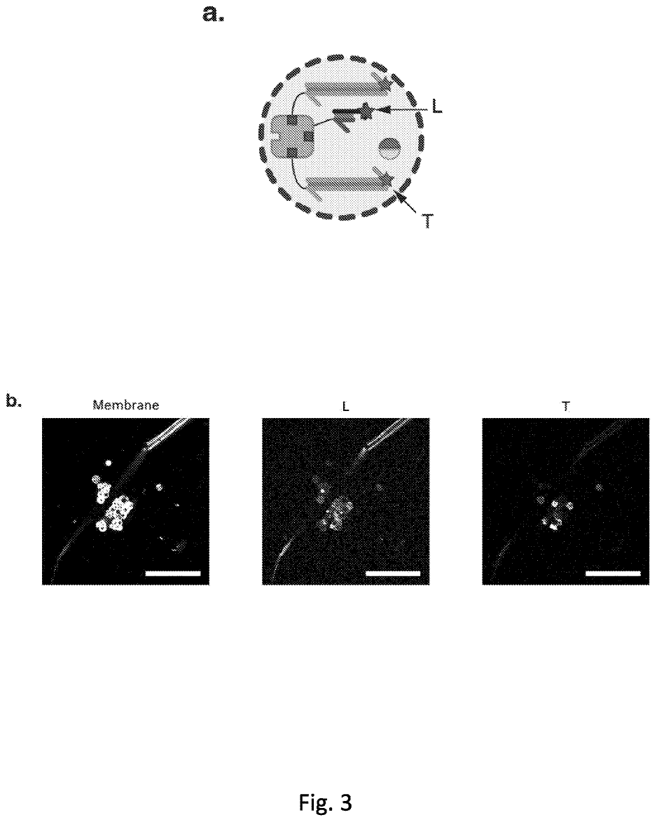

The locking and unlocking of proteinosome-localized templates (i.e. dsDNA polynucleotide fragments according to the invention) was demonstrated (FIG. 2A). Similar to the Example 1.1, two dsDNA templates (template A: A3A4 and template B: B3B4) were employed that share a universal primer pair (UFW and URV) and can each be amplified using orthogonal pairs of primers (AFW & ARV and BFW & BRV) to quantify the relative product concentrations post-amplification using qPCR. The dsDNA templates were localized inside proteinosomes containing, preferably, 4 μM of a biotin-binding protein such as Tamavidin 2-HOT and by labeling the dsDNA with biotin (Methods). The biotinylated locker strand (L1) was colocalized (see Methods: localizing DNA in proteinosomes and FIG. 3) inside the proteinosomes with the dsDNA template to skew PCR-based amplification inside proteinosomes by hybridizing it to a, preferably shorter, complementary, biotin-labeled locker anchor strand (L2), therewith forming a locker complex. Using a, preferably longer, complementary unlocker, also referred to as a password (P) and hereafter interchangeably referred to as ‘unlocker’, ‘unlocker strand’, ‘password’ or ‘password strand’, the locker strand was displaced from the proteinosomes via toehold-mediated DNA strand displacement. For sequences used in Example 1.2, see Table 3.

TABLE 3
DNA sequences used in Example 1.2

| | SEQ- | | Length | |
|---|---|---|---|---|
| Name | ID | Sequence | (#nt) | Modification |
| A3 | 5 | TGTCTCACTAGGTCCTCTCAGCTCTTC | 179 | |
| | | CCACCACCATTGCCTTCTTCTTCCTTC | | |
| | | TGAGTCTAACCACCACCATTGCCTTAT | | |
| | | CTACCATTCTGAGTCTTACCACCACCA | | |
| | | TTGCCATTTCATTATCTACGAGTCTATC | | |
| | | CACCACCATTGCAGGAGTTTCAGTTTC | | |
| | | GACGCAAAAAAAAAAA | | |
| A4 | 6 | TTTTTTTTTTTGCGTCGAAACTGAAACT | 179 | 5′ biotin |
| | | CCTGCAATGGTGGTGGATAGACTCGTA | | |
| | | GATAATGAAATGGCAATGGTGGTGGTA | | |
| | | AGACTCAGAATGGTAGATAAGGCAATG | | |
| | | GTGGTGGTTAGACTCAGAAGGAAGAA | | |
| | | GAAGGCAATGGTGGTGGGAAGAGCTG | | |
| | | AGAGGACCTAGTGAGACA | | |
| B3 | 7 | TGTCTCACTAGGTCCTCTCATTCCGAG | 179 | |
| | | GGAGATCTACACTGCTCGTCTTAATAT | | |
| | | ACGGAATAACCAGCCCGCGGCTCCAA | | |
| | | ACCGACTAGATTCTTTCTCGAGTTATC | | |
| | | CACATACATCGGTCTTATATTGTTGGT | | |
| | | GATACCCAAACAATAGGAGTTTCAGTT | | |
| | | TCGACGCAAAAAAAAAAA | | |
| B4 | 8 | TTTTTTTTTTTGCGTCGAAACTGAAACT | 179 | 5′ biotin |
| | | CCTATTGTTTGGGTATCACCAACAATA | | |
| | | TAAGACCGATGTATGTGGATAACTCGA | | |
| | | GAAAGAATCTAGTCGGTTTGGAGCCG | | |
| | | CGGGCTGGTTATTCCGTATATTAAGAC | | |
| | | GAGCAGTGTAGATCTCCCTCGGAATGA | | |
| | | GAGGACCTAGTGAGACA | | |
| UFW | 9 | TGTCTCACTAGGTCCTCTCA | 20 | |
| URV | 10 | GCGTCGAAACTGAAACTCCT | 20 | |
| AFW | 11 | CCACCATTGCCTTCTTCTTC | 20 | |
| ARV | 12 | GGATAGACTCGTAGATAATG | 20 | |
| BFW | 13 | CCGAGGGAGATCTACACTGC | 20 | |
| BRV | 14 | GTATCACCAACAATATAAGAC | 21 | |
| L1 | 15 | CACTAGGTCCTCTCAGCTCTTCCCACC | 43 | 3′ inverted dT |
| | | ACCATTGCCTTCTTCT | | |
| L2 | 16 | AGGGAAGAGCTGAGA | 15 | 5′ biotin |
| P | 17 | AGGGAAGAGCTGAGAGGACCTAG | 23 | |

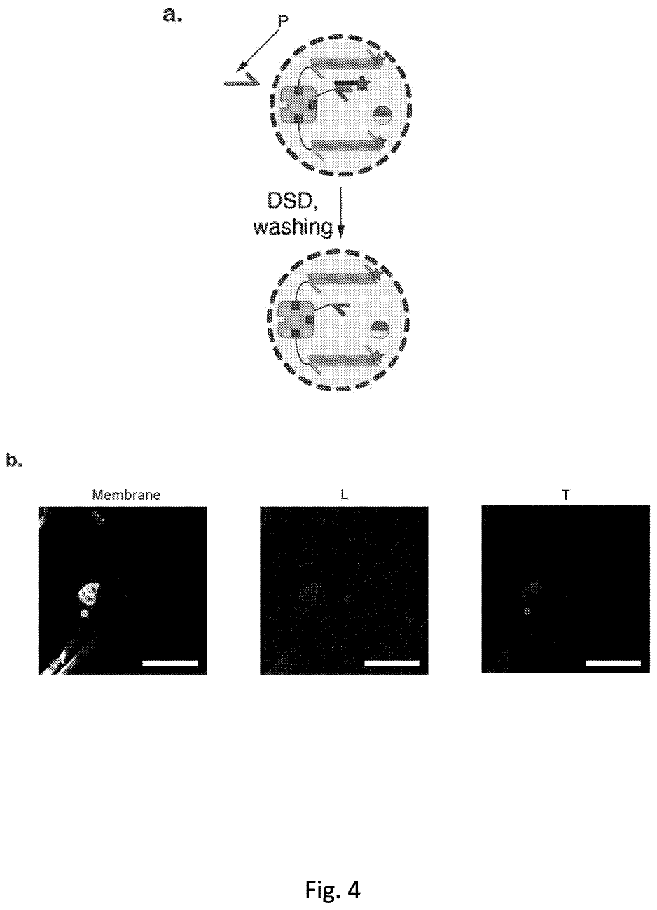

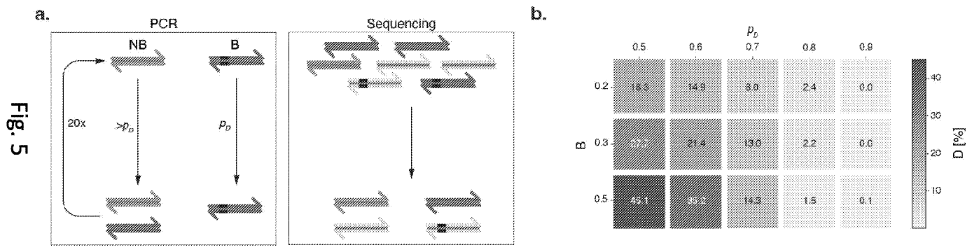

Subsequently, and preferably, washing was performed to remove the newly formed locker-unlocker complex (i.e. an unlocked complex) from the proteinosome-localized template dsDNA (FIG. 4), enabling even PCR-based amplification. Locked and unlocked proteinosomes were amplified using the universal primer pair and amplified products were retrieved by PCR purification (see Methods: PCR). Using qPCR, the relative fractions of both (A3A4 and B3B4) dsDNA templates were determined and used to calculate the differences in threshold cycle (ΔCt; Table 4) as a measure of amplification skewing, a statistically significant difference of 1.0 PCR cycle was measured for templates loaded in an equal ratio (FIG. 2B).

TABLE 4
Ct values FIG. 2B

| | Template A | Template B | ΔCt |
|---|---|---|---|
| Locked | 28.58 | 26.79 | 1.79 |
| | 23.46 | 21.84 | 1.62 |
| | 23.23 | 22.10 | 1.13 |
| Unlocked | 23.48 | 23.23 | 0.25 |
| | 23.73 | 22.80 | 0.93 |
| | 23.67 | 23.38 | 0.29 |

### Example 1.3 Simulating File-Level Data Locking

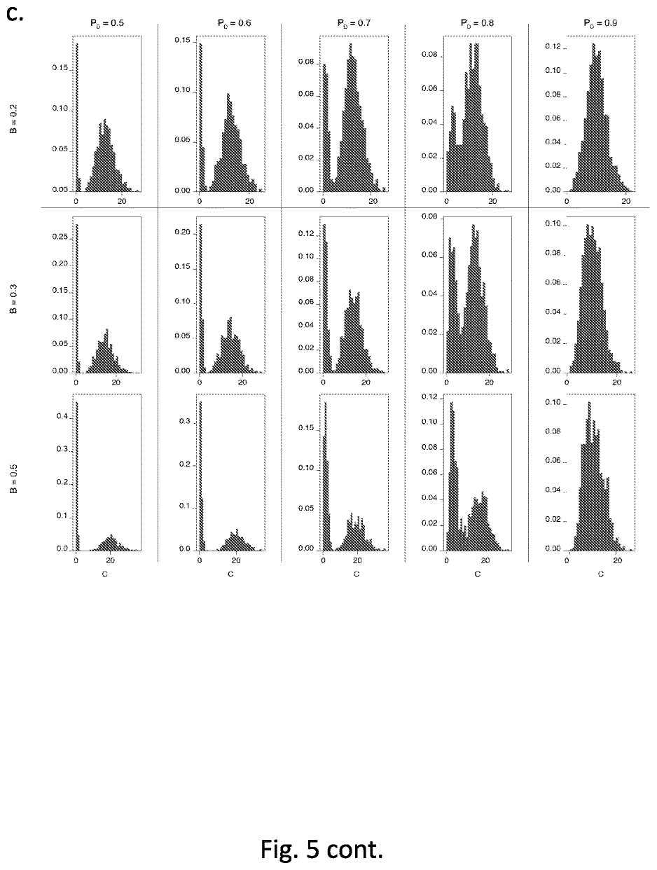

The effects of locking and unlocking of DNA-encoded data using a locker strand on a filesized DNA pool was investigated by modeling a hypothetical data-locking experiment (see Methods: Modelling locking and unlocking of DNA-encoded data and FIG. 5A). PCR-based DNA amplification is a stochastic process where not every strand is duplicated in each cycle. To include this behavior, PCR is modeled as a binomial process during which each DNA molecule is duplicated with a doubling chance pD for each cycle. By varying the value of pD between the blocked and non-blocked strands (i.e. strands comprising a locker target and strands lacking a locker target, respectively) biassing is introduced. During data decoding, PCR-amplified DNA strands are sequenced, which was simulated as a random sampling process from the total population of DNA strands.

Using the stochastic model, the effects of pD for blocked strands, and the fraction of blocked strands on sequence dropout after sequencing was studied. A file consisting of 1000 strands was simulated, all assumed to be present in equal numbers before (in silico) PCR-based amplification. Furthermore, for the purpose of the simulation it was assumed that unblocked strands have a 95 percent (pD=0.95) chance of being amplified during each of the 20 simulated PCR cycles. Finally, for all simulations, the average coverage was set to 10×. The effects of pD and the blocked strand fraction the value of pD for blocked strands was set to 0.5, 0.6, 0.7, 0.8, or 0.9 and the fractions of blocked strands were set to 0.2, 0.3, and 0.5. Based on the outcome of the simulations (FIG. 5B), a value of pD below 0.8 leads to a dropout increase and bimodal coverage distributions (FIG. 5C)

### Materials:

2-Ethyl-1-hexanol (Sigma, 98%); 1-(3-dimethylaminopropyl)-3-ethylcarbodiimide HCl (EDC, Carbosynth); 1,6-diaminohexane (Sigma, 98%); PEG-bis (N-succinimidyl succinate) (Mw=2000, Sigma); DyLight™ 405 NHS ester (ThermoFisher); bovine serum albumin (heat shock fraction, pH 7, ≥98%, Sigma); Tamavidin 2-HOT, recombinant (Wako Chemicals or expressed in-house); DynaBead M270 amine (Invitrogen); 1M MgCl2 (Invitrogen); 1M Tris pH 8.0 RNase-free (Invitrogen); 5M NaCl (Invitrogen); EvaGreen (Biotium); KAPA HiFi HotStart PCR Kit (Roche) were used as received. All other chemicals used were purchased from Sigma. Enzymes were purchased from New England Biolabs unless otherwise noted.

### Methods:

### Synthesizing BSA-NH2/PNIPAm Nanoconjugates

Cationized BSA (BSA-NH2) was synthesized according to a previously reported method23. Typically, a solution of diaminohexane (1.5 g, 12.9 mmol in 10 ml of MilliQ water) was adjusted to pH 6.5 using 5 M HCl and added dropwise to a stirred solution of BSA (200 mg, 3 μmol in 10 ml of MilliQ water). The coupling reaction was initiated by adding 100 mg of 1-(3-dimethylaminopropyl)-3-ethylcarbodiimide HCl (EDC) immediately and then another 50 mg after 5 h. If needed, the pH value was readjusted to 6.5 and the solution was stirred for another 6 h and then centrifuged to remove any precipitate. The supernatant was dialyzed (Medicell dialysis tubing, molecular weight cutoff (MWCO) of 12-14 kDa) overnight against MilliQ water and freeze-dried.

End-capped mercaptothiazoline-activated PNIPAm (Mn=13,284 g mol⁻¹, 4 mg in 5 ml of MilliQ water) was synthesized and added to a stirred solution of BSA-NH2 (10 mg in 5 ml of PBS buffer at pH 8.0). Molecular weight and polydispersity of activated PNIPAm were determined using GPC. The solution was stirred for 10 h and then purified using a centrifugal filter (Millipore, Amicon Ultra, MWCO of 50 kDA) and freeze-dried. After freeze-drying, the obtained BSA-NH2/PNIPAm conjugate was characterized by MALDI-MS and zeta potentiometry).

Dylight405-labeled BSA-NH2/PNIPAm conjugates were prepared using the same method, except that labeled BSA was used as the starting material.

Labeling BSA with Fluorescent Dyes

BSA was labelled with DyLight 405 as follows: 30 mg of BSA was dissolved in 6 mL of 50 mM sodium carbonate buffer (pH 9). 1 mg of DyLight 405 NHS ester was dissolved in 100 μL of DMF and added to the stirred BSA solution. The solution was stirred for 2 h, purified by dialyzing (Medicell dialysis tubing, MWCO 12-14 kDa) overnight against MilliQ water, and freeze-dried. Using UV-Vis spectrophotometry the average number of dyes per protein was determined; on average 1.4 dyes per BSA molecule but more or less dyes per BSA molecule may be contemplated.

### Preparing Tamavidin 2-HOT Containing Proteinosomes

Proteinosomes containing Tamavidin 2-HOT and magnetic particles (DynaBead M-270 amine) were prepared similarly to previous descriptions for Streptavidin-containing proteinosomes (Joesaar et al., Nature Nanotech. 14, 369-378). BSA-PNIPAm nanoconjugates (6 mg/mL total, 1 mg/ml of which were fluorescently labelled) were mixed with 4 μM Tamavidin 2-HOT and 4 mg/mL DynaBeads in 7.5 μL aqueous phase; 0.4 mg of PEG-bis(N-succinimidyl succinate) (Mw=2,000) was dissolved in 7.5 μL 50 mM sodium carbonate buffer (pH 9) and added to the mix which was then briefly vortexed. Next, 300 μL 2-Ethyl-1-hexanol was added, followed by vortexing and manual shaking to yield the Pickering emulsion. The resulting mixture was left at room temperature for 2 hours to crosslink nanoconjugates. The oil phase was removed by pipetting away the upper oil layer and 300 μL 70% ethanol was added to resuspend the sediment. The dispersion was then sequentially dialyzed (SpectraPor 4 Dialysis membrane 12-14 kD) against 70% and 50% ethanol for 2 h each and finally overnight against Milli-Q water to yield proteinosomes in water. Proteinosomes were then stored at 4° C. for later use.

### DNA Oligonucleotide Synthesis

All DNA oligonucleotides were purchased from Integrated DNA Technologies (IDT). Modified oligonucleotides were purchased with HPLC purification, non-modified oligonucleotides were purchased desalted. Stock solutions (100 μM and 10 μM) were made using nuclease-free TE buffer (10 mM Tris, 0.1 mM EDTA, PH 7.5; Integrated DNA Technologies) and stored at −30° C.

Preparing Double-Stranded DNA with Biotin of Fluorophores

Double-stranded complexes consisting of strands shorter than 100 nucleotides were formed by thermal annealing. Biotinylated strands were mixed with non-biotinylated strands at 12 and 10 μM, respectively, and heated to 95° C. in a thermocycler for 3 minutes. The samples were subsequently cooled to room temperature at a rate of −0.5° C./min.

Double-stranded DNA strands longer than 100 base pairs, with fluorescent and/or biotin modifications, were prepared from either single-stranded ultramer templates or double-stranded gBlocks and modified primers using PCR since these constructs could not be directly ordered. Typically, reactions were performed at the 100-μL scale using 5 μL (1 nM) diluted template, primers (0.5 M each), and KAPA HiFi HotStart polymerase. The amplification protocol was as follows: denaturing at 95° C. for 3 minutes, denaturing at 98° C. for 20 seconds, annealing at 65° C. for 15 seconds, and extending at 72° C. for 15 seconds. The second denaturing, annealing, and extending steps were repeated 16 times followed by a final extension at 72° C. for 30 seconds before cooling down to 4° C. The resulting amplicons were then purified using the Qiagen PCR extraction kit following the manufacturer's instructions.

### Localizing DNA in Proteinosomes

Biotin-labeled DNA was initially localized in 10 mM Tris (pH 8.0) with 11.5 mM MgCl₂ and 0.1% vol/vol Tween-20. Typically, 10 μL of proteinosome-containing solution was added to 5 μL 4× buffer solution and 5 μL of DNA to be localized. The mixture was kept at 4° C. overnight. The following day, 500 μL wash buffer, consisting of 10 mM Tris (pH 8.0), 1 M NaCl, 11.5 mM MgCl2, and 0.1% vol/vol Tween-20, was added, left at 4° C. overnight, and removed the following day. Secondary washing steps were performed similarly to the first except that no overnight step was used. Instead, proteinosomes were separated from the solution by placing the mixture in a magnetic separation rack (DynaMag, Invitrogen) for 3 minutes, after which the supernatant was removed by pipette.

### Statistics

All results reporting statistical values were from independent triplicates. Analysis was performed using Python's SciPy (Python 3.6.5, SciPy version 1.1.0) library. Welch's two-sided t-test was used to compare two populations. Statistical significance between more than two samples was determined using Oneway Analysis of Variance (ANOVA). Only values of p<0.05 were considered statistically significant.

### PCR

The total reaction volume was 25 UL and consisted of KAPA HiFi HotStart, 0.5 μM primers, and 2.5 μL proteinosome solution. Thermocycling was performed in a T1000 Touch thermocycler (Bio-Rad). Following initial denaturation for 3 minutes at 95° C., 12 cycles of denaturation at 98° C. for 20 seconds, annealing at 65° C. for 15 seconds, and extension at 72° C. for 15 seconds were performed, followed by a final extension at 72° C. for 30 seconds before cooling down to 4° C. Double-stranded DNA products were purified from the reaction mixture using a Qiagen PCR extraction kit following the manufacturer's instructions.

qPCR

Quantitative PCR was performed using CFX96 Touch Real-Time PCR Detection System (Bio-Rad). The total reaction volume was 25 UL and consisted of KAPA HiFi HotStart, 0.5 μM primers, 1× EvaGreen, and 2 μL purified DNA. Initial denaturation was set to 3 minutes at 95° C., and then 40 denaturation cycles at 98° C. for 20 seconds, annealing at 65° C. for 15 seconds, and extension at 72° C. for 15 seconds were performed, followed by a final extension at 72° C. for 30 seconds before cooling down to 4° C. Fluorescence was measured during each annealing step. To prevent evaporation, the plate was sealed with a transparent plate sealer. CFX Maestro Software (Bio-Rad) was used to perform baseline correction and calculate threshold cycles (Ct).

### Confocal Microscopy

Fluorescence data were acquired using a confocal laser scanning microscope (CLSM, Leica SP8) equipped with solid-state lasers (405 nm for DyLight405, 552 nm for Cy3, 638 nm for Cy5) and a hybrid detector in photon-counting mode. The measurements were performed with a ×10/0.40 numerical aperture (NA) (1.55×1.55 mm² field of view, 7 μm slice thickness) at a resolution of 512×512 pixels.

### Modelling Locking and Unlocking of DNA-Encoded Data

A stochastic model of PCR and sequencing for blocked and unblocked DNA was implemented in Python 3.6.5 and Numpy 1.21.5. As a first step 1000 DNA strands are initialized as unique numbers, serving as barcodes. Barcodes are then randomly assigned a blocked or unblocked state based on the “fraction blocked” parameter. For each barcode 20 PCR cycles were then performed using the following formula for the number of molecules in each cycle j:

$$n j = n j - 1 + B ( n j - 1 , pD )$$

Where nj is the number of molecules in the j^{th} cycle; B (nⱼ₋₁, P) is a binomially distributed random variable with nⱼ₋₁ molecules, and PD is the probability of a successful amplification. For unblocked strands, PD was set to 0.95, whereas for blocked strands it varied. After simulated PCR strands were randomly down sampled to a fixed coverage number to simulate NGS.

### Example 2

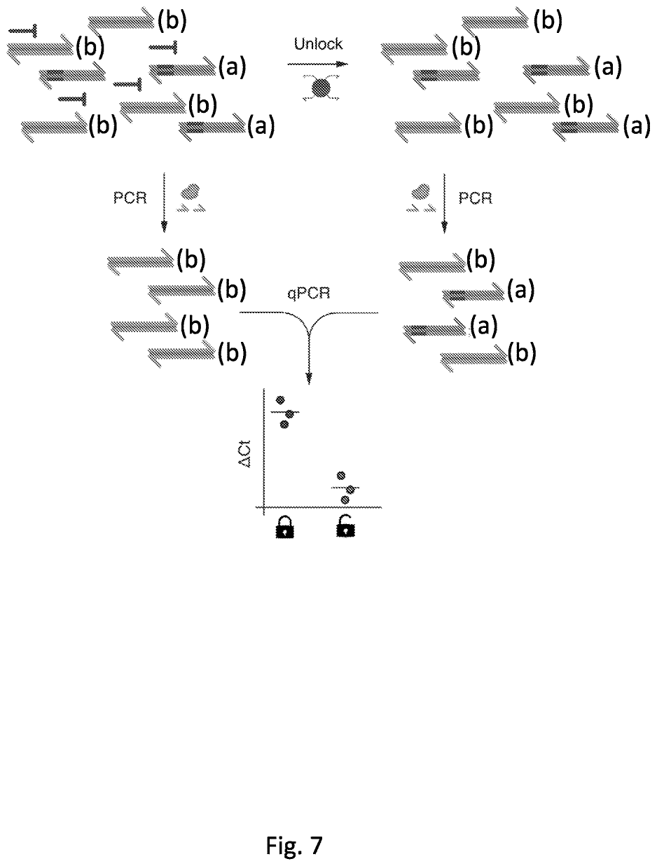

In view of the above means and methods, building on the advantageous observations that a locker strand can prevent PCR-based amplification when at least one of the two templates of double stranded polynucleotide fragments contains a locker target sequence we investigated if a locker could subsequently be removed from a mixture using instead magnetic separation (schematically depicted in FIG. 7). This method no longer requiring a locker anchor nor compartmentalization. To this end, means and methods as described above, i.a. such as in example 1.1 and table 1.1, were suitably adapted for this example. In addition to A1/A2, B1/B2, and UFW/URV, AFW/ARV, BFW/BRV and L1, provided as listed in table 1, a password strand P2 was designed as the reverse compliment of the locker strand to provide for binding between the locker and unlocker. This newly designed password strand, i.e. unlocker, was biotinylated, i.e. provided with a tag, to allow for facile binding to micrometer-sized magnetic particles coated with streptavidin beads to provide for beads with an unlocker. In this example, unlocker P2 has the sequence:

AAGAAGAAGGCAATGGTGGTGGGAAGAGCTGAGAGGACCTAGTG

with a length of 44 and a 5′ biotin label (SEQ ID NO. 18). This newly designed unlocker was fully complementary with the locker sequence L1. The addition of unlocker bound with magnetic beads to a mixture containing double stranded polynucleotide fragments and a locker strand results in hybridization between the locker and the password strands. Since the unlocker (i.e. password) oligonucleotides are bound with the magnetic particles the locker-unlocker complex can be easily removed from the mixture using magnetic pulldown.

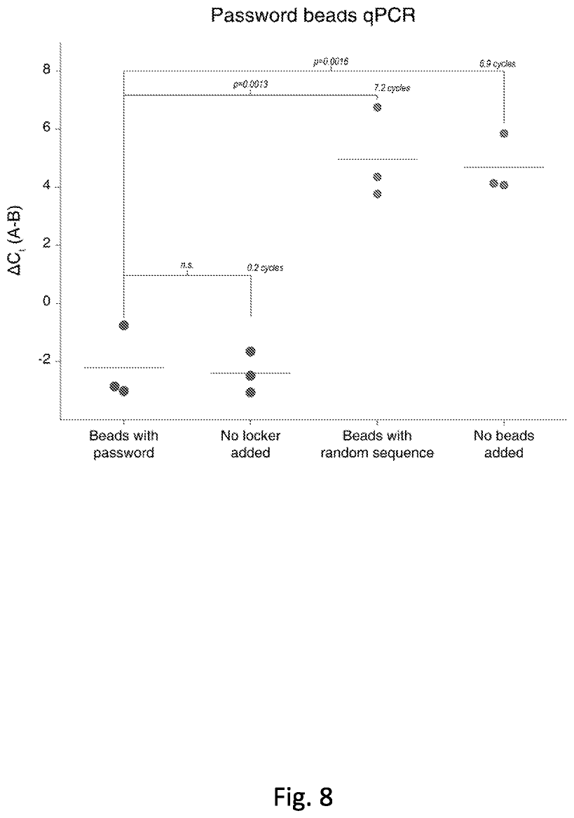

Similarly, and appropriately adapted to as above, double stranded templates A and B were mixed and 5 μM locker strand was added to this mixture. Subsequently password beads (i.e. biotin-labeled unlocker bound with streptavidin coated beads) was added in an approximately tenfold molar excess relative to the locker strands. The mixture was subsequently allowed to interact, i.e. the locker and passwords strand were allowed to interact, for about 1 hour under gentle mixing conditions to prevent sedimentation of the magnetic particles. After 1 hour the mixture was placed on a magnetic rack to separate the magnetic beads bound with unlocker and bound with unlocker complexed with locker, from the bulk solution. The supernatant obtained was subsequently PCR amplified using universal U_{FW} and U_{RV} primers. The resulting mixture was PCR purified and analysed using qPCR to determine relative concentrations of templates A and B. No significant differences were observed between unlocked samples and a control reference that did not contain locker strands, demonstrating successful unlocking. Highly advantageously, a statistically significant (p≥0.0016) difference of approximately 7 PCR cycles was observed (see FIG. 8) between samples that contained the locker strand and were unlocked with the corresponding unlocker, compared with a locker containing sample which was subjected to a non-complementary password strand, i.e. an incorrect unlocker. These results demonstrate that unlockers can be used to reliably remove locker strands from a mixture containing (double stranded) polynucleotide fragments and lockers, as described herein.

TABLE 5
obtained Ct values as depicted in FIG. 8

| | Template A | Template B | ΔCt |
|---|---|---|---|
| Beads with | 14.66 | 17.51 | −2.85 |
| password | 13.71 | 16.72 | −3.01 |
| | 19.02 | 19.77 | −0.75 |
| No locker | 13.9 | 15.55 | −1.65 |
| added | 14.06 | 17.11 | −3.05 |
| | 18.61 | 21.09 | −2.48 |
| Beads with | 19.28 | 12.53 | 6.75 |
| random | 20.03 | 16.26 | 3.77 |
| sequence | 19.47 | 15.11 | 4.36 |
| No beads | 19.73 | 13.88 | 5.85 |
| added | 19.88 | 15.74 | 4.14 |
| | 19.98 | 15.9 | 4.08 |

### Example 3

### Locking DNA-Encoded Data Using Molecular Encryption

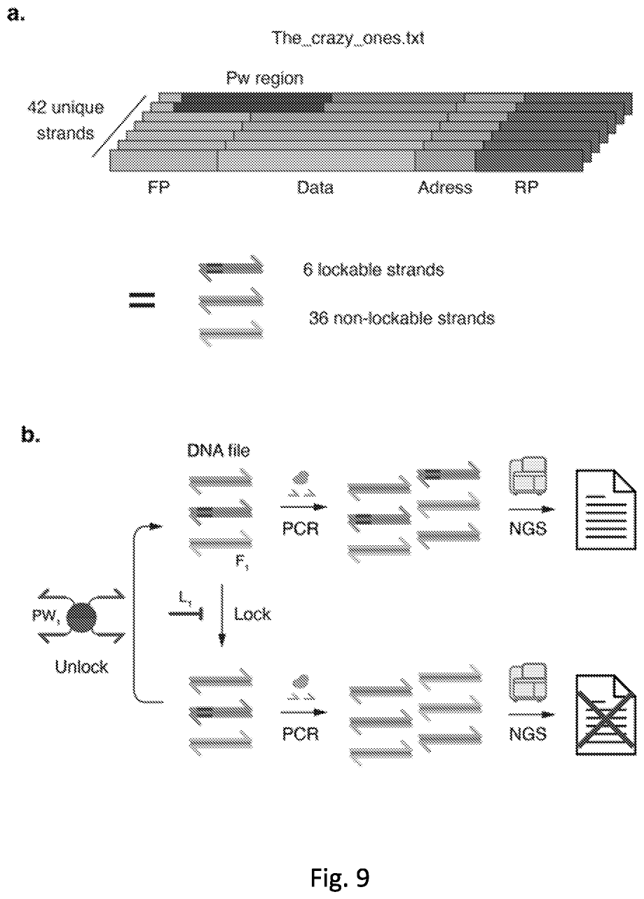

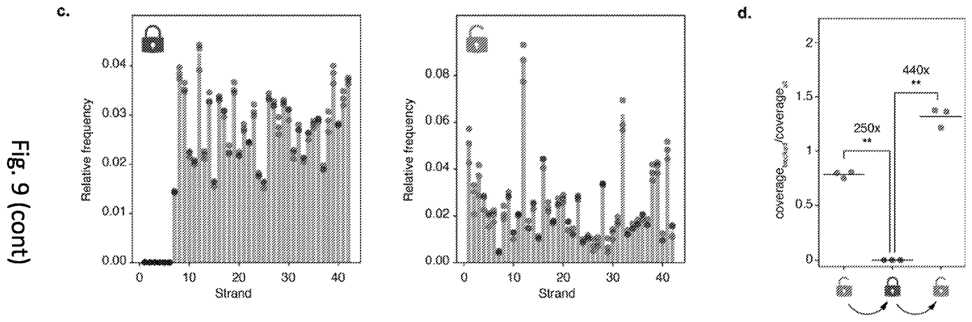

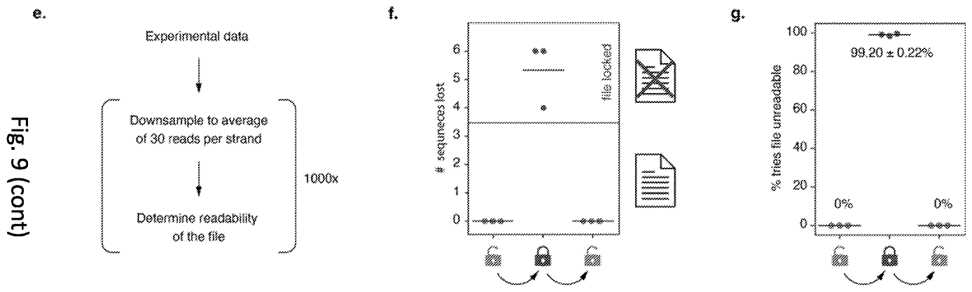

Having demonstrated that locker strands can introduce a meaningful and controllable PCR amplification bias into the two-template system (as described in e.g. Example 1 and shown in e.g. FIG. 2), we next applied it to DNA-encoded data. To this end, a data encoder/decoder was developed to encode digital data in DNA to allow data to be encrypted using locker 1 (FIG. 9b; L₁). As a proof of concept, a 549-byte text file (i.e. File 1) was encoded onto 42 DNA (SEQ ID NO: 23-64; Table 7) sequences representing the nucleic acid-encoded dataset (FIG. 9a). Of these 42 DNA sequences of the nucleic acid-encoded dataset six sequences were labelled with a locker target sequence (PW region) complementary to the locker to prevent their amplification in the presence of the locker. For Example 3, the same sequences as used in Example 1 of the locker target sequence, locker and unlocker, as well as the primers were used as these as these were deemed effective (Tables 1 and 3). The designed nucleic acid-encoded dataset was synthesized to provide for 42 polynucleotide fragments representing the nucleic acid-encoded dataset. (FIG. 9a). In the locked state, PCR amplification would then be biased toward non-labelled strands, i.e. strands lacking a locker sequence, rendering a critical number of segments unreadable for use in DNA decoding. Sequestration of the locker strands using molecular passwords, i.e. unlockers, subsequently restores data access. This encoding and selective data access workflow is referred to as DNA Gated Unlocking and Access Restriction of Data (DNA-GUARD) below and is in accordance with the invention.

The impact of locking and unlocking on PCR amplification bias using Next Generation Sequencing (NGS; FIG. 9b) was quantified (for details on library preparation and sequencing see Methods below). Sequencing results (FIG. 9c) showed the selective suppression of lockable strands in the locked state, which was restored by unlocking File 1 using superparamagnetic particle-bound molecular passwords, i.e. unlockers (hereafter simply molecular passwords, i.e. PW1 in FIG. 9b). In the absence of bias, a uniform distribution of reads was expected. Sequencing the PCR products of locked File 1 shows (FIG. 9c left graph) severely repressed amplification of the 6 strands containing the PW region, i.e. locker target sequence, indicating that biassing of PCR amplification also works for data-encoding DNA. Unlocking of File 1 by molecular passwords, i.e. unlockers, negates PCR amplification bias, as demonstrated by the equal sequencing coverage across all strands (FIG. 9c right graph), indicating successful restoration of PCR fidelity. Amplification uniformity was also expressed as the ratio of relative reads for the 6 lockable and the 36 non-lockable strands (FIG. 9d). Before locking File 1, a uniform sequencing coverage was observed, as demonstrated by the fact that the ratio between reads for lockable and non-lockable strands was 0.722±0.054 (mean±standard deviation). After locking, this ratio is changed approximately 250-fold to 0.00287±0.00014 (mean±standard deviation), indicative of severely biased PCR amplification. Following unlocking, the ratio is 1.26±0.16 (mean±standard deviation), indicating even amplification and sequencing coverage.

Having demonstrated that DNA-GUARD can be used to controllably bias the PCR amplification of DNA-encoded data, the performance of DNA-GUARD was further investigated to prevent unwanted data access, i.e. decoding of locked sequences. Although the sequencing coverage was sufficiently high to detect a small number of reads for the lockable strands, meaning files were readable under all conditions, typical coverages in DNA data storage are much lower (References 1 and 2) Therefore, realistic DNA data storage conditions were simulated (FIG. 9e) by down sampling sequencing data to a 30× coverage, meaning 30 reads per strand on average (See Methods: Analysis of Illumina sequencing data). It was evaluated whether at least 4 of the 6 lockable strands had one or more reads, which would enable file decoding. It was confirmed that the method worked for three independent experiments, down sampled once by plotting the number of no longer readable sequences at a 30× coverage (FIG. 9f) and determined File 1 could not be decoded in the locked state. At the same time, it was readable in both unlocked states. Repeating this process 1000 times, it was found that in 99.2% of simulations, File 1 could not be decoded in the locked state (FIG. 9g). These results demonstrate that DNA-GUARD effectively encrypts and decrypts DNA-encoded digital data, allowing precise control over data accessibility.

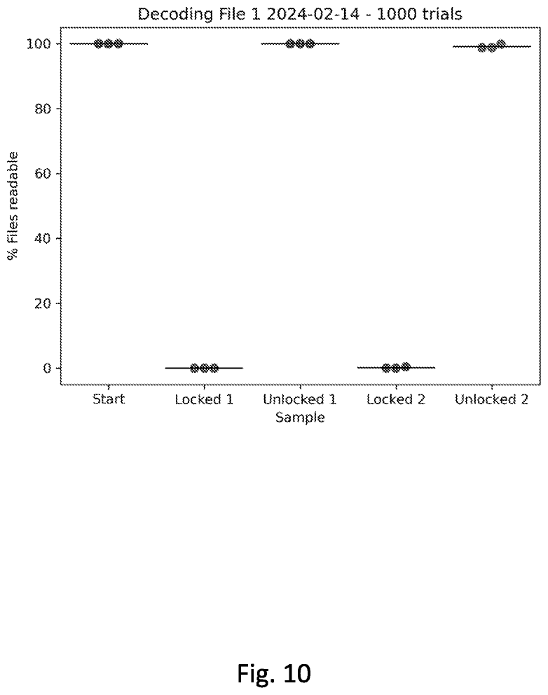

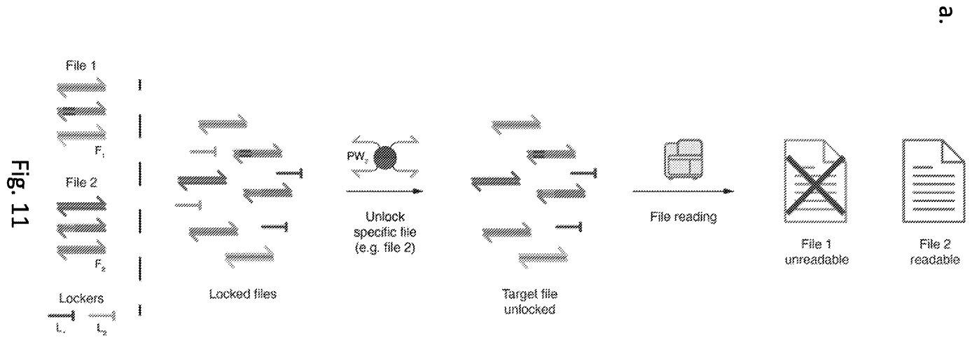

FIG. 10 shows that the 549-byte text file of Example 3 was unreadable in the locked state 100% of the trials (0% of the files readable), while it was readable 100% of the trials after the first unlocking and 99.2% of the trials after the second unlocking

This Example shows that using physically molecularly encrypted nucleic acid-encoded data, and methods, as described herein, for physical molecular encryption of nucleic acid-encoded data allows for repeatable locking and unlocking of nucleic acid-encoded data. Highly advantageously, the nucleic acid-encoded data can be accessed multiple times by one or more users over any period of time, akin to traditional methods of storing and encrypting data, for example using a flash drive comprising encrypted data

### Methods

### Materials

All chemicals and magnetic particles used were purchased from Sigma, unless otherwise noted. Enzymes were purchased from New England Biolabs, unless otherwise noted. Reagents used for Next Generation Sequencing and library preparation were ordered from Illumnia. EvaGreen was ordered from Biotium, and KAPA HiFi HotStart PCR Kit was ordered from Roche.

### DNA Oligonucleotide Synthesis

DNA oligonucleotides were synthesized as described in Example 1.

### PCR

PCR reactions were performed as described in Example 1.

### Magnetic Removal of Locker Strands

Molecular passwords were prepared by first washing and resuspending 200 μL Streptavidin-coated DynaBeads according to manufacturer's instruction for conjugating biotin-labelled DNA. To the washed and resuspended beads 400 μL of 5 μM biotin-labelled DNA was added. The resulting mixture was incubated under gentle agitation for 60 minutes at room temperature. After the DNA was conjugated excess unbound DNA was removed according to manufacturer's instruction. Finally, the beads were resuspended in a final volume of 10 μL to yield the final molecular password solution.

In a typical unlocking experiment, 10 μL molecular passwords was added to 200 μL of locked file solutions. The resulting mixture was incubated for 60 minutes at room temperature under gentle agitation. After incubation, the mixture was placed on a magnetic separation rack for 3 minutes to separate out the beads, and the supernatant was pipetted to yield an unlocked file solution.

qPCR

Quantitative PCR was performed as described in Example 1.

### Library Preparation and Sequencing

Samples were prepared for sequencing following the Illumina TruSeq Nano DNA Library Prep protocol. Ends were blunted with the End-Repair buffer (ERP2), then purified with Beckman Coulter AMPure XP beads, and an ‘A’ nucleotide was annealed to the 3′ end with A-tailing Ligase (ATL). Ligation was performed using Illumina sequencing adapters from Illumina's TruSeq DNA CD Indexes kit, with each sample ligated to a unique Illumina index. Finally, the samples were cleaned using Illumina Samples Purification Beads (SPB) and enriched using a 12-cycle PCR. Final product length and purity were qualified using a QIAxcel Bioanalyzer. Samples were then individually quantified using qPCR and mixed to create an equal-mass library.

A final library was prepared for sequencing by following the Illumina NextSeq Denature and Dilute Libraries Guide. Sequencing libraries were loaded in the Illumina NextSeq at 1.3 μM with a 20% control spike-in of ligated PhiX genome.

### Analysis of Illumina Sequencing Data

Basecalling and demultiplexing of sequenced samples was performed using bcl2fastq. The generated FASTQ-files were then aligned against reference sequences using custom Python code. As a first step, sequences that didn't meet quality or length requirements were discarded before proceeding with analysis. We randomly sampled sequences from the total passable sequences until the desired number of sequences were drawn, for example to an average coverage of 30 (typically expressed as desired coverages times the number of sequences in a file).

To generate the distribution of reads across files, the sampled reads were subsequently aligned to the reference sequences using Burrows-Wheeler Aligner before the coverage for each sequence was determined using SAMtools.

To determine file readability, the sampled reads were passed to the decoding pipeline. This, briefly, consisted of clustering reads before extracting the N highest scoring clusters, where N is the number of strands encoding a file. Clusters were then passed to the decoding algorithm and decoding was attempted and reported as either successful or unsuccessful.

### Statistics

All results reporting statistical values were from independent triplicates. Analysis was performed using Python's SciPy (Python 3.6.5, SciPy version 1.1.0) library. Welch's two-sided t-test was used to compare two populations. Statistical significance between more than two samples was determined using One-way Analysis of Variance (ANOVA) followed by post-hoc analysis, using Tukey's multiple comparison testing. Only values of p<0.05 were considered statistically significant.

### Example 4

### Scalability of the System Using Multiple Nucleic Acid-Encoded Datasets

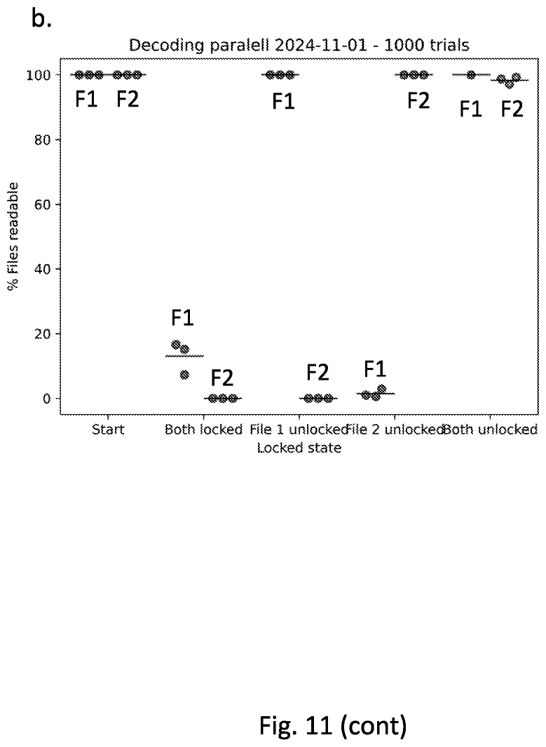

Example 3 shows that it was possible to encrypt a single text file. A similar concept as used Example 3 was now applied in a system wherein multiple text files were locked (and unlocked) in parallel, i.e comprised in the same system. FIG. 11a shows a schematic outline of the approach used here, wherein a specific file (e.g. File 2) is unlocked using a specific locker (L₂) such that it can be read (decoded). As in classical encryption systems it was shown that the nucleic acid-encoded datasets were individually lockable and unlockable. Using the same encoding scheme as described in Example 3 for the first text file (FIG. 11b; F1 and Table 7), a secondary 606-byte text file was encoded into a nucleic acid-encoded datasets using a second locker target sequence and locker set (FIG. 11b; F2. Table 6 for sequenced for target sequence and locker set). Hence, a second set of 46 DNA sequences (SEQ ID NO: 65-110; Table 8) of the nucleic acid-encoded dataset six sequences were labelled with a second locker target sequence complementary to the second locker to prevent their amplification in the presence of the second locker. In accordance with the invention, the designed second nucleic acid-encoded dataset was synthesized to provide for a second set of 46 polynucleotide fragments representing the second nucleic acid-encoded dataset.

After mixing the two sets of DNA-encoded files, each individual DNA-encoded file or both DNA-encoded file was attempted to be decoded simultaneously before locking and subsequently sequencing the PCR products. As described for Example 3, decoding was attempted 1000 times to estimate the probability of successfully decoding the files. The second text file displayed the anticipated results wherein in the locked state 100% of the trials the second text file was unreadable (0% of the files readable). It was further observed that the first text file could be successfully decoded in the unlocked states. The first text file could not be read in most of the trials (in 87% of the trials when both first and second text files were locked; and in 98.5% of the trials when only text file 2 was unlocked). These results show the abilities of the system to allow for parallel file encryption and decryption of multiple nucleic acid-encoded datasets all comprised in one.

The skilled person is able, by using i.a. the approach disclosed herein above, to design and test nucleic acid-encoded datasets, and combinations thereof, to thereby determine combinations of lockers and unlockers that provide the desired accessibility, e.g. the desired percentage of readability at a defined coverage or at defined number of reads. Hence, highly advantageously, by applying the means and methods as described herein, mixed combinations of datasets can be generated, each of the datasets being only accessible through the use of the unlocker, preferably wherein the unlocker is specific for each dataset.

### Example 5

### Improved Efficiency of a Unlocker (PW) Using an Inverted dT (InvdT)

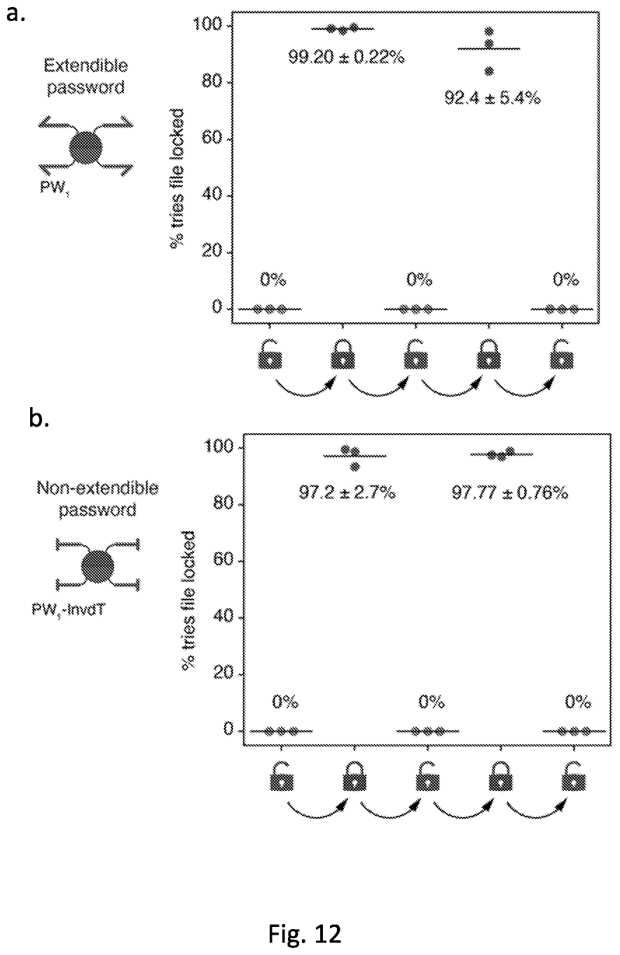

FIG. 12a and FIG. 12b demonstrate a different unlocker design wherein the unlocker is non-extensible to ensure any unbound unlocker cannot (or is less likely to) serve as a primer in a PCR reaction. Unwanted binding to an unbound unlocker reduces the effectiveness of the locking strategy. Hence, in some embodiments in accordance with the invention, the unlocker comprises an inverted dT to prevent unwanted PCR amplification.

TABLE 6
DNA sequences of primers (FW/RV), lockers (L) and
unlockers (P) used in Example 4

| | SEQ- | | Length | |
|---|---|---|---|---|
| Name | ID | Sequence | (#nt) | Modification |
| FW1 | 9 | TGTCTCACTAGGTCCTCTCA | 20 | |
| RV1 | 10 | GCGTCGAAACTGAAACTCCT | 20 | |
| FW2 | 19 | TGAGTACCGCTTCACACAGA | 20 | |
| RV2 | 20 | CGGAGGCATTGAGTACGATG | 20 | |
| L1 | 15 | GCTCTTCCCACCACCATTGCCTTCT | 28 | 3′ inverted dT |
| | | TCT | | |
| L2 | 21 | GCTCTTCCCACCACCATTGCCTTCT | 30 | 3′ inverted dT |
| | | TCTTG | | |
| P1 | 18 | AAGAAGAAGGCAATGGTGGTGGGA | 44 | 5′ biotin |
| | | AGAGCTGAGAGGACCTAGTG | | |
| P2 | 22 | CAAGAAGAAGGCAATGGTGGTGGG | 30 | 5′ biotin |
| | | AAGAGC | | |

TABLE 7
DNA sequences (42 total) encoding File 1

| Seq | SEQ-ID | Sequence |
|---|---|---|
| F1-1 | 23 | TGTCTCACTAGGTCCTCTCAGCTCTTCCCACCACCATTGCCT |
| | | TCTTCTTTACTGCTAGAACGTGCGTCTAGCATACACAGATCA |
| | | CGCGTGCAAGACTCATCGTAATCTACCTGAGGAGTTTCAGTT |
| | | TCGACGC |
| F1-2 | 24 | TGTCTCACTAGGTCCTCTCAGCTCTTCCCACCACCATTGCCT |
| | | TCTTCTTTAGTCTGTGAGTCATGAGATCACTCGAGTGCACTG |
| | | TGCGTTGCTACCATTCACAGACGTAGCTGAGGAGTTTCAGTT |
| | | TCGACGC |
| F1-3 | 25 | TGTCTCACTAGGTCCTCTCAGCTCTTCCCACCACCATTGCCT |
| | | TCTTCTTTCTCGACTGCGTCGCAGATTCGACGGAGTCTCAG |
| | | CGTAGTGCTCTACTCAGAGTCTCAGTATCGAGGAGTTTCAGT |
| | | TTCGACGC |
| F1-4 | 26 | TGTCTCACTAGGTCCTCTCAGCTCTTCCCACCACCATTGCCT |
| | | TCTTCTTTTGCCAGATCGTCGCATCATCACATACGCAGTAGA |
| | | TCCGTAGATGATAGTCTACGTCGTCGCATAGGAGTTTCAGTT |
| | | TCGACGC |
| F1-5 | 27 | TGTCTCACTAGGTCCTCTCAGCTCTTCCCACCACCATTGCCT |
| | | TCTTCTTTGAGTGCACTCACGCAGTCTAGAGCGCTTCTAGCA |
| | | CACGATGCTAGCATCAGTGAACAAGACTAAGGAGTTTCAGTT |
| | | TCGACGC |
| F1-6 | 28 | TGTCTCACTAGGTCCTCTCAGCTCTTCCCACCACCATTGCCT |
| | | TCTTCTTTGCATCTCAGCGTAGTGCTCTACTCAGACAGATCG |
| | | TCATGAGATGAACGGACTGCGCTTCGAGCAGGAGTTTCAGT |
| | | TTCGACGC |
| F1-7 | 29 | TGTCTCACTAGGTCCTCTCAGCTCTCCTACAGAGCCGAGCA |
| | | CACTCGAGAATGCTGCTGACACGTTGCTGATAGTCTTGCAC |
| | | TCACGCAGTCTAGAGCGCTATCCACCTGAGTAGGAGTTTCA |
| | | GTTTCGACGC |
| F1-8 | 30 | TGTCTCACTAGGTCCTCTCAAGTCTCACACTGTGTTCACGAT |
| | | GATAGACTGCTTCTACTACACGATCTTGATAGGCTCTGGAGC |
| | | GTAGTGCTTGTCTCAGATCTTCGAGCCGAAGGAGTTTCAGTT |
| | | TCGACGC |
| F1-9 | 31 | TGTCTCACTAGGTCCTCTCAAGTCATGAGTGCACTTAGCGTA |
| | | GTTGTCATACTTCTAGCACAACAGCTTCACTCGACCTGGTGA |
| | | CACGTGCACATGTGGCTGTGCACCTGTCTAGGAGTTTCAGT |
| | | TTCGACGC |
| F1-10 | 32 | TGTCTCACTAGGTCCTCTCAAGTACAATCCTGACTACAACAG |
| | | CTTCGTCGTCTCAGCTATAGACAGCTTGTTCGAGACTGTGA |
| | | GTCATGGCATAGCATGAGATCTCGCAGCTAAGGAGTTTCAG |
| | | TTTCGACGC |
| F1-11 | 33 | TGTCTCACTAGGTCCTCTCAAGTTAGGCTTCTGTGATCGCAA |
| | | CATCGACGATGTCTCAGGTCGCAAGATCACATGCACAGTAG |
| | | ACACGTTGCTAGCATTCTTCATGTGACAGCAGGAGTTTCAGT |
| | | TTCGACGC |
| F1-12 | 34 | TGTCTCACTAGGTCCTCTCACTCCTCATCCTGGTGTGAACAT |
| | | GAAGAAGTATCCTGACTACACGTACAAGCTCGACACAGATC |
| | | GTCGCATGATCGTCGATCTGTATGTGCTAGAGGAGTTTCAG |
| | | TTTCGACGC |
| F1-13 | 35 | TGTCTCACTAGGTCCTCTCACTCAGCGCTCAGTAGCGTCGA |
| | | TGATAGACTGCTCTGTGTACGAGTGCTTGAACGCTACTGGT |
| | | GACACGTGCACATGTGGCTAGAGCTCGAGATAGGAGTTTCA |
| | | GTTTCGACGC |
| F1-14 | 36 | TGTCTCACTAGGTCCTCTCACTCACAACTCTGTAGACAAGTG |
| | | CTTGTCTCAGACAGCTATGAACAGCTTCACTCACTTCTTAGA |
| | | CAACAGCTTCGACGGACATCCAGGATGAGAGGAGTTTCAGT |
| | | TTCGACGC |
| F1-15 | 37 | TGTCTCACTAGGTCCTCTCACTCTAGGCTTCTAGCACACGTG |
| | | CATGACTCGAGCAGCTGTCAAGTGCTTAGACGGACTCTAGA |
| | | GTCATGAGATCACTCGAGCGTGCTCTCTACAGGAGTTTCAG |
| | | TTTCGACGC |
| F1-16 | 38 | TGTCTCACTAGGTCCTCTCATGCACTGCTTCTCTGTCAGCAA |
| | | GTTGTCTCCTATCTCAGGTCATGAGTTGTCATGACGAGTACG |
| | | TCGAGACATCGTCGCTAGCTCATATCCGAAGGAGTTTCAGTT |
| | | TCGACGC |
| F1-17 | 39 | TGTCTCACTAGGTCCTCTCATGCAGCGCTCAGCTGGTCCGA |
| | | TGAAGACTCGTGTCTCTACGTCGTAGATAGAGCGCTTCTGT |
| | | GATCGCACACTCGAGCCAGTGAACTCTGTCAAGGAGTTTCA |
| | | GTTTCGACGC |
| F1-18 | 40 | TGTCTCACTAGGTCCTCTCATGCGTGGCTCAGGTGATCCGT |
| | | GCATACCATATGTCTAGCACACGACACAGACTCCGTCTGGA |
| | | GTCAGCAGTCAGACTCATCACTACGGTATGAAGGAGTTTCA |
| | | GTTTCGACGC |
| F1-19 | 41 | TGTCTCACTAGGTCCTCTCATGCTCGAGACAGCTACGAACAT |
| | | ACAGACTCATGCTGGTGCGTCGTTGCTCACATGCAGACTGC |
| | | GTCGCAGATTGAGTGGCTGCTCTCCACATGAGGAGTTTCAG |
| | | TTTCGACGC |
| F1-20 | 42 | TGTCTCACTAGGTCCTCTCAGAGCTCCGACTGGAGCGAGCA |
| | | ACATAGACGTAGTGCACTTCAGCAGTCTAGCATCTGGAGTA |
| | | CGTCTGCAGTTACACGGACATCCTAGCTGATAGGAGTTTCA |
| | | GTTTCGACGC |
| F1-21 | 43 | TGTCTCACTAGGTCCTCTCAGAGCATCTATCTGTCGTCATGA |
| | | GATCACTCGAGTGCACTCGTCGATGATCGCTCTAGTGCACT |
| | | TCAGCAGTCTCACATTCTCAGCGTCGTTCGAGGAGTTTCAGT |
| | | TTCGACGC |
| F1-22 | 44 | TGTCTCACTAGGTCCTCTCAGAGACGATGTGCACTTGACGA |
| | | GATTGTAGCGCTCAGCTGGTCCGATGAAGCTCGATCTGCAC |
| | | TACAGCAGATAGACTCAGCTACTCGGCTATGAGGAGTTTCA |
| | | GTTTCGACGC |
| F1-23 | 45 | TGTCTCACTAGGTCCTCTCAGAGTCGACGTGCACTATCCGA |
| | | TCTTCGACGGACTCTAGCACAACAGCTTGTCTCAGACAGCT |
| | | ACGAACATGAAGAAGTAGTGTGCAGTACTGCAGGAGTTTCA |
| | | GTTTCGACGC |
| F1-24 | 46 | TGTCTCACTAGGTCCTCTCAGCACTCGAGCAGCTGGTCCGT |
| | | CACTGATCGGAGTGCACTTCAGCAGTCTAGCATTAGTGCAC |
| | | TGTGCGAGTCTACCATTCTACTATGCGACATAGGAGTTTCAG |
| | | TTTCGACGC |
| F1-25 | 47 | TGTCTCACTAGGTCCTCTCAGCACATATGCAGATCGTCATGA |
| | | GATCACTCAGCCTGTAGACGCGTGCACATGTGGCTCACGTG |
| | | ATCGCACACGATAGCGCTAGTAGTCGTGACAGGAGTTTCAG |
| | | TTTCGACGC |
| F1-26 | 48 | TGTCTCACTAGGTCCTCTCAGCAACGGTCTCTCTATAGCGA |
| | | GTCAGACTCATCCTGACTACAACAGCTTCACTCCTACTGCTA |
| | | GTCCGATGAAGACTCACATCTTCGAGAGCTAGGAGTTTCAG |
| | | TTTCGACGC |
| F1-27 | 49 | TGTCTCACTAGGTCCTCTCAGCATCGACTCAGCTGACAACA |
| | | GCTTAGACGGACTCTAGCTCGCGAAGTTGAACGTGCGAGTA |
| | | CGTCTGCAGTTCGACGTGCACTGTCGTCACTAGGAGTTTCA |
| | | GTTTCGACGC |
| F1-28 | 50 | TGTCTCACTAGGTCCTCTCAATGACTGCTTCTTGTATCGCAA |
| | | CATCGCTCGAGTGCACTTAGCGAGATTCGCATGAGTGCACT |
| | | CGACGAAGTGATAGCGCTGCAGAGCGTGTAAGGAGTTTCAG |
| | | TTTCGACGC |
| F1-29 | 51 | TGTCTCACTAGGTCCTCTCAATGCATACGCAGCTAACAACAG |
| | | CTTGTCTCAGACAGCTACGAACAGCTTACCATACGTGCACTT |
| | | CAGCAGTCTAGAGCGCTCTCACATCATGTAGGAGTTTCAGTT |
| | | TCGACGC |
| F1-30 | 52 | TGTCTCACTAGGTCCTCTCAATGACGCTCTCTAGCGTCCGTA |
| | | TCGATAGCGCTCTGTGTCGTGCACACTGATAGCAGTGCACT |
| | | TCGCGACACAGACTCACGAGCTACGTAAGTAGGAGTTTCAG |
| | | TTTCGACGC |
| F1-31 | 53 | TGTCTCACTAGGTCCTCTCAATGTCGGAGCAGATCGTCGCA |
| | | TCTTAGCATTCTCTGGAGTCACGTGCACATGTGGCTAGAAG |
| | | CACACGAGCATACCATCTAGATGTAAGTGAGAGGAGTTTCA |
| | | GTTTCGACGC |
| F1-32 | 54 | TGTCTCACTAGGTCCTCTCAAGACTCACGCAGATCGTCATGA |
| | | GATCACTCGAGTGCACTTACGCACACTCGTCGGTCCTGGTG |
| | | ACAACAGCTTGTTCGAGAGTGGAGGATCTCAGGAGTTTCAG |
| | | TTTCGACGC |
| F1-33 | 55 | TGTCTCACTAGGTCCTCTCAAGACATGACTGCACTGTCCGTT |
| | | GCTAGAGCGCTCAGCTGTAGGCAAGTGATACGTAGTGCACT |
| | | ACACGATGATCGTCGCTAACGTGCCTGCATAGGAGTTTCAG |
| | | TTTCGACGC |
| F1-34 | 56 | TGTCTCACTAGGTCCTCTCAAGAACGATGCTGGCTGTCATG |
| | | AGATCGTAGGCTTCTGTGATCGCAACATCGACGCATTGCAC |
| | | TTCAGCAGTCTAGCATTAGGTAACGTCACGAAGGAGTTTCA |
| | | GTTTCGACGC |
| F1-35 | 57 | TGTCTCACTAGGTCCTCTCAAGATAGGCTCAGCTGGTCCGA |
| | | TGAAGACTCCTCCTGACTGTCCGATGATAGTCGGAGTGCAC |
| | | TTCAGCAGTCTAGAGCGCTCATAGAATGACGAGGAGTTTCA |
| | | GTTTCGACGC |
| F1-36 | 58 | TGTCTCACTAGGTCCTCTCAAGCCTCCGTCTGTGTTAGGCAT |
| | | ACTAGACTCAGTGCACTGTCCGTTGCTAGAGCGCTTCTGTG |
| | | ATCGCACACAGACTCGACGTCACGTCTATGAGGAGTTTCAG |
| | | TTTCGACGC |
| F1-37 | 59 | TGTCTCACTAGGTCCTCTCAAGCCATTCTCAGCTATAGACAG |
| | | CTTGTTCGAGACTGTGAGTCGCAAGATCGTAGTCTTGCGCT |
| | | GCTGCTGCTGCTGCTGCTAGCGCATGCCTCAGGAGTTTCAG |
| | | TTTCGACGC |
| F1-38 | 60 | TGTCTCACTAGGTCCTCTCAAGCGTCTGACTGACAGTGTAGT |
| | | CAGATGTGAGTGTAGTGACTGAGAGACTAGAGGATGATTCA |
| | | CGAACAACTTCTTGCTCTTCAGCTACTATCAGGAGTTTCAGT |
| | | TTCGACGC |
| F1-39 | 61 | TGTCTCACTAGGTCCTCTCAAGCTACGTAAGCAGAATGCACT |
| | | GACTAGATCTCACAGACTCTCAGGTGGCTTACCTGCTCCGA |
| | | GTAACAGACTCGTAGGCTACGTCGACTGAGAGGAGTTTCAG |
| | | TTTCGACGC |
| F1-40 | 62 | TGTCTCACTAGGTCCTCTCACACATGTCGACTTGTCTGCGAG |
| | | ACTGCCAGCACCTAAGATCGTAGGTATAGGAGGCAGATGTC |
| | | GAGACTCGAGCAGCAGAGGAGGTATACACGAGGAGTTTCA |
| | | GTTTCGACGC |
| F1-41 | 63 | TGTCTCACTAGGTCCTCTCACACCTGGATTAGAGTGATTGCG |
| | | TGATGAGCCGAAGTGATACTTCAGAGACACTCCACACTAGA |
| | | CTATGCTACTAGAGCTCAGAGAGATCGGAGAGGAGTTTCAG |
| | | TTTCGACGC |
| F1-42 | 64 | TGTCTCACTAGGTCCTCTCACACATCAGTACACTCAGTCTAA |
| | | TCTGATCGGACGTAGCTTAGGATCGTTCAGATTCTAGTGTGT |
| | | CTCATTCGATCTCTCACGTGTACAGCAGCAGGAGTTTCAGTT |
| | | TCGACGC |

TABLE 8
DNA sequences (46 total) encoding File 2

| Seq | SEQ-ID | Sequence |
|---|---|---|
| F2-1 | 65 | CGGAGGCATTGAGTACGATGGCAGTGGTGGTGGATAGACT |
| | | CGTAGATGTCCGTTGCGTCTGCACTTGCAGACATTACCAGC |
| | | GTACAACTTGCGAGCTCGCTTACGACAGAGAGTCTGTGTGA |
| | | AGCGGTACTCA |
| F2-2 | 66 | CGGAGGCATTGAGTACGATGGCAGTGGTGGTGGATAGACT |
| | | CGTAGATTACTGAAGACGTATCCTACTGACTCTCTGTCACGC |
| | | AACAACTTGCAGACATGCTCAGCATACACGTTCTGTGTGAAG |
| | | CGGTACTCA |
| F2-3 | 67 | CGGAGGCATTGAGTACGATGGCAGTGGTGGTGGATAGACT |
| | | CGTAGATTACTCGTCTATGGTCATCCTGACTACGTCGACAAT |
| | | GGTCGTCCTGGAGACGGCTATCGTCATGTGCTCTGTGTGAA |
| | | GCGGTACTCA |
| F2-4 | 68 | CGGAGGCATTGAGTACGATGGCAGTGGTGGTGGATAGACT |
| | | CGTAGATGCTTGTAGACGTACAAGCTCTACGCTCAGAAGAA |
| | | TGATCCTACAGACATCGGCTGCTTCTGACGCATCTGTGTGA |
| | | AGCGGTACTCA |
| F2-5 | 69 | CGGAGGCATTGAGTACGATGGCAGTGGTGGTGGATAGACT |
| | | CGTAGATAGATAGGCTACAGTCAGCTCTGAGCTCAGAACAG |
| | | CACGTACTTGCATCCTCACTAGCGAGTGTGTATCTGTGTGAA |
| | | GCGGTACTCA |
| F2-6 | 70 | CGGAGGCATTGAGTACGATGGCAGTGGTGGTGGATAGACT |
| | | CGTAGATGCTTCACACGCAGTCATCAGATCTTCGTACACAC |
| | | GTGTGGTGCTGGACCATACTCATTCAAGAAGATCTGTGTGA |
| | | AGCGGTACTCA |
| F2-7 | 71 | CGGAGGCATTGAGTACGATGGTCCATTACGCGGCAACAGAG |
| | | CTGACTCTCCATGCTACAGTCTAGCTGTGCCTCAGAACATG |
| | | CGTCGCTCTGACTACGACTTACTGCCGATCATCTGTGTGAA |
| | | GCGGTACTCA |
| F2-8 | 72 | CGGAGGCATTGAGTACGATGCGACATTAGGCTACAGTCCTG |
| | | TCTACGCATTGTTCACGAACAGTGCAGGCTACTTGTGTCCG |
| | | AGTCGTGTCTGCTTAGACTCTGAGAACGTGATCTGTGTGAA |
| | | GCGGTACTCA |
| F2-9 | 73 | CGGAGGCATTGAGTACGATGGAGCATTACGCAGCAATCACT |
| | | TGCGACACGTAGGCTACACGAGAGGACGCTGTGCAGAGAC |
| | | TCGTCATCCTGAGCCTCAGTCACGTGCGTTGCTCTGTGTGA |
| | | AGCGGTACTCA |
| F2-10 | 74 | CGGAGGCATTGAGTACGATGCAGCTCTCGACACGACGTTAG |
| | | CTGGCTGTGTACGATCGACGTCTGCTGACGCTCAGAACACG |
| | | ATAGACTTGCACTCATAGTTCGCGAATCTACTCTGTGTGAAG |
| | | CGGTACTCA |
| F2-11 | 75 | CGGAGGCATTGAGTACGATGACGCTCAGATGCCGAATCTGT |
| | | CAGGCTAGCTACCGCATGGTCTGCCAGTCTCTCAGACACTG |
| | | CCGTCTGCAGTAGACGAGTTCTATCAGCCAGTCTGTGTGAA |
| | | GCGGTACTCA |
| F2-12 | 76 | CGGAGGCATTGAGTACGATGCTCCTCTCGGATGCATACGAG |
| | | CAGGAGCTCTAGACACGAGTCACTTGCCGATCGTCGTACCG |
| | | ATCACTACTGGAGTCGAGTTCGGTCTGCATCTCTGTGTGAA |
| | | GCGGTACTCA |
| F2-13 | 77 | CGGAGGCATTGAGTACGATGCAGACTAGAAGTGCACGTGTG |
| | | CAGGCTACTTCAAGTGCATCGCTACAGGCTTAGTACAGTGC |
| | | ACGACTGCAGCTACTCCTCATCTCATGCATGTCTGTGTGAAG |
| | | CGGTACTCA |
| F2-14 | 78 | CGGAGGCATTGAGTACGATGCAGCATTACAGACGTACAGTG |
| | | CAGCAGACTAGAAGAATGATCCTACAGTCTCTCCATGCTAGT |
| | | ATCTGTCTGCGTCATCTCGTACAGCAGTCTTCTGTGTGAAGC |
| | | GGTACTCA |
| F2-15 | 79 | CGGAGGCATTGAGTACGATGGCTAGCTCGAGTGCACGTGA |
| | | GGACGCTGTGTACACAGCATCACTGTCTGCTTAGTCGCAGG |
| | | CAGTCTGCCTGTCTACGCTCTGCAGCCTGTACTCTGTGTGA |
| | | AGCGGTACTCA |
| F2-16 | 80 | CGGAGGCATTGAGTACGATGGCATCGTCGTGCCGTCGAGA |
| | | GCAGATCCATTCAGATGCACGTACTTGCATCCTCTCACACG |
| | | CATAGCTACAGGCTTAGCTCCATGTAACGGTATCTGTGTGAA |
| | | GCGGTACTCA |
| F2-17 | 81 | CGGAGGCATTGAGTACGATGACTACGTGACACGCAGTCGCT |
| | | GAGGTGACTCAGTGACTATACATCGCAAGCGTGCATGCTAG |
| | | TCTCTGTCTGACACTCTGCCGTTCAGTATCTTCTGTGTGAAG |
| | | CGGTACTCA |
| F2-18 | 82 | CGGAGGCATTGAGTACGATGGCTAGCTAGAGTCGAGTGACT |
| | | CTGGCTGTGTACACAGCATCAACTTGCAGCAGTAGAAGTGC |
| | | ACGAGTGCTGGACTCCTGCGACTGCATGTCGTCTGTGTGAA |
| | | GCGGTACTCA |
| F2-19 | 83 | CGGAGGCATTGAGTACGATGCGTCTCAGAAGTCTCTACGCT |
| | | GCAGCTAGCTACAGACGTCGTCTACTGACGACGCATGCTAC |
| | | AGCTTAGCTGTGCACGTGCTAGCACTGCTGCTCTGTGTGAA |
| | | GCGGTACTCA |
| F2-20 | 84 | CGGAGGCATTGAGTACGATGGCTAGCTCAAGAATGGTCGTC |
| | | TCTCTAACGTGATGACGATAGACTTGCGACTCCTGTAGAATG |
| | | GTCGTCTCTAGATCGTGCTCTGTAGTAACTTCTGTGTGAAGC |
| | | GGTACTCA |
| F2-21 | 85 | CGGAGGCATTGAGTACGATGACTCTCTGTGCTACAGTCGTG |
| | | CTGCAGCTCAGAGATGCAACGACTTGCATCCTCTCACACGC |
| | | AGTCTGCCTGTCTCTCGAGCATCACGTGGTGTCTGTGTGAA |
| | | GCGGTACTCA |
| F2-22 | 86 | CGGAGGCATTGAGTACGATGGCATCGTCGTGCCGTCGAGA |
| | | GCAGATCCATTCAGATGCACGTACTTGCATCCTCTCAAGTGC |
| | | ATCAACTTGCCTGCATGAGCAGTGAAGAATGTCTGTGTGAA |
| | | GCGGTACTCA |
| F2-23 | 87 | CGGAGGCATTGAGTACGATGACTACGTCGGCTAGTATCGAG |
| | | CAGACGCTCAGAGCACGAGTCAGCTCTGAGACGTAGCACAT |
| | | GCGAGTGCTGTAGACGGAGCAGTAGTGCATCTCTGTGTGAA |
| | | GCGGTACTCA |
| F2-24 | 88 | CGGAGGCATTGAGTACGATGGCTAGCTACGCACGAGTGCTA |
| | | TCTCTGCATTGTTACCGAGTCGTGTCTGAGCTCTAGGCTACA |
| | | ATCTAGCTGGCTTAGGAGCGTTCATGTACTTCTGTGTGAAGC |
| | | GGTACTCA |
| F2-25 | 89 | CGGAGGCATTGAGTACGATGACTCTCTCGTACGCAGTCGTC |
| | | TCTAGACATTAGGCTAGTGTGTGTCTGGACTCGTCACGCAT |
| | | GGTCTGCCTGTCTCTCGCATGACATTAGCGATCTGTGTGAA |
| | | GCGGTACTCA |
| F2-26 | 90 | CGGAGGCATTGAGTACGATGGCTACTTGTGTCCGAACAACT |
| | | TGCCGTTCGTCGTGCATGCGAGTGCAGGCTTAGTACAGTGC |
| | | ACGTACTTGCAGTCATGCATCGGATGCATCATCTGTGTGAA |
| | | GCGGTACTCA |
| F2-27 | 91 | CGGAGGCATTGAGTACGATGGAGCTCAGATCTATGTAGGAG |
| | | CTGATCCTCTGTCACGCACGTACTTGCAGCACGTCGGCTAG |
| | | TTCAACTCTGAGCACGGCACTACAGCTAGACTCTGTGTGAA |
| | | GCGGTACTCA |
| F2-28 | 92 | CGGAGGCATTGAGTACGATGACGCTCAGACAGCGACGTAG |
| | | CTCTCTGCTCAGAACAGCACGTACTTGCACTCTCAGAGCAC |
| | | GATCAAGCCAGGAGTCGGCATCTCATGCTAGTTCTGTGTGA |
| | | AGCGGTACTCA |
| F2-29 | 93 | CGGAGGCATTGAGTACGATGGCTTAGTCGCAGGCAGTCATC |
| | | CTGACTCTCTGTCACGCATAGGAGCTGACTCTCTCGGCTAC |
| | | ACGTTAGCTGGAGCTCATGGCTAGTTCGTACTCTGTGTGAA |
| | | GCGGTACTCA |
| F2-30 | 94 | CGGAGGCATTGAGTACGATGGCTACTTGTTCTCGTCGTCTA |
| | | GAGGCTACTTCACACATGCGTGTGCAGACACATTAGAGAGC |
| | | ATCAACTCTGGCTAGCATGGAGCTATCACACTCTGTGTGAA |
| | | GCGGTACTCA |
| F2-31 | 95 | CGGAGGCATTGAGTACGATGGACACGTAGGCTACAGTCTAG |
| | | CTGGCTAGCTCATGACGAGTGACTCTGGCTTAGTGTACAGC |
| | | AACACTACAGCTGGTGATGCTCCTCCGAGACTCTGTGTGAA |
| | | GCGGTACTCA |
| F2-32 | 96 | CGGAGGCATTGAGTACGATGCTGAGCCATGCTACATCGACT |
| | | CTGAGCTCGTACGTCGCAGTCTGCCTGACGCTCAGAAGAAT |
| | | GATCCTACAGGCTTAGATGTCGACAGCGCTATCTGTGTGAA |
| | | GCGGTACTCA |
| F2-33 | 97 | CGGAGGCATTGAGTACGATGAGTCATTACTGCCGTACACTG |
| | | TCTATCCTCAGAGCTCGTATCCTACAGCTCCATTAGGCTACA |
| | | CGTTAGCAGGCTACTAGACATTGCGAGGAGTCTGTGTGAAG |
| | | CGGTACTCA |
| F2-34 | 98 | CGGAGGCATTGAGTACGATGTGCCATTGTGCAATGTCAACT |
| | | TGCATCCTCTCAAGTGCATCAACTTGCCTCCATTACTGAGCA |
| | | GTCAGACAGGAGCATAGATCAATGGATACGTCTGTGTGAAG |
| | | CGGTACTCA |
| F2-35 | 99 | CGGAGGCATTGAGTACGATGGCTAGCTCGAGTGCAACACTA |
| | | CAGGCTTAGTCGCACATGTCAACTTGCAGTCATGATGCTAGT |
| | | TCAACTCTGGAGACGAGATGATAGACAATGTCTGTGTGAAG |
| | | CGGTACTCA |
| F2-36 | 100 | CGGAGGCATTGAGTACGATGGCTACTTCAGCGCGTCGAGA |
| | | GCAGTCTCTCAGACACCGATGACTACAGTCTACTAGAGCAC |
| | | TACGTACTTGCATCTCGAGAACAAGCTCTTGTTCTGTGTGAA |
| | | GCGGTACTCA |
| F2-37 | 101 | CGGAGGCATTGAGTACGATGAGACATTAGTGCCGTACAACT |
| | | TGCAGCTCGTGAGCTAGTTACGTGCTGGAGACGTCGAGACG |
| | | TTGAACTTGCGACCTCAGCTGTCGTACTCTATCTGTGTGAAG |
| | | CGGTACTCA |
| F2-38 | 102 | CGGAGGCATTGAGTACGATGGCACTCAGATGCCGTCGTTGT |
| | | CTGCTGCTCAGAAGTGCATCAACTTGCATCCTCTCACACGC |
| | | AGTCAGACAGGACCATAGCCTGGTAGCTAGATCTGTGTGAA |
| | | GCGGTACTCA |
| F2-39 | 103 | CGGAGGCATTGAGTACGATGATCCTCTGTGATCGACGAGAG |
| | | TGCGCTACTGTGGATGCACGTTGTTGCATCCTCAGAAGACG |
| | | TACAGTGCTGCAGACGAGCATGCGAGTCACATCTGTGTGAA |
| | | GCGGTACTCA |
| F2-40 | 104 | CGGAGGCATTGAGTACGATGGCTAGCTCGCACGCAGTCTG |
| | | CCAGAGTTCGTCGCACATGTCAACTTGCCTGCATTCAGCAG |
| | | CATAGTGTCTGGCATCGAGCTCACGATGATCGTCTGTGTGA |
| | | AGCGGTACTCA |
| F2-41 | 105 | CGGAGGCATTGAGTACGATGAGCCTCTCGTCACGAGTCGCT |
| | | CATGCAGTGGCAAGTGTGATCGTGTCGCTCCTCTCAGTGCT |
| | | GCATGTCATGCGAACTCACACTCTGACTATCTCTGTGTGAAG |
| | | CGGTACTCA |
| F2-42 | 106 | CGGAGGCATTGAGTACGATGTCGGAGCTGGTCGTATACACG |
| | | GAGAGCGCAATCATCTAGTCAGAGGCTAGCTGCTAGAGCGC |
| | | TGAGGTATCGGTGAGCCACTCGGTCTGAGATTCTGTGTGAA |
| | | GCGGTACTCA |
| F2-43 | 107 | CGGAGGCATTGAGTACGATGTCAGTCTGCCACCGTATCCTC |
| | | CTCGACGTATGACTATAGCACTAGTCACATGTGTCAGCTCTC |
| | | TACCTACTCAGAGTGCACGTGTAGACGTAGTCTGTGTGAAG |
| | | CGGTACTCA |
| F2-44 | 108 | CGGAGGCATTGAGTACGATGAGAGAGTCTAGCCGACGACTA |
| | | ACGACAAGCCTGTGCACTAGCGTCATCACTGTAGTGGAGCG |
| | | ATCAGAGGTATCGTCAGCGCTACATTCGTGCTCTGTGTGAA |
| | | GCGGTACTCA |
| F2-45 | 109 | CGGAGGCATTGAGTACGATGTGTGACGCGTAGACGTGCTAC |
| | | TGATCTTGTACTCACTGTTGTCTGACGCGAGCATGCGAGCA |
| | | CGAGGCTTCAACGACTCATCGAGACGCATCATCTGTGTGAA |
| | | GCGGTACTCA |
| F2-46 | 110 | CGGAGGCATTGAGTACGATGTGTATCTGCTCTCATAGTGAG |
| | | AGTTACCAGGTCTCGTGTTGCGAGAGCCAGTCTCTGTGCTG |
| | | CAGAGATTCAAGTAGACATGCAGCAACATCTTCTGTGTGAAG |
| | | CGGTACTCA |

### REFERENCES

1. Erlich, Y. & Zielinski, D. DNA Fountain enables a robust and efficient storage architecture. Science 355, 950-954 (2017).
2. Organick, L. et al. Random access in large-scale DNA data storage. Nat. Biotechnol. 36, 242-248 (2018).

1. Physically molecularly encrypted nucleic acid-encoded data comprising:
a) a plurality of double stranded polynucleotide fragments, comprising primer binding sites, said primer binding sites flanking nucleic acid encoded data
b) wherein at least a fraction of the double stranded polynucleotide fragments comprises one or more locker target sequences, optionally wherein the locker target sequence(s) and/or the primer binding site(s) of the polynucleotide sequence is/are part of the nucleic acid-encoded dataset
c) a locker, which is an oligonucleotide, having a modification that blocks amplification, optionally wherein the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide;
d) wherein the locker has sequence complementarity to the locker target in the double stranded polynucleotide fragments, and binds thereto, thereby preventing PCR-amplification of said double stranded polynucleotide fragments with primers from the primer binding sites;
e) wherein the plurality of double stranded polynucleotide fragments and the locker are mixed.

2. A method for physical molecular encryption of nucleic acid-encoded data, the method comprising the steps:
a) providing a digital dataset;
b) encoding the digital dataset to a nucleic acid code, thereby designing a nucleic acid-encoded dataset, wherein the nucleic acid-encoded dataset comprises a plurality of polynucleotide sequences of between 50-500 nucleotides, preferably 100-250 nucleotides, more preferably 150-200 nucleotides, wherein each of the polynucleotide sequences further comprise a forward and reverse primer binding site at the ends of the polynucleotide sequences that allows for subsequent PCR amplification wherein up to 100% of the individual polynucleotide sequences comprise at least one locker target sequence wherein the locker target sequence is located such that PCR amplification is blocked when a locker is bound to the locker target sequence, optionally wherein the locker target sequence and/or the primer binding site(s) of the polynucleotide sequence is/are part of the nucleic acid-encoded dataset, and/or optionally wherein the polynucleotide sequences comprise an indexing sequence;
c) synthesizing the designed plurality of polynucleotide sequences to provide for polynucleotide fragments, wherein the polynucleotide fragments are synthesized as single stranded polynucleotide fragments and subsequently made double stranded polynucleotide fragments or synthesized as double stranded polynucleotide fragments, preferably comprising an amplification step to provide for a desired concentration of double stranded polynucleotide fragments;
d) labeling the double stranded polynucleotide fragments with biotin to therewith provide for biotin-labeled double stranded polynucleotide fragments representing the nucleic acid-encoded dataset.

3. The method according to claim 2, further comprising the steps of:
e) providing a locker complex comprising a locker and a locker anchor, wherein the locker is an oligonucleotide, wherein the locker comprises a sequence complementary to the locker target sequence, wherein the locker anchor is an oligonucleotide comprising a biotin label, wherein the locker and locker anchor have sequences complementary with each other, wherein the locker of the locker complex comprises a modification that blocks amplification, optionally wherein the modification of the locker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide, preferably wherein the locker and locker anchor complementarity is between 1-30, preferably 5-25, more preferably 10-20, most preferably 12-16 nucleotides in length;
to therewith provide for biotin-labeled double stranded polynucleotide fragments representing the nucleic acid-encoded dataset and locker complex, which are preferably mixed, wherein PCR amplification of the biotin-labeled double stranded polynucleotide fragments is repressed in the biotin-labeled double stranded polynucleotide fragments comprising the locker target sequence by preferential binding of the locker.

4. The method according to claim 3, wherein the method further comprises the steps of:
f) providing a biotin-binding agent, preferably a biotin-binding protein, comprising one or more biotin binding domains, wherein a plurality of the biotin-binding protein is provided localized in proteinosome compartments;
g) localizing the biotin-labeled double stranded polynucleotide fragments and the locker complex in the proteinosome compartments, optionally wherein 50-100%, preferably 75-100%, more preferably 90-100% of the biotin-labeled double stranded polynucleotide fragments representing the nucleic acid encoded dataset are localized in a single proteinosome compartment and wherein the biotin-labeled double stranded polynucleotide fragments are distributed over the proteinosome compartments thereby covering the nucleic acid-encoded dataset at least once;
h) storing the proteinosome compartments containing the localized biotin-labeled double stranded polynucleotide fragments, locker complex and biotin-binding protein, to therewith provide for physically molecularly encrypted nucleic acid-encoded data.

5. A method comprising unlocking of physical molecular encrypted nucleic acid-encoded data, comprising the steps of:
i) providing proteinosome compartments as obtained in claim 4;
j) providing an unlocker, wherein the unlocker comprises an oligonucleotide sequence complementary to the locker of the locker complex, wherein the complementarity between the unlocker and locker is longer than the complementarity between the locker anchor and locker;
k) mixing the unlocker and the proteinosome compartments in a suspension allowing the unlocker to interact with the locker of the locker complex thereby forming a double stranded complex of the locker and the unlocker, resulting in substantially displacing the double stranded complex of the locker and the unlocker out of the proteinosome compartment by toehold mediated strand displacement, preferably washing the mixed suspension, thereby removing, by diffusion, the double stranded complex of the locker and the unlocker from the proteinosome compartments, preferably wherein the interaction between the locker and the unlocker is performed between 0-32 degrees Centigrade, preferably between 5-30 degrees Centigrade, more preferably between 15-25 degrees Centigrade,
therewith obtaining proteinosome compartments comprising biotin-labeled double stranded polynucleotide fragments representing the nucleic acid-encoded dataset and the locker anchor, from which the locker has been substantially removed;
l) amplifying the biotin-labeled double stranded polynucleotide fragments to therewith obtain amplified unlocked polynucleotide fragments, preferably using a forward universal primer and a reverse universal primer targeting the forward and reverse primer binding sites.
m) optionally wherein the amplified unlocked polynucleotide fragments and the proteinosome compartments comprising biotin-labeled double stranded polynucleotide fragments representing the nucleic acid-encoded dataset and the locker anchor are separated from each other, and wherein to the proteinosome compartments comprising biotin-labeled double stranded polynucleotide fragments and the locker anchor, a newly-provided locker is localized to therewith reconstitute the locker complex localized in the proteinosome compartments, and subsequently storing the reconstituted proteinosome compartments with biotin-labeled double stranded polynucleotide fragments and locker complex.

6. The method according to claim 5, wherein the unlocker comprises a modification that blocks amplification, optionally wherein the modification of the unlocker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide.

7. The method according to claim 2, wherein a plurality of digital datasets is provided wherein each of the digital datasets is encoded to individual nucleic acid codes, thereby designing a plurality of individual nucleic acid-encoded datasets.

8. The method according to claim 2, wherein a plurality of digital datasets is provided wherein each of the digital datasets is encoded to individual nucleic acid codes, thereby designing a plurality of individual nucleic acid-encoded datasets, wherein the individual nucleic acid-encoded datasets each comprise a plurality of polynucleotide sequences comprising unique indexing sequences for decoding, ordering of polynucleotide sequences and/or identifying the nucleic acid-encoded dataset to which the polynucleotide sequences belong.

9. The method according to claim 2, wherein the nucleic acid-encoded dataset is a DNA encoded dataset, and the biotin-labeled double stranded polynucleotide fragments are double stranded DNA fragments.

10. The method according to claim 2, wherein the locker target sequence is overlapping with one of the primer binding sites.

11. The method according to claim 2, wherein the polynucleotide sequences representing the nucleic acid-encoded dataset comprise more than one unique locker target sequence.

12.-17. (canceled)

18. The method according to claim 5, wherein the amplified unlocked polynucleotide fragments are sequenced to therewith obtain the polynucleotide sequences of the plurality of biotin-labeled double stranded polynucleotide fragments representing the nucleic acid-encoded dataset.

19. The method according to claim 18, wherein the obtained polynucleotide sequences of the amplified unlocked polynucleotide fragments comprising the nucleic acid-encoded dataset are decoded to restore the digital dataset as provided in step a), which decoding and restoring comprises ordering, optionally by means of the indexing sequence.

20. A locker complex comprising a locker and a locker anchor in accordance with claim 3.

21. Use of an unlocker, for unlocking physically encrypted nucleic acid-encoded data according to claim 1, wherein the unlocker is provided with a tag and comprises an oligonucleotide sequence complementary with the locker, and wherein the unlocker is allowed to interact with the locker thereby forming a double stranded complex of the locker and the unlocker, which double stranded complex is subsequently separated from the plurality of double stranded polynucleotide fragments by means of the tag.

22. Use of the unlocker in accordance with claim 21, wherein the tag is a biotin-label, and magnetic beads with a biotin-binding agent are provided for separating the double stranded complex of the locker and the unlocker from the plurality of double stranded polynucleotide fragments.

23. Use of the unlocker in accordance with claim 21, wherein the unlocker comprises a modification that blocks amplification, optionally wherein the modification of the unlocker that blocks amplification is selected from a 3′ inverted dT nucleotide a 3′ dideoxycytidine (ddC) a C3 phosphoramidite spacer, a 3′ amino modifier and a 3′ phosphorylation, preferably a 3′ inverted dT nucleotide.

24. A physically molecularly encrypted nucleic acid-encoded data system, comprising a plurality of double stranded polynucleotide fragments, and a locker as defined claim 1, wherein the mixture of double stranded polynucleotide fragments and the locker are combined in one container, and the unlocker is provided in a separate container

25. Physically molecular encrypted nucleic acid-encoded data in accordance with claim 1, wherein the locker is comprised in a locker complex, wherein the plurality of double stranded polynucleotide fragments are biotin-labeled, and wherein the plurality of double stranded polynucleotide fragments and locker complex is localized and compartmentalized in proteinosomes comprising biotin-binding agent, preferably a heat-resistant biotin binding protein.

26.-27. (canceled)

28. A physically molecularly encrypted nucleic acid-encoded data system, comprising a plurality of double stranded polynucleotide fragments, and a locker complex, and an unlocker, as defined in claim 2, wherein the double stranded polynucleotide fragments and the locker complex are combined in one container, and the unlocker is provided as a molecule in a separate container.
